(19) United States
(12) Patent Application Publication
(10) Pub. No.: US 2024/0425601 A1
(43) Pub. Date: Dec. 26, 2024

(54) COMPOSITIONS AND METHODS FOR TREATING AND PREVENTING DISEASE ASSOCIATED WITH AVB8 INTEGRIN

(71) Applicant: THE REGENTS OF THE UNIVERSITY OF CALIFORNIA, Oakland, CA (US)

(72) Inventors: Dean Sheppard, San Francisco, CA (US); Scott Bidlingmaier, San Francisco, CA (US); Bin Liu, San Francisco, CA (US); Amha Atakilit, San Francisco, CA (US)

(73) Assignee: THE REGENTS OF THE UNIVERSITY OF CALIFORNIA, Oakland, CA (US)

(21) Appl. No.: 18/274,069

(22) PCT Filed: Jan. 25, 2022
(86) PCT No.: PCT/US2022/013735

§ 371 Date: Jul. 25, 2023

Related U.S. Application Data
(60) Provisional application No. 63/141,703, filed on Jan. 26, 2021.

(51) Int. Cl. C07K 16/28; A61K 39/395; A61K 45/06; A61P 35/00

(52) U.S. Cl. CPC C07K 16/2839; A61K 39/3955; A61K 45/06; A61P 35/00; C07K 2317/24

(57) ABSTRACT

Compositions and methods comprising integrin β8 antibodies are provided.

### CROSS-REFERENCES TO RELATED APPLICATION

This application is a U.S. 371 National Phase of International Application No. PCT/US2022/013735 filed Jan. 25, 2022, which claims priority to U.S. Provisional Application No. 63/141,703, filed Jan. 26, 2021, the disclosures of which are hereby incorporated by reference in their entirety for all purposes.

### SEQUENCE LISTING

The instant application contains a Sequence Listing which has been submitted electronically in ASCII format and is hereby incorporated by reference in its entirety. Said ASCII copy, created on Jan. 24, 2022, is named 081906-1287440-241202WO_SL.txt and is 98,745 bytes in size.

### BACKGROUND

Members of the integrin family recognize a variety of spatially-restricted extracellular ligands. Classically, ligation of integrins activates cytoplasmic signals in the integrin-expressing cell and contributes to cell adhesion, migration, proliferation and survival. At least two members of this family, αvβ6 and αvβ8, perform an additional function, activation of latent complexes of transforming growth factor β. In effect, this process allows integrins on one cell to activate signals on adjacent (in the case of αvβ6) or nearby cells (in the case of αvβ8). Integrin-mediated TGFβ activation has been shown to play roles in, for example, modulating tissue fibrosis, acute lung injury and pulmonary emphysema.

### BRIEF SUMMARY

In one aspect, the disclosure features an isolated antibody that specifically binds to human integrin β8 and inhibits adhesion of latency associated peptide (LAP) to αvβ8, wherein the isolated antibody comprises: (1) a heavy chain complementary determining region 1 (HCDR1) having a sequence of any one of SEQ ID NOS:1, 5, and 6; (2) an HCDR2 having a sequence of any one of SEQ ID NOS:2, 4, and 7; (3) an HCDR3 having the sequence of SEQ ID NO:3; (4) a light chain complementary determining region 1 (LCDR1) having a sequence of any one of SEQ ID NOS:8, 11, 13, and 14; (5) a LCDR2 having a sequence of any one of SEQ ID NOS:9 and 12; and (6) a LCDR3 having the sequence of SEQ ID NO:10.

In some embodiments, the antibody comprises an HCDR1 having the sequence of SEQ ID NO:1, an HCDR2 having the sequence of SEQ ID NO: 2, and an HCDR3 having the sequence of SEQ ID NO: 3.

In some embodiments, the antibody comprises an HCDR1 having the sequence of SEQ ID NO:1, an HCDR2 having the sequence of SEQ ID NO: 4, and an HCDR3 having the sequence of SEQ ID NO: 3.

In some embodiments, the antibody comprises an HCDR1 having the sequence of SEQ ID NO: 5, an HCDR2 having the sequence of SEQ ID NO: 2, and an HCDR3 having the sequence of SEQ ID NO: 3.

In some embodiments, the antibody comprises an HCDR1 having the sequence of SEQ ID NO: 6, an HCDR2 having the sequence of SEQ ID NO: 2, and an HCDR3 having the sequence of SEQ ID NO: 3.

In some embodiments, the antibody comprises an HCDR1 having the sequence of SEQ ID NO:1, an HCDR2 having the sequence of SEQ ID NO: 7, and an HCDR3 having the sequence of SEQ ID NO: 3.

In some embodiments, the antibody comprises a LCDR1 having the sequence of SEQ ID NO: 8, a LCDR2 having the sequence of SEQ ID NO: 9, and a LCDR3 having the sequence of SEQ ID NO: 10.

In some embodiments, the antibody comprises a LCDR1 having the sequence of SEQ ID NO: 11, a LCDR2 having the sequence of SEQ ID NO: 9, and a LCDR3 having the sequence of SEQ ID NO: 10.

In some embodiments, the antibody comprises a LCDR1 having the sequence of SEQ ID NO: 8, a LCDR2 having the sequence of SEQ ID NO: 12, and a LCDR3 having the sequence of SEQ ID NO: 10.

In some embodiments, the antibody comprises a LCDR1 having the sequence of SEQ ID NO: 13, a LCDR2 having the sequence of SEQ ID NO: 12, and a LCDR3 having the sequence of SEQ ID NO: 10.

In some embodiments, the antibody comprises a LCDR1 having the sequence of SEQ ID NO: 14, a LCDR2 having the sequence of SEQ ID NO: 9, and a LCDR3 having the sequence of SEQ ID NO: 10.

In some embodiments, the antibody comprises a heavy chain variable region having at least 90% identity to a sequence of any one of SEQ ID NOS:15-19. In some embodiments, the antibody comprises a light chain variable region having at least 90% identity to a sequence of any one of SEQ ID NOS:20-25.

Further, the antibody can comprise an Fc polypeptide having at least 90% identity to a sequence of any one of SEQ ID NOS:47-50. The Fc polypeptide can comprise amino acid substitutions L234A and L235A. Further, the Fc polypeptide can comprise the amino acid substitution N297A.

In some embodiments, the antibody comprises: (1) an HCDR1 having the sequence of SEQ ID NO: 1; (2) an HCDR2 having the sequence of SEQ ID NO: 4; (3) an HCDR3 having the sequence of SEQ ID NO: 3; (4) a LCDR1 having the sequence of SEQ ID NO: 8; (5) a LCDR2 having the sequence of SEQ ID NO: 12; and (6) a LCDR3 having the sequence of SEQ ID NO: 10. The antibody can comprise a heavy chain variable region having at least 90% identity to the sequence of SEQ ID NO: 16. The antibody can comprise a light chain variable region having at least 90% identity to the sequence of SEQ ID NO: 22. In particular embodiments, the antibody can comprise an Fc polypeptide having at least 90% identity to a sequence of any one of SEQ ID NOS:47-50. The Fc polypeptide can comprise amino acid substitutions L234A and L235A. Moreover, the Fc polypeptide can comprise the amino acid substitution N297A.

In some embodiments of this aspect, the antibody is a monoclonal antibody. In some embodiments, the antibody is a humanized antibody. In some embodiments, the antibody cross-reacts with mouse integrin β8. In some embodiments, the antibody blocks TGFβ activation. In some embodiments, the antibody antagonizes binding of LAP to αvβ8 with an IC₅₀ below 5 nM. In some embodiments, the antibody comprises one or more human framework regions.

In another aspect, the disclosure features an isolated nucleic acid encoding the isolated antibody described herein. In another aspect, the disclosure features an expression vector comprising the nucleic acid that encodes the isolated antibody described herein. In yet another aspect, the disclosure features an isolated host cell comprising the expression vector.

In another aspect, the disclosure features a pharmaceutical composition comprising the isolated antibody described herein and a pharmaceutically acceptable carrier.

In another aspect, the disclosure features a method of reducing TGFβ activation in a human in need thereof, the method comprising administering a therapeutically effective amount of the isolated antibody described herein or the pharmaceutical composition comprising the isolated antibody to the human, thereby reducing TGFβ activation in the human. In some embodiments, the human has a disease selected from the group consisting of asthma, multiple sclerosis or acute lung injury and at least one symptom of the disease is ameliorated by the reduced TGFβ activation. In some embodiments, the human has a disease selected from the group consisting of rheumatoid arthritis, psoriasis and chronic obstructive pulmonary disease and at least one symptom of the disease is ameliorated by the reduced TGFβ activation.

In another aspect, the disclosure features a method of treating a cancer in a human, the method comprising administering to the human a therapeutically effective amount of the isolated antibody described herein or the pharmaceutical composition comprising the isolated antibody to the human, thereby treating the cancer. In some embodiments, the cancer is a metastatic cancer. In some embodiments, the cancer is a solid tumor cancer. In some embodiments, the method enhances an immune response to the cancer in the human. In certain embodiments of the method, the isolated antibody is administered in combination with an immunomodulator (e.g., a PD1 antagonist, a PDL1 antagonist, a CTLA4 antagonist, a 41BB agonist). In certain embodiments of the method, the isolated antibody is administered in combination with radiotherapy. In certain embodiments of the method, the isolated antibody is administered in combination with chemotherapy.

### BRIEF DESCRIPTION OF THE DRAWINGS

FIG. 1 shows that ADWA16 was more potent at inhibiting adhesion of L229 cells expressing αvβ8 to TGFb1 LAP, than was ADWA11.

FIGS. 2A and 2B show that unlabeled ADWA11 effectively competed for binding of labelled ADWA11 to L229 cells expressing αvβ8, but unlabeled ADWA16 did not, demonstrating that the two antibodies recognize different epitopes.

FIGS. 3A-3E show association/dissociation curves for each antibody.

FIG. 4 shows that humanized, affinity-matured IgGs (ADWA16-1, ADWA16-2, ADWA16-3, and ADWA16-4) showed affinity for cell surface-expressed human αvβ8 that was equivalent to the parent murine IgG (ADWA16-chimera and ADWA16).

FIGS. 5A and 5B show that ADWA16-3 and ADWA16-3.2 displayed affinity for recombinant human αvβ8 that is equivalent to the parent murine IgG (ADWA16-chimera) and significantly improved affinity for recombinant mouse αvβ8.

FIG. 6 shows that ADWA16-3 and ADWA16-3.2 displayed affinity for cell surface-expressed human αvβ8 that was equivalent to the parent murine IgG (ADWA16-chimera and ADWA16).

FIG. 7 shows that ADWA16-3.2 IgG displayed significantly improved binding to surface-expressed mouse αvβ8 compared to the parent murine IgG (ADWA16).

FIG. 8 shows that ADWA16-3 and ADWA16-3.2 did not bind to αvβ3, αvβ5, or αvβ6-expressing SW480 cells, but only displayed binding to SNB19 cells expressing αvβ3, αvβ5, and αvβ8.

FIGS. 9A-9C show that ADWA16-3 and ADWA16-3.2 retained the high potency of the murine parent ADWA16 to inhibit activation of TGFβ.

FIGS. 10A and 10B show that ADWA16-3.2 retained low nanomolar potency in inhibiting adhesion of SNB19 cells to TGFβ1-LAP.

FIG. 11 shows that ADWA16-3.2 displayed improved thermal stability compared to the parent murine antibody ADWA-16.

FIG. 12 shows the binding of ADWA16-3.2 and ADWA11 in SNB19 human astrocytoma cells.

FIGS. 13A and 13B show ADWA16-3.2 and ADWA11 inhibited adhesion of SNB19 cells to TGFb1 LAP.

### DETAILED DESCRIPTION

### I. Introduction

The inventors have discovered new antibodies that bind to both murine and human integrin β8, and that are more potent inhibitors of αvβ8 ligand adhesion. The antibodies described herein can be used to treat or prevent diseases associated with αvβ8 expression, such as cancer and pulmonary fibrosis.

### I. Definitions

An “antagonist” refers to an agent that binds to an integrin (e.g., αvβ8) and partially or totally blocks stimulation, decreases, prevents, delays activation, inactivates, desensitizes, or down regulates the activity of the integrin.

The terms “identical” or percent “identity,” in the context of two or more nucleic acids or polypeptide sequences, refer to two or more sequences or subsequences that are the same or have a specified percentage of amino acid residues or nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST 2.0 sequence comparison algorithms with default parameters described below, or by manual alignment and visual inspection (see, e.g., NCBI web site ncbi.nlm.nih.gov/BLAST/or the like). Such sequences are then said to be “substantially identical.” As described below, the preferred algorithms can account for gaps and the like. Preferably, identity exists over a region that is at least about 25 amino acids or nucleotides in length, or more preferably over a region that is 50-100 or more amino acids or nucleotides in length.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Preferably, default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well-known in the art.

An algorithm that is suitable for determining percent sequence identity and sequence similarity are the BLAST and BLAST 2.0 algorithms, which are described in Altschul et al., Nuc. Acids Res. 25:3389-3402 (1977) and Altschul et al., J. Mol. Biol. 215:403-410 (1990), respectively. BLAST and BLAST 2.0 are used, with the parameters described herein, to determine percent sequence identity for the nucleic acids and proteins of the disclosure. Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=−4 and a comparison of both strands. For amino acid sequences, the BLASTP program uses as defaults a wordlength of 3, and expectation (E) of 10, and the BLOSUM62 scoring matrix (see Henikoff & Henikoff, Proc. Natl. Acad. Sci. USA 89:10915 (1989)) alignments (B) of 50, expectation (E) of 10, M=5, N=−4, and a comparison of both strands.

The term “nucleic acid” refers to deoxyribonucleotides or ribonucleotides and polymers thereof in either single- or double-stranded form, and complements thereof. The term encompasses nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, 2-O-methyl ribonucleotides, peptide-nucleic acids (PNAs).

Unless otherwise indicated, a particular nucleic acid sequence also implicitly encompasses conservatively modified variants thereof (e.g., degenerate codon substitutions) and complementary sequences, as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J Biol. Chem. 260:2605-2608 (1985); Rossolini et al., Mol. Cell. Probes 8:91-98 (1994)).

The terms “polypeptide,” “peptide” and “protein” are used interchangeably herein to refer to a polymer of amino acid residues. The terms encompass to amino acid polymers in which one or more amino acid residue is an artificial chemical mimetic of a corresponding naturally occurring amino acid, as well as to naturally occurring amino acid polymers and non-naturally occurring amino acid polymer.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

The term “recombinant” when used with reference, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Thus, for example, recombinant cells express genes that are not found within the native (non-recombinant) form of the cell or express native genes that are otherwise abnormally expressed, under expressed or not expressed at all.

An antibody as described herein can consist of one or more polypeptides substantially encoded by immunoglobulin genes or fragments of immunoglobulin genes. The recognized immunoglobulin genes include the kappa, lambda, alpha, gamma, delta, epsilon and mu constant region genes, as well as myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively. In some embodiments, the antibody is IgG (e.g., IgG1, IgG2, IgG3, IgG4), IgM, IgA, IgD, or IgE.

A typical immunoglobulin (antibody) structural unit is known to comprise a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (V_{L}) and variable heavy chain (V_{H}) refer to these light and heavy chains respectively.

The term “antibody” as used herein includes antibody fragments that retain binding specificity. For example, there are a number of well characterized antibody fragments. Thus, for example, pepsin digests an antibody C-terminal to the disulfide linkages in the hinge region to produce F(ab)′2, a dimer of Fab which itself is a light chain joined to V_{H}-CH1 by a disulfide bond. The F(ab)′2 may be reduced under mild conditions to break the disulfide linkage in the hinge region thereby converting the (Fab′)₂ dimer into an Fab′ monomer. The Fab′ monomer is essentially an Fab with part of the hinge region (see, Fundamental Immunology, W. E. Paul, ed., Raven Press, N.Y. (1993), for a more detailed description of other antibody fragments). While various antibody fragments are defined in terms of the digestion of an intact antibody, one of skill will appreciate that fragments can be synthesized de novo either chemically or by utilizing recombinant DNA methodology. Thus, the term antibody, as used herein also includes antibody fragments either produced by the modification of whole antibodies or synthesized using recombinant DNA methodologies.

In an antibody, substitution variants have at least one amino acid residue removed and a different residue inserted in its place. The sites of greatest interest for substitutional mutagenesis include the hypervariable regions, but framework alterations are also contemplated. Examples of conservative substitutions are described above.

Substantial modifications in the biological properties of the antibody are accomplished by selecting substitutions that differ significantly in their effect on maintaining (a) the structure of the polypeptide backbone in the area of the substitution, for example, as a β-sheet or helical conformation, (b) the charge or hydrophobicity of the molecule at the target site, or (c) the bulk of the side chain. Naturally occurring residues are divided into groups based on common side-chain properties:
(1) Non-polar: Norleucine, Met, Ala, Val, Leu, Ile;
(2) Polar without charge: Cys, Ser, Thr, Asn, Gln;
(3) Acidic (negatively charged): Asp, Glu;
(4) Basic (positively charged): Lys, Arg;
(5) Residues that influence chain orientation: Gly, Pro; and
(6) Aromatic: Trp, Tyr, Phe, His.
   Non-conservative substitutions are made by exchanging a member of one of these classes for another class.

One type of substitution that can be made is to change one or more cysteines in the antibody, which may be chemically reactive, to another residue, such as, without limitation, alanine or serine. For example, there can be a substitution of a non-canonical cysteine. The substitution can be made in a CDR or framework region of a variable domain or in the constant region of an antibody. In some embodiments, the cysteine is canonical (e.g., involved in disulfide bond formation). Any cysteine residue not involved in maintaining the proper conformation of the antibody also may be substituted, generally with serine, to improve the oxidative stability of the molecule and prevent aberrant cross-linking. Conversely, cysteine bond(s) may be added to the antibody to improve its stability, particularly where the antibody is an antibody fragment such as an Fv fragment.

Antibodies include V_{H}-V_{L} dimers, including single chain antibodies (antibodies that exist as a single polypeptide chain), such as single chain Fv antibodies (sFv or scFv) in which a variable heavy and a variable light region are joined together (directly or through a peptide linker) to form a continuous polypeptide. The single chain Fv antibody is a covalently linked V_{H}-V_{L} which may be expressed from a nucleic acid including V_{H}- and V_{L}-encoding sequences either joined directly or joined by a peptide-encoding linker (e.g., Huston, et al. Proc. Nat. Acad. Sci. USA, 85:5879-5883, 1988). While the V_{H} and V_{L} are connected to each as a single polypeptide chain, the V_{H} and V_{L} domains associate non-covalently. Alternatively, the antibody can be another fragment. Other fragments can also be generated, e.g., using recombinant techniques, as soluble proteins or as fragments obtained from display methods. Antibodies can also include diantibodies and miniantibodies. Antibodies of the disclosure also include heavy chain dimers, such as antibodies from camelids. In some embodiments an antibody is dimeric. In other embodiments, the antibody may be in a monomeric form that has an active isotype. In some embodiments the antibody is in a multivalent form, e.g., a trivalent or tetravalent form.

As used herein, the terms “variable region” and “variable domain” refer to the portions of the light and heavy chains of an antibody that include amino acid sequences of complementary determining regions (CDRs, e.g., HCDR1, HCDR2, HCR3, LCDR1, LCDR2, and LCDR3) and framework regions (FRs). The variable region for the heavy and light chains is commonly designated V_{H} and V_{L}, respectively. The variable region is included on Fab, F(ab′)₂, Fv and scFv antibody fragments described herein, and involved in specific antigen recognition.

As used herein, “complementarity-determining region (CDR)” refers to the three hypervariable regions in each chain that interrupt the four framework regions established by the light and heavy chain variable regions. The CDRs are primarily responsible for binding to an epitope of an antigen. The CDRs of each chain are typically referred to as CDR1, CDR2, and CDR3, numbered sequentially starting from the N-terminus, and are also typically identified by the chain in which the particular CDR is located. Thus, a V_{H} CDR3 is located in the variable domain of the heavy chain of the antibody in which it is found, whereas a V_{L} CDR1 is the CDR1 from the variable domain of the light chain of the antibody in which it is found.

The sequences of the framework regions of different light or heavy chains are relatively conserved within a species. The framework region of an antibody, that is the combined framework regions of the constituent light and heavy chains, serves to position and align the CDRs in three dimensional space.

The amino acid sequences of the CDRs and framework regions can be determined using various well known definitions in the art, e.g., Kabat, North method (see, e.g., North et al., J Mol Biol. 406(2):228-256, 2011), Chothia, international ImMunoGeneTics database (IMGT), and AbM (see, e.g., Johnson et al., supra; Chothia & Lesk, 1987, Canonical structures for the hypervariable regions of immunoglobulins. J Mol. Biol. 196, 901-917; Chothia C. et al., 1989, Conformations of immunoglobulin hypervariable regions. Nature 342, 877-883; Chothia C. et al., 1992, structural repertoire of the human V_{H} segments J. Mol. Biol. 227, 799-817; Al-Lazikani et al., J. Mol. Biol 1997, 273(4)). Definitions of antigen combining sites are also described in the following: Ruiz et al., IMGT, the international ImMunoGeneTics database. Nucleic Acids Res., 28, 219-221 (2000); and Lefranc, M.-P. IMGT, the international ImMunoGeneTics database. Nucleic Acids Res. Jan 1;29(1):207-9 (2001); MacCallum et al, Antibody-antigen interactions: Contact analysis and binding site topography, J. Mol. Biol., 262 (5), 732-745 (1996); and Martin et al, Proc. Natl Acad. Sci. USA, 86, 9268-9272 (1989); Martin, et al, Methods Enzymol., 203, 121-153, (1991); Pedersen et al, Immunomethods, 1, 126, (1992); and Rees et al, In Stemnberg M. J. E. (ed.), Protein Structure Prediction. Oxford University Press, Oxford, 141-172 1996).

As used herein, “chimeric antibody” refers to an immunoglobulin molecule in which (a) the constant region, or a portion thereof, is altered, replaced or exchanged so that the antigen binding site (variable region) is linked to a constant region of a different or altered class, effector function and/or species, or an entirely different molecule which confers new properties to the chimeric antibody, e.g., an enzyme, toxin, hormone, growth factor, drug, etc.; or (b) the variable region, or a portion thereof, is altered, replaced or exchanged with a variable region, or portion thereof, having a different or altered antigen specificity; or with corresponding sequences from another species or from another antibody class or subclass.

As used herein, “humanized antibody” refers to an immunoglobulin molecule in CDRs from a donor antibody are grafted onto human framework sequences. Humanized antibodies may also comprise residues of donor origin in the framework sequences. The humanized antibody can also comprise at least a portion of a human immunoglobulin constant region. Humanized antibodies may also comprise residues which are found neither in the recipient antibody nor in the imported CDR or framework sequences. Humanization can be performed using methods known in the art (e.g., Jones et al., Nature 321:522-525; 1986; Riechmann et al., Nature 332:323-327, 1988; Verhoeyen et al., Science 239:1534-1536, 1988); Presta, Curr. Op. Struct. Biol. 2:593-596, 1992; U.S. Pat. No. 4,816,567), including techniques such as “superhumanizing” antibodies (Tan et al., J. Immunol. 169: 1119, 2002) and “resurfacing” (e.g., Staelens et al., Mol. Immunol. 43: 1243, 2006; and Roguska et al., Proc. Natl. Acad. Sci USA 91: 969, 1994).

The terms “antigen,” “immunogen,” “antibody target,” “target analyte,” and like terms are used herein to refer to a molecule, compound, or complex that is recognized by an antibody, i.e., can be specifically bound by the antibody. The term can refer to any molecule that can be specifically recognized by an antibody, e.g., a polypeptide, polynucleotide, carbohydrate, lipid, chemical moiety, or combinations thereof (e.g., phosphorylated or glycosylated polypeptides, etc.). One of skill will understand that the term does not indicate that the molecule is immunogenic in every context, but simply indicates that it can be targeted by an antibody.

Antibodies bind to an “epitope” on an antigen. The epitope is the localized site on the antigen that is recognized and bound by the antibody. Epitopes can include a few amino acids or portions of a few amino acids, e.g., 5 or 6, or more, e.g., 20 or more amino acids, or portions of those amino acids. In some cases, the epitope includes non-protein components, e.g., from a carbohydrate, nucleic acid, or lipid. In some cases, the epitope is a three-dimensional moiety. Thus, for example, where the target is a protein, the epitope can be comprised of consecutive amino acids, or amino acids from different parts of the protein that are brought into proximity by protein folding (e.g., a discontinuous epitope). The same is true for other types of target molecules that form three-dimensional structures. An epitope typically includes at least 3, and more usually, at least 5 or 8-10 amino acids in a unique spatial conformation. Methods of determining spatial conformation of epitopes include, for example, x-ray crystallography and 2-dimensional nuclear magnetic resonance. See, e.g., Epitope Mapping Protocols in Methods in Molecular Biology, Vol. 66, Glenn E. Morris, Ed (1996).

A “label” or a “detectable moiety” is a diagnostic agent or component detectable by spectroscopic, radiological, photochemical, biochemical, immunochemical, chemical, or other physical means. Exemplary labels include radiolabels (e.g., ¹¹¹In, ^{99m}Tc, ¹³¹I, ⁶⁷Ga) and other FDA-approved imaging agents. Additional labels include ³²P, fluorescent dyes, electron-dense reagents, enzymes, biotin, digoxigenin, or haptens and proteins or other entities which can be made detectable, e.g., by incorporating a radiolabel into the targeting agent. Any method known in the art for conjugating a nucleic acid or nanocarrier to the label may be employed, e.g., using methods described in Hermanson, Bioconjugate Techniques 1996, Academic Press, Inc., San Diego.

A “labeled” or “tagged” antibody or agent is one that is bound, either covalently, through a linker or a chemical bond, or noncovalently, through ionic, van der Waals, electrostatic, or hydrogen bonds to a label such that the presence of the antibody or agent may be detected by detecting the presence of the label bound to the antibody or agent.

Techniques for conjugating detectable and therapeutic agents to antibodies are well known (see, e.g., Amon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. 1985); Hellstrom et al., “Antibodies For Drug Delivery” in Controlled Drug Delivery (2nd Ed.), Robinson et al. (eds.), pp. 623-53 (Marcel Dekker, Inc. 1987); Thorpe, “Antibody Carriers Of Cytotoxic Agents In Cancer Therapy: A Review” in Monoclonal Antibodies '84: Biological And Clinical Applications, Pinchera et al. (eds.), pp. 475-506 (1985); and Thorpe et al., “The Preparation And Cytotoxic Properties Of Antibody-Toxin Conjugates”, Immunol. Rev., 62:119-58 (1982)).

The terms “specific for,” “specifically binds,” and like terms refer to a molecule (e.g., antibody or antibody fragment) that binds to a target with at least 2-fold greater affinity than non-target compounds, e.g., at least any of 4-fold, 5-fold, 6-fold, 7-fold, 8-fold, 9-fold, 10-fold, 20-fold, 25-fold, 50-fold, or 100-fold greater affinity. For example, an antibody that specifically binds a target (e.g., human or murine αvβ8) will typically bind the target with at least a 2-fold greater affinity than a non-target. Specificity can be determined using standard methods, e.g., solid-phase ELISA immunoassays (see, e.g., Harlow & Lane, Using Antibodies, A Laboratory Manual (1998) for a description of immunoassay formats and conditions that can be used to determine specific immunoreactivity).

The term “binds” with respect to an antibody target (e.g., antigen, analyte, immune complex), typically indicates that an antibody binds a majority of the antibody targets in a pure population (assuming appropriate molar ratios). For example, an antibody that binds a given antibody target typically binds to at least ⅔ of the antibody targets in a solution (e.g., at least any of 75, 80, 85, 90, 91, 92, 93, 94, 95, 96, 97, 98, 99, or 100%). One of skill will recognize that some variability will arise depending on the method and/or threshold of determining binding.

A “control” sample or value refers to a sample that serves as a reference, usually a known reference, for comparison to a test sample. For example, a test sample can be taken from a test condition, e.g., in the presence of a test compound, and compared to samples from known conditions, e.g., in the absence of the test compound (negative control), or in the presence of a known compound (positive control). A control can also represent an average value or a range gathered from a number of tests or results. One of skill in the art will recognize that controls can be designed for assessment of any number of parameters. For example, a control can be devised to compare therapeutic benefit based on pharmacological data (e.g., half-life) or therapeutic measures (e.g., comparison of benefit and/or side effects). Controls can be designed for in vitro applications. One of skill in the art will understand which controls are valuable in a given situation and be able to analyze data based on comparisons to control values. Controls are also valuable for determining the significance of data. For example, if values for a given parameter are widely variant in controls, variation in test samples will not be considered as significant.

The terms “therapeutically effective dose,” “effective dose,” or “therapeutically effective amount” herein is meant a dose that produces effects for which it is administered.

The exact dose and formulation will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Remington: The Science and Practice of Pharmacy, 20th Edition, Gennaro, Editor (2003), and Pickar, Dosage Calculations (1999)). For example, for the given parameter, a therapeutically effective amount will show an increase or decrease of therapeutic effect at least any of 5%, 10%, 15%, 20%, 25%, 40%, 50%, 60%, 75%, 80%, 90%, or at least 100%. Therapeutic efficacy can also be expressed as “-fold” increase or decrease. For example, a therapeutically effective amount can have at least any of a 1.2-fold, 1.5-fold, 2-fold, 5-fold, or more effect over a control.

The term “pharmaceutically acceptable salts” or “pharmaceutically acceptable carrier” is meant to include salts of the active compounds which are prepared with relatively nontoxic acids or bases, depending on the particular substituents found on the antibodies described herein. Examples of pharmaceutically acceptable base addition salts include sodium, potassium, calcium, ammonium, organic amino, or magnesium salt, or a similar salt. When compounds of the present disclosure contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, e.g., Berge et al., Journal of Pharmaceutical Science 66:1-19 (1977)). Certain specific compounds of the present disclosure contain both basic and acidic functionalities that allow the compounds to be converted into either base or acid addition salts. Other pharmaceutically acceptable carriers known to those of skill in the art are suitable for the present disclosure.

The term “reduce,” “reducing,” or “reduction,” when used in the context of αvβ8-mediate TGFβ activation refers to any detectable negative change or decrease in quantity of a parameter that reflects TGFβ activation, compared to a standard value obtained under the same conditions but in the absence of an antibody as described herein (e.g., anti-αvβ8 antibodies). The level of this decrease following exposure to an antibody as described herein (e.g., anti-αvβ8 antagonists, anti-αvβ8 antibodies and immunoconjugates) is, in some embodiments, at least 10%, 20%, 30%, 40%, 50%, 60%, 70%, 80%, 90%, 95%, or 100%.

The term “compete”, as used herein with regard to an antibody, means that a first antibody, or an antigen-binding portion thereof, competes for binding with a second antibody, or an antigen-binding portion thereof, where binding of the first antibody with its cognate epitope is detectably decreased in the presence of the second antibody compared to the binding of the first antibody in the absence of the second antibody. The alternative, where the binding of the second antibody to its epitope is also detectably decreased in the presence of the first antibody, can, but need not be the case. That is, a first antibody can inhibit the binding of a second antibody to its epitope without that second antibody inhibiting the binding of the first antibody to its respective epitope. However, where each antibody detectably inhibits the binding of the other antibody with its cognate epitope or ligand, whether to the same, greater, or lesser extent, the antibodies are said to “cross-compete” with each other for binding of their respective epitope(s). Both competing and cross-competing antibodies are encompassed by the present disclosure. Regardless of the mechanism by which such competition or cross-competition occurs (e.g., steric hindrance, conformational change, or binding to a common epitope, or portion thereof, and the like), the skilled artisan would appreciate, based upon the teachings provided herein, that such competing and/or cross-competing antibodies are encompassed and can be useful for the methods disclosed herein.

Numerous types of competitive binding assays are known, for example: solid phase direct or indirect radioimmunoassay (RIA), solid phase direct or indirect enzyme immunoassay (EIA), sandwich competition assay (see Stahli et al., Methods in Enzymology 9:242-253 (1983)); solid phase direct biotin-avidin EIA (see Kirkland et al., J. Immunol. 137:3614-3619 (1986)); solid phase direct labeled assay, solid phase direct labeled sandwich assay (see Harlow and Lane, Antibodies, A Laboratory Manual, Cold Spring Harbor Press (1988)); solid phase direct label RIA using I-125 label (see Morel et al., Molec. Immunol. 25(1):7-15 (1988)); solid phase direct biotin-avidin EIA (Cheung et al., Virology 176:546-552 (1990)); and direct labeled RIA (Moldenhauer et al., Scand. J Immunol. 32:77-82 (1990)). Typically, such an assay involves the use of purified antigen bound to a solid surface or cells bearing either of these, an unlabelled test immunoglobulin and a labeled reference immunoglobulin. Competitive inhibition is measured by determining the amount of label bound to the solid surface or cells in the presence of the test immunoglobulin. Usually the test immunoglobulin is present in excess. Antibodies identified by competition assay (competing antibodies) include antibodies binding to the same epitope as the reference antibody and antibodies binding to an adjacent epitope sufficiently proximal to the epitope bound by the reference antibody for steric hindrance to occur. Usually, when a competing antibody is present in excess, it will inhibit specific binding of a reference antibody to a common antigen by at least 50 or 75%.

III. Antibodies that Bind Integrin β8

Antibodies (including antibody fragments) that specifically bind to human integrin β8 are provided, as well as methods for treating or preventing diseases for which decrease of TGFβ activation has an ameliorative effect. “Integrin β8” is used interchangeably with 38 and beta-8. The human integrin β8 protein sequence can be found at Uniprot accession number P26012, while the murine integrin β8 sequence has Uniprot accession number Q0VBD0. See, also, Moyle et al. Journal of Biological Chemistry 266:19650-19658 (1991); Nishimura et al., J Biological Chemistry 269:28708-28715 (1994).

In some embodiments, an antibody that specifically binds to integrin β8 and inhibits (partially or completely blocks) binding of latency associated peptide (LAP) to αvβ8 is provided. LAP is a ligand for αvβ8. See, e.g., Sheppard, Cancer and Metastasis Reviews 24(3):395-402 (2005); Lu et al. J Cell Sci 115:4641-4648 (2002). Antibodies can antagonize LAP binding to αvβ8 with an IC₅₀ of, for example, less than, e.g., 10, 5, 1, 0.1 nM or lower.

In some embodiments, an antibody of the disclosure specifically binds to mouse integrin β8 and/or human integrin β8. One advantage of such antibodies is that clinical data can be generated for these antibodies in mice as well as humans. In some embodiments, an antibody of the disclosure binds to human integrin β8.

One aspect of blockage of LAP binding to αvβ8 in a cell can be that the antibodies prevent or reduce TGFβ activation by the cell. Thus, in some embodiments, the antibodies described herein are useful for decreasing TGFβ activation in a cell or an animal (e.g., a mouse or human).

In some embodiments, antibodies of the disclosure can comprise sequences of a heavy chain complementary determining region 1 (HCDR1), an HCDR2, an HCDR3, a light chain complementary determining region 1 (LCDR1), a LCDR2, a LCDR3, a heavy chain variable region (V_{H}), and/or a light chain variable region (V_{L}) as described in Table 1. The CDRs described in Table 1 are determined by North method (see, e.g., North et al., J Mol Biol. 406(2):228-256, 2011).

TABLE 1

| Antibody | HCDR1 | HCDR2 | HCDR3 | VH | LCDR1 | LCDR2 | LCDR3 | VL |
|---|---|---|---|---|---|---|---|---|
| ADWA16- | IFSSYW | YINPTT | ATEGG | EVQLVESGGGLVQPGGSLRLSC | RASQDI | YYTSRL | QQFSEL | DIQMTQSPSSLSASVGDRVTITCR |
| 1 | AVSGY | GYTE | NWEDY | AVSGYIFSSYWVYWVRQAPGK | SNYIN | YS (SEQ | PRT | ASQDISNYINWYQQKPGKAPKLL |
| | VY | (SEQ ID | (SEQ ID | GLEWVGYINPTTGYTEYADSVK | (SEQ ID | ID NO: 9) | (SEQ ID | IYYTSRLYSGVPSRFSGSGSGTDF |
| | (SEQ ID | NO: 2) | NO: 3) | GRFTISRDNSKNTLYLQMNSLR | NO: 8) | | NO: 10) | TLTISSLQPEDFATYYCQQFSELP |
| | NO: 1) | | | AEDTAVYYCATEGGNWEDYW | | | | RTFGQGTKVEIK (SEQ ID NO: 20) |
| | | | | GQGTLVTVSS (SEQ ID NO: 15) | | | | |
| ADWA16- | IFSSYW | YINPTT | ATEGG | EVQLVESGGGLVQPGGSLRLSC | RASKDI | YYTSRL | QQFSEL | DIQMTQSPSSLSASVGDRVTITCR |
| 2 | AVSGY | GYTE | NWEDY | AVSGYIFSSYWVYWVRQAPGK | SNYLN | YS (SEQ | PRT | ASKDISNYLNWYQQKPGKAPKL |
| | VY | (SEQ ID | (SEQ ID | GLEWVGYINPTTGYTEYADSVK | (SEQ ID | ID NO: 9) | (SEQ ID | LIYYTSRLYSGVPSRFSGSGSGTD |
| | (SEQ ID | NO: 2) | NO: 3) | GRFTISRDNSKNTLYLQMNSLR | NO: 11) | | NO: 10) | FTLTISSLQPEDFATYYCQQFSEL |
| | NO: 1) | | | AEDTAVYYCATEGGNWEDYW | | | | PRTFGQGTKVEIK (SEQ ID |
| | | | | GQGTLVTVSS (SEQ ID NO: 15) | | | | NO: 21) |
| ADWA16- | IFSSYW | YINPTT | ATEGG | EVQLVESGGGLVQPGGSLRLSC | RASQDI | YYTSRL | QQFSEL | DIQMTQSPSSLSASVGDRVTITCR |
| 3 | AVSGY | GYTE | NWEDY | AVSGYIFSSYWVYWVRQAPGK | SNYIN | YG (SEQ | PRT | ASQDISNYINWYQQKPGKAPKLL |
| | VY | (SEQ ID | (SEQ ID | GLEWVGYINPTTGYTEYADSVK | (SEQ ID | ID | (SEQ ID | IYYTSRLYGGVPSRFSGSGSGTDF |
| | (SEQ ID | NO: 2) | NO: 3) | GRFTISRDNSKNTLYLQMNSLR | NO: 8) | NO: 12) | NO: 10) | TLTISSLQPEDFATYYCQQFSELP |
| | NO: 1) | | | AEDTAVYYCATEGGNWEDYW | | | | RTFGQGTKVEIK (SEQ ID NO: 22) |
| | | | | GQGTLVTVSS (SEQ ID NO: 15) | | | | |
| ADWA16- | IFSSYW | YINPTT | ATEGG | EVQLVESGGGLVQPGGSLRLSC | RASKDI | YYTSRL | QQFSEL | DIQMTQSPSSLSASVGDRVTITCR |
| 4 | AVSGY | GYTE | NWEDY | AVSGYIFSSYWVYWVRQAPGK | SNYIN | YG (SEQ | PRT | ASKDISNYINWYQQKPGKAPKLL |
| | VY | (SEQ ID | (SEQ ID | GLEWVGYINPTTGYTEYADSVK | (SEQ ID | ID | (SEQ ID | IYYTSRLYGGVPSRFSGSGSGTDF |
| | (SEQ ID | NO: 2) | NO: 3) | GRFTISRDNSKNTLYLQMNSLR | NO: 13) | NO: 12) | NO: 10) | TLTISSLQPEDFATYYCQQFSELP |
| | NO: 1) | | | AEDTAVYYCATEGGNWEDYW | | | | RTFGQGTKVEIK (SEQ ID NO: 23) |
| | | | | GQGTLVTVSS (SEQ ID NO: 15) | | | | |
| ADWA16- | IFSSYW | YISPTT | ATEGG | EVQLVESGGGLVQPGGSLRLSC | RASQDI | YYTSRL | QQFSEL | DIQMTQSPSSLSASVGDRVTITCR |
| 3.2 | AVSGY | GYTV | NWEDY | AVSGYIFSSYWVYWVRQAPGK | SNYIN | YG (SEQ | PRT | ASQDISNYINWYQQKPGKAPKLL |
| | VY | (SEQ ID | (SEQ ID | GLEWVGYISPTTGYTVYADSVK | (SEQ ID | ID | (SEQ ID | IYYTSRLYGGVPSRFSGSGSGTDF |
| | (SEQ ID | NO: 4) | NO: 3) | GRFTISRDNSKNTLYLQMNSLR | NO: 8) | NO: 12) | NO: 10) | TLTISSLQPEDFATYYCQQFSELP |
| | NO: 1) | | | AEDTAVYYCATEGGNWEDYW | | | | RTFGQGTKVEIK (SEQ ID NO: 22) |
| | | | | GQGTLVTVSS (SEQ ID NO: 16) | | | | |
| ADWA16 | AASGY | YINPTT | ATEGG | EVQLVESGGGLVQPGGSLRLSC | RASQDI | YYTSRL | QQFSEL | DIQMTQSPSSLSASVGDRVTITCR |
| hugraft | TFSSY | GYTE | NWEDY | AASGYTFSSYWIYWVRQAPGK | SNYLN | YS (SEQ | PRT | ASQDISNYLNWYQQKPGKAPKL |
| | WIY | (SEQ ID | (SEQ ID | GLEWVSYINPTTGYTEYADSVK | (SEQ ID | ID NO: 9) | (SEQ ID | LIYYTSRLYSGVPSRFSGSGSGTD |
| | (SEQ ID | NO: 2) | NO: 3) | GRFTISRDNSKNTLYLQMNSLR | NO: 14) | | NO: 10) | FTLTISSLQPEDFATYYCQQFSEL |
| | NO: 5) | | | AEDTAVYYCATEGGNWEDYW | | | | PRTFGQGTKVEIK (SEQ ID |
| | | | | GQGTLVTVSS (SEQ ID NO: 17) | | | | NO: 24) |
| ADWA16 | TFSSY | YINPTT | ATEGG | QVQLQQSGAELAKPGASMKMS | RASQDI | YYTSRL | QQFSEL | DIQMTQTTSSLSASLGDRVTISCR |
| | KASGY | GYTE | NWEDY | CKASGYTFSSYWIYWVKQRPG | SNYLN | YS (SEQ | PRT | ASQDISNYLNWYQQKPDGTVKL |
| | WIY | (SEQ ID | (SEQ ID | QGLEWIGYINPTTGYTEYNQKF | (SEQ ID | ID NO: 9) | (SEQ ID | LIYYTSRLYSGVPSRFSGSGSGTD |
| | (SEQ ID | NO: 2) | NO: 3) | RDKATLTADKSSNTAYMQLSSL | NO: 14) | | NO: 10) | YSLTISNLEPKDIATYYCQQFSEL |
| | NO: 6) | | | TSEDSAVYYCATEGGNWEDYW | | | | PRTFGGGTKLEIK (SEQ ID |
| | | | | GQGTTLTVSS (SEQ ID NO: 18) | | | | NO: 25) |
| Ab1 | AVSGY | YISPTT | ATEGG | EVQLVESGGGLVQPGGSLRLSC | RASQDI | YYTSRL | QQFSEL | DIQMTQSPSSLSASVGDRVTITCR |
| | IFSSYW | GYTV | NWEDY | AVSGYIFSSYWVYWVRQAPGK | SNYIN | YS (SEQ | PRT | ASQDISNYINWYQQKPGKAPKLL |
| | VY | (SEQ ID | (SEQ ID | GLEWVGYISPTTGYTVYADSVK | (SEQ ID | ID NO: 9) | (SEQ ID | IYYTSRLYSGVPSRFSGSGSGTDF |
| | (SEQ ID | NO: 4) | NO: 3) | GRFTISRDNSKNTLYLQMNSLR | NO: 8) | | NO: 10) | TLTISSLQPEDFATYYCQQFSELP |
| | NO: 1) | | | AEDTAVYYCATEGGNWEDYW | | | | RTFGQGTKVEIK (SEQ ID NO: 20) |
| | | | | GQGTLVTVSS (SEQ ID NO: 16) | | | | |
| Ab2 | IFSSYW | YISPTT | ATEGG | EVQLVESGGGLVQPGGSLRLSC | RASQDI | YYTSRL | QQFSEL | DIQMTQSPSSLSASVGDRVTITCR |
| | AVSGY | GYTE | NWEDY | AVSGYIFSSYWVYWVRQAPGK | SNYIN | YG (SEQ | PRT | ASQDISNYINWYQQKPGKAPKLL |
| | VY | (SEQ ID | (SEQ ID | GLEWVGYISPTTGYTEYADSVK | (SEQ ID | ID | (SEQ ID | IYYTSRLYGGVPSRFSGSGSGTDF |
| | (SEQ ID | NO: 7) | NO: 3) | GRFTISRDNSKNTLYLQMNSLR | NO: 8) | NO: 12) | NO: 10) | TLTISSLQPEDFATYYCQQFSELP |
| | NO: 1) | | | AEDTAVYYCATEGGNWEDYW | | | | RTFGQGTKVEIK (SEQ ID NO: 22) |
| | | | | GQGTLVTVSS (SEQ ID NO: 19) | | | | |
| Ab3 | AVSGY | YISPTT | ATEGG | EVQLVESGGGLVQPGGSLRLSC | RASQDI | YYTSRL | QQFSEL | DIQMTQSPSSLSASVGDRVTITCR |
| | IFSSYW | GYTE | NWEDY | AVSGYIFSSYWVYWVRQAPGK | SNYIN | YS (SEQ | PRT | ASQDISNYINWYQQKPGKAPKLL |
| | VY | (SEQ ID | (SEQ ID | GLEWVGYISPTTGYTEYADSVK | (SEQ ID | ID NO: 9) | (SEQ ID | IYYTSRLYSGVPSRFSGSGSGTDF |
| | (SEQ ID | NO: 7) | NO: 3) | GRFTISRDNSKNTLYLQMNSLR | NO: 8) | | NO: 10) | TLTISSLQPEDFATYYCQQFSELP |
| | NO: 1) | | | AEDTAVYYCATEGGNWEDYW | | | | RTFGQGTKVEIK (SEQ ID NO: 20) |
| | | | | GQGTLVTVSS (SEQ ID NO: 19) | | | | |
| ADWA11 | GFNIK | WIDPD | RLLMD | EVQLQQSGAELVRPGAFVKLSC | RSTKSL | YMSNL | MQSLEY | DIVMTQAAPSVPVTPGESVSISCR |
| | DYYM | NGNTI | Y (SEQ | KASGFNIKDYYMNWVLQRPEQ | LHFNGN | AS (SEQ | PFT | STKSLLHFNGNTYLFWFLQRPGQ |
| | N (SEQ | YDPKF | ID | GLEWIGWIDPDNGNTIYDPKFQ | TYLF | ID | (SEQ ID | SPQRLIYYMSNLASGVPDRFSGR |
| | ID | QG | NO: 53) | GKASITADTSSNTAYLQLSSLTS | (SEQ ID | NO: 55) | NO: 56) | GSGTDFTLRISRVEAEDVGVYYC |
| | NO: 51) | (SEQ ID | | EDTAVYYCARRLLMDYWGQGT | NO: 54) | | | MQSLEYPFTFGTGTKLEIK (SEQ |
| | | NO: 52) | | SVTVSS (SEQ ID NO: 57) | | | | ID NO: 58) |

In some embodiments, an antibody of the disclosure comprises: (1) an HCDR1 having a sequence of any one of SEQ ID NOS:1, 5, and 6 or a variant thereof that has a sequence having one, two, or three amino acid substitutions relative to a sequence of any one of SEQ ID NOS:1, 5, and 6; (2) an HCDR2 having a sequence of any one of SEQ ID NOS:2, 4, and 7 or a variant thereof that has a sequence having one, two, or three amino acid substitutions relative to a sequence of any one of SEQ ID NOS:1, 4, and 7; (3) an HCDR3 having the sequence of SEQ ID NO:3 or a variant thereof that has a sequence having one, two, or three amino acid substitutions relative to the sequence of SEQ ID NO:3; (4) a LCDR1 having a sequence of any one of SEQ ID NOS:8, 11, 13, and 14 or a variant thereof that has a sequence having one, two, or three amino acid substitutions relative to a sequence of any one of SEQ ID NOS:8, 11, 13, and 14; (5) a LCDR2 having a sequence of any one of SEQ ID NOS:9 and 12 or a variant thereof that has a sequence having one or two amino acid substitutions relative to a sequence of any one of SEQ ID NOS:9 and 12; and (6) a LCDR3 having the sequence of SEQ ID NO:10 or a variant thereof that has a sequence having one or two amino acid substitutions relative to the sequence of SEQ ID NO:10.

In some embodiments, an antibody of the disclosure can comprise an HCDR1 having the sequence of SEQ ID NO:1 or a variant thereof that has a sequence having one, two, or three amino acid substitutions relative to the sequence of SEQ ID NO:1, an HCDR2 having the sequence of SEQ ID NO: 4 or a variant thereof that has a sequence having one, two, or three amino acid substitutions relative to the sequence of SEQ ID NO:4, and an HCDR3 having the sequence of SEQ ID NO: 3 or a variant thereof that has a sequence having one, two, or three amino acid substitutions relative to the sequence of SEQ ID NO:3. In some embodiments, an antibody of the disclosure can comprise an HCDR1 having the sequence of SEQ ID NO:1 or a variant thereof that has a sequence having one, two, or three amino acid substitutions relative to the sequence of SEQ ID NO:1, an HCDR2 having the sequence of SEQ ID NO:2 or a variant thereof that has a sequence having one, two, or three amino acid substitutions relative to the sequence of SEQ ID NO:2, and an HCDR3 having the sequence of SEQ ID NO: 3 or a variant thereof that has a sequence having one, two, or three amino acid substitutions relative to the sequence of SEQ ID NO:3. In some embodiments, an antibody of the disclosure can comprise an HCDR1 having the sequence of SEQ ID NO:1 or a variant thereof that has a sequence having one, two, or three amino acid substitutions relative to the sequence of SEQ ID NO:1, an HCDR2 having the sequence of SEQ ID NO: 7 or a variant thereof that has a sequence having one, two, or three amino acid substitutions relative to the sequence of SEQ ID NO:7, and an HCDR3 having the sequence of SEQ ID NO: 3 or a variant thereof that has a sequence having one, two, or three amino acid substitutions relative to the sequence of SEQ ID NO:3. In some embodiments, an antibody of the disclosure can comprise an HCDR1 having the sequence of SEQ ID NO: 5 or a variant thereof that has a sequence having one, two, or three amino acid substitutions relative to the sequence of SEQ ID NO:5, an HCDR2 having the sequence of SEQ ID NO:2 or a variant thereof that has a sequence having one, two, or three amino acid substitutions relative to the sequence of SEQ ID NO:2, and an HCDR3 having the sequence of SEQ ID NO: 3 or a variant thereof that has a sequence having one, two, or three amino acid substitutions relative to the sequence of SEQ ID NO:3. In further embodiments, an antibody of the disclosure can comprise an HCDR1 having the sequence of SEQ ID NO: 6 or a variant thereof that has a sequence having one, two, or three amino acid substitutions relative to the sequence of SEQ ID NO:6, an HCDR2 having the sequence of SEQ ID NO:2 or a variant thereof that has a sequence having one, two, or three amino acid substitutions relative to the sequence of SEQ ID NO:2, and an HCDR3 having the sequence of SEQ ID NO: 3 or a variant thereof that has a sequence having one, two, or three amino acid substitutions relative to the sequence of SEQ ID NO:3.

An antibody of the disclosure can comprise a heavy chain variable region (V_{H}) having an HCDR1, an HCDR2, and an HCDR3 as described herein. In certain embodiments, an antibody of the disclosure can comprise a heavy chain variable region having an HCDR1, an HCDR2, and an HCDR3 of SEQ ID NOS:1-3, respectively, and at least 90% (e.g., 90%, 91%, 92%, 93%, 94%, 95%, 96%, 97%, 98%, 99%, or 100%) identity to the sequence of SEQ ID NO:15. In certain embodiments, an antibody of the disclosure can comprise a heavy chain variable region having an HCDR1, an HCDR2, and an HCDR3 of SEQ ID NOS:1, 4, and 3, respectively, and at least 90% (e.g., 90%, 91%, 92%, 93%, 94%, 95%, 96%, 97%, 98%, 99%, or 100%) identity to the sequence of SEQ ID NO: 16. In certain embodiments, an antibody of the disclosure can comprise a heavy chain variable region having an HCDR1, an HCDR2, and an HCDR3 of SEQ ID NOS:5, 2, and 3, respectively, and at least 90% (e.g., 90%, 91%, 92%, 93%, 94%, 95%, 96%, 97%, 98%, 99%, or 100%) identity to the sequence of SEQ ID NO:17. In certain embodiments, an antibody of the disclosure can comprise a heavy chain variable region having an HCDR1, an HCDR2, and an HCDR3 of SEQ ID NOS:6, 2, and 3, respectively, and at least 90% (e.g., 90%, 91%, 92%, 93%, 94%, 95%, 96%, 97%, 98%, 99%, or 100%) identity to the sequence of SEQ ID NO: 18. In certain embodiments, an antibody of the disclosure can comprise a heavy chain variable region having an HCDR1, an HCDR2, and an HCDR3 of SEQ ID NOS:1, 7, and 3, respectively, and at least 90% (e.g., 90%, 91%, 92%, 93%, 94%, 95%, 96%, 97%, 98%, 99%, or 100%) identity to the sequence of SEQ ID NO:19.

In some embodiments, an antibody of the disclosure can comprise an LCDR1 having the sequence of SEQ ID NO: 8 or a variant thereof that has a sequence having one, two, or three amino acid substitutions relative to the sequence of SEQ ID NO:8, an LCDR2 having the sequence of SEQ ID NO: 12 or a variant thereof that has a sequence having one or two amino acid substitutions relative to the sequence of SEQ ID NO:12, and an LCDR3 having the sequence of SEQ ID NO: 10 or a variant thereof that has a sequence having one or two amino acid substitutions relative to the sequence of SEQ ID NO: 10. In some embodiments, an antibody of the disclosure can comprise a LCDR1 having the sequence of SEQ ID NO: 8 or a variant thereof that has a sequence having one, two, or three amino acid substitutions relative to the sequence of SEQ ID NO:8, a LCDR2 having the sequence of SEQ ID NO: 9 or a variant thereof that has a sequence having one or two amino acid substitutions relative to the sequence of SEQ ID NO:9, and a LCDR3 having the sequence of SEQ ID NO: 10 or a variant thereof that has a sequence having one or two amino acid substitutions relative to the sequence of SEQ ID NO:10. In some embodiments, an antibody of the disclosure can comprise a LCDR1 having the sequence of SEQ ID NO: 11 or a variant thereof that has a sequence having one, two, or three amino acid substitutions relative to the sequence of SEQ ID NO:11, a LCDR2 having the sequence of SEQ ID NO: 9 or a variant thereof that has a sequence having one or two amino acid substitutions relative to the sequence of SEQ ID NO:9, and a LCDR3 having the sequence of SEQ ID NO: 10 or a variant thereof that has a sequence having one or two amino acid substitutions relative to the sequence of SEQ ID NO:10.

In some embodiments, an antibody of the disclosure can comprise a LCDR1 having the sequence of SEQ ID NO: 13 or a variant thereof that has a sequence having one, two, or three amino acid substitutions relative to the sequence of SEQ ID NO:13, a LCDR2 having the sequence of SEQ ID NO: 12 or a variant thereof that has a sequence having one or two amino acid substitutions relative to the sequence of SEQ ID NO:12, and a LCDR3 having the sequence of SEQ ID NO: 10 or a variant thereof that has a sequence having one or two amino acid substitutions relative to the sequence of SEQ ID NO: 10. In some embodiments, an antibody of the disclosure can comprise a LCDR1 having the sequence of SEQ ID NO: 14 or a variant thereof that has a sequence having one, two, or three amino acid substitutions relative to the sequence of SEQ ID NO: 14, a LCDR2 having the sequence of SEQ ID NO: 9 or a variant thereof that has a sequence having one or two amino acid substitutions relative to the sequence of SEQ ID NO:9, and a LCDR3 having the sequence of SEQ ID NO: 10 or a variant thereof that has a sequence having one or two amino acid substitutions relative to the sequence of SEQ ID NO:10.

An antibody of the disclosure can comprise a light chain variable region (V_{L}) having a LCDR1, a LCDR2, and a LCDR3 as described herein. In certain embodiments, an antibody of the disclosure can comprise a light chain variable region having a LCDR1, a LCDR2, and a LCDR3 of SEQ ID NOS:8-10, respectively, and at least 90% (e.g., 90%, 91%, 92%, 93%, 94%, 95%, 96%, 97%, 98%, 99%, or 100%) identity to the sequence of SEQ ID NO:20. In certain embodiments, an antibody of the disclosure can comprise a light chain variable region having a LCDR1, a LCDR2, and a LCDR3 of SEQ ID NOS:11, 9, and 10, respectively, and at least 90% (e.g., 90%, 91%, 92%, 93%, 94%, 95%, 96%, 97%, 98%, 99%, or 100%) identity to the sequence of SEQ ID NO:21. In certain embodiments, an antibody of the disclosure can comprise a light chain variable region having a LCDR1, a LCDR2, and a LCDR3 of SEQ ID NOS:8, 12, and 10, respectively, and at least 90% (e.g., 90%, 91%, 92%, 93%, 94%, 95%, 96%, 97%, 98%, 99%, or 100%) identity to the sequence of SEQ ID NO:22. In certain embodiments, an antibody of the disclosure can comprise a light chain variable region having a LCDR1, a LCDR2, and a LCDR3 of SEQ ID NOS:13, 12, and 10, respectively, and at least 90% (e.g., 90%, 91%, 92%, 93%, 94%, 95%, 96%, 97%, 98%, 99%, or 100%) identity to the sequence of SEQ ID NO:23. In certain embodiments, an antibody of the disclosure can comprise a light chain variable region having a LCDR1, a LCDR2, and a LCDR3 of SEQ ID NOS:14, 9, and 10, respectively, and at least 90% (e.g., 90%, 91%, 92%, 93%, 94%, 95%, 96%, 97%, 98%, 99%, or 100%) identity to the sequence of SEQ ID NO:24. In certain embodiments, an antibody of the disclosure can comprise a light chain variable region having a LCDR1, a LCDR2, and a LCDR3 of SEQ ID NOS:14, 9, and 10, respectively, and at least 90% (e.g., 90%, 91%, 92%, 93%, 94%, 95%, 96%, 97%, 98%, 99%, or 100%) identity to the sequence of SEQ ID NO:25.

### ADWA16-1

In particular embodiments, an antibody of the disclosure can comprise: (1) an HCDR1 having the sequence of SEQ ID NO:1; (2) an HCDR2 having the sequence of SEQ ID NO:2; (3) an HCDR3 having the sequence of SEQ ID NO:3; (4) a LCDR1 having the sequence of SEQ ID NO:8; (5) a LCDR2 having the sequence of SEQ ID NO:9; and (6) a LCDR3 having the sequence of SEQ ID NO:10. In some embodiments, the antibody can comprise (1) a heavy chain variable region having an HCDR1, an HCDR2, and an HCDR3 of SEQ ID NOS:1-3, respectively, and at least 90% (e.g., 90%, 91%, 92%, 93%, 94%, 95%, 96%, 97%, 98%, 99%, or 100%) identity to the sequence of SEQ ID NO: 15, and (2) a light chain variable region having a LCDR1, a LCDR2, and a LCDR3 of SEQ ID NOS:8-10, respectively, and at least 90% (e.g., 90%, 91%, 92%, 93%, 94%, 95%, 96%, 97%, 98%, 99%, or 100%) identity to the sequence of SEQ ID NO: 20. Such an antibody can be an IgG1, IgG2, IgG3, or IgG4.

In certain embodiments, the antibody comprises a heavy chain having at least 90%
(e.g., 90%, 91%, 92%, 93%, 94%, 95%, 96%, 97%, 98%, 99%, or 100%) identity to the
sequence of SEQ ID NO: 26:
EVQLVESGGGLVQPGGSLRLSCAVSGYIFSSYWVYWVRQAPGKGLEWVGYINPTTG
YTEYADSVKGRFTISRDNSKNTLYLQMNSLRAEDTAVYYCATEGGNWEDYWGQGT
LVTVSSASTKGPSVFPLAPSSKSTSGGTAALGCLVKDYFPEPVTVSWNSGALTSGVH
TFPAVLQSSGLYSLSSVVTVPSSSLGTQTYICNVNHKPSNTKVDKRVEPKSCDKTHTC
PPCPAPELLGGPSVFLFPPKPKDTLMISRTPEVTCVVVDVSHEDPEVKFNWYVDGVE
VHNAKTKPREEQYNSTYRVVSVLTVLHQDWLNGKEYKCKVSNKALPAPIEKTISKA
KGQPREPQVYTLPPSREEMTKNQVSLTCLVKGFYPSDIAVEWESNGQPENNYKTTPP
VLDSDGSFFLYSKLTVDKSRWQQGNVFSCSVMHEALHNHYTQKSLSLSPGK,
and
a light chain having at least 90% (e.g., 90%, 91%, 92%, 93%, 94%, 95%, 96%, 97%,
98%, 99%, or 100%) identity to the sequence of SEQ ID NO: 27:
DIQMTQSPSSLSASVGDRVTITCRASQDISNYINWYQQKPGKAPKLLIYYTSRLYSGV
PSRFSGSGSGTDFTLTISSLQPEDFATYYCQQFSELPRTFGQGTKVEIKRTVAAPSVFIF
PPSDEQLKSGTASVVCLLNNFYPREAKVQWKVDNALQSGNSQESVTEQDSKDSTYS
LSSTLTLSKADYEKHKVYACEVTHQGLSSPVTKSFNRGEC.
In certain embodiments, the antibody comprises a heavy chain having at least 90%
(e.g., 90%, 91%, 92%, 93%, 94%, 95%, 96%, 97%, 98%, 99%, or 100%) identity to the
sequence of SEQ ID NO: 28:
EVQLVESGGGLVQPGGSLRLSCAVSGYIFSSYWVYWVRQAPGKGLEWVGYINPTTG
YTEYADSVKGRFTISRDNSKNTLYLQMNSLRAEDTAVYYCATEGGNWEDYWGQGT
LVTVSSASTKGPSVFPLAPSSKSTSGGTAALGCLVKDYFPEPVTVSWNSGALTSGVH
TFPAVLQSSGLYSLSSVVTVPSSSLGTQTYICNVNHKPSNTKVDKRVEPKSCDKTHTC
PPCPAPEAAGGPSVFLFPPKPKDTLMISRTPEVTCVVVDVSHEDPEVKFNWYVDGVE
VHNAKTKPREEQYNSTYRVVSVLTVLHQDWLNGKEYKCKVSNKALPAPIEKTISKA
KGQPREPQVYTLPPSREEMTKNQVSLTCLVKGFYPSDIAVEWESNGQPENNYKTTPP
VLDSDGSFFLYSKLTVDKSRWQQGNVFSCSVMHEALHNHYTQKSLSLSPGK,
and
a light chain having at least 90% (e.g., 90%, 91%, 92%, 93%, 94%, 95%, 96%, 97%,
98%, 99%, or 100%) identity to the sequence of SEQ ID NO: 27:
DIQMTQSPSSLSASVGDRVTITCRASQDISNYINWYQQKPGKAPKLLIYYTSRLYSGV
PSRFSGSGSGTDFTLTISSLQPEDFATYYCQQFSELPRTFGQGTKVEIKRTVAAPSVFIF
PPSDEQLKSGTASVVCLLNNFYPREAKVQWKVDNALQSGNSQESVTEQDSKDSTYS
LSSTLTLSKADYEKHKVYACEVTHQGLSSPVTKSFNRGEC.
In certain embodiments, the antibody comprises a heavy chain having at least 90%
(e.g., 90%, 91%, 92%, 93%, 94%, 95%, 96%, 97%, 98%, 99%, or 100%) identity to the
sequence of SEQ ID NO: 29:
EVQLVESGGGLVQPGGSLRLSCAVSGYIFSSYWVYWVRQAPGKGLEWVGYINPTTG
YTEYADSVKGRFTISRDNSKNTLYLQMNSLRAEDTAVYYCATEGGNWEDYWGQGT
LVTVSSASTKGPSVFPLAPSSKSTSGGTAALGCLVKDYFPEPVTVSWNSGALTSGVH
TFPAVLQSSGLYSLSSVVTVPSSSLGTQTYICNVNHKPSNTKVDKRVEPKSCDKTHTC
PPCPAPELLGGPSVFLFPPKPKDTLMISRTPEVTCVVVDVSHEDPEVKFNWYVDGVE
VHNAKTKPREEQYASTYRVVSVLTVLHQDWLNGKEYKCKVSNKALPAPIEKTISKA
KGQPREPQVYTLPPSREEMTKNQVSLTCLVKGFYPSDIAVEWESNGQPENNYKTTPP
VLDSDGSFFLYSKLTVDKSRWQQGNVFSCSVMHEALHNHYTQKSLSLSPGK,
and
a light chain having at least 90% (e.g., 90%, 91%, 92%, 93%, 94%, 95%, 96%, 97%,
98%, 99%, or 100%) identity to the sequence of SEQ ID NO: 27:
DIQMTQSPSSLSASVGDRVTITCRASQDISNYINWYQQKPGKAPKLLIYYTSRLYSGV
PSRFSGSGSGTDFTLTISSLQPEDFATYYCQQFSELPRTFGQGTKVEIKRTVAAPSVFIF
PPSDEQLKSGTASVVCLLNNFYPREAKVQWKVDNALQSGNSQESVTEQDSKDSTYS
LSSTLTLSKADYEKHKVYACEVTHQGLSSPVTKSFNRGEC.

### ADWA16-2

In particular embodiments, an antibody of the disclosure can comprise: (1) an HCDR1 having the sequence of SEQ ID NO:1; (2) an HCDR2 having the sequence of SEQ ID NO:2; (3) an HCDR3 having the sequence of SEQ ID NO:3; (4) a LCDR1 having the sequence of SEQ ID NO: 11; (5) a LCDR2 having the sequence of SEQ ID NO: 9; and (6) a LCDR3 having the sequence of SEQ ID NO: 10. In some embodiments, the antibody can comprise (1) a heavy chain variable region having an HCDR1, an HCDR2, and an HCDR3 of SEQ ID NOS:1-3, respectively, and at least 90% (e.g., 90%, 91%, 92%, 93%, 94%, 95%, 96%, 97%, 98%, 99%, or 100%) identity to the sequence of SEQ ID NO: 15, and (2) a light chain variable region having a LCDR1, a LCDR2, and a LCDR3 of SEQ ID NOS:11, 9, and 10, respectively, and at least 90% (e.g., 90%, 91%, 92%, 93%, 94%, 95%, 96%, 97%, 98%, 99%, or 100%) identity to the sequence of SEQ ID NO: 21. Such an antibody can be an IgG1, IgG2, IgG3, or IgG4.

In certain embodiments, the antibody comprises a heavy chain having at least 90%
(e.g., 90%, 91%, 92%, 93%, 94%, 95%, 96%, 97%, 98%, 99%, or 100%) identity to the
sequence of SEQ ID NO: 26:
EVQLVESGGGLVQPGGSLRLSCAVSGYIFSSYWVYWVRQAPGKGLEWVGYINPTTG
YTEYADSVKGRFTISRDNSKNTLYLQMNSLRAEDTAVYYCATEGGNWEDYWGQGT
LVTVSSASTKGPSVFPLAPSSKSTSGGTAALGCLVKDYFPEPVTVSWNSGALTSGVH
TFPAVLQSSGLYSLSSVVTVPSSSLGTQTYICNVNHKPSNTKVDKRVEPKSCDKTHTC
PPCPAPELLGGPSVFLFPPKPKDTLMISRTPEVTCVVVDVSHEDPEVKFNWYVDGVE
VHNAKTKPREEQYNSTYRVVSVLTVLHQDWLNGKEYKCKVSNKALPAPIEKTISKA
KGQPREPQVYTLPPSREEMTKNQVSLTCLVKGFYPSDIAVEWESNGQPENNYKTTPP
VLDSDGSFFLYSKLTVDKSRWQQGNVFSCSVMHEALHNHYTQKSLSLSPGK,
and
a light chain having at least 90% (e.g., 90%, 91%, 92%, 93%, 94%, 95%, 96%, 97%,
98%, 99%, or 100%) identity to the sequence of SEQ ID NO: 30:
DIQMTQSPSSLSASVGDRVTITCRASKDISNYLNWYQQKPGKAPKLLIYYTSRLYSGV
PSRFSGSGSGTDFTLTISSLQPEDFATYYCQQFSELPRTFGQGTKVEIKRTVAAPSVFIF
PPSDEQLKSGTASVVCLLNNFYPREAKVQWKVDNALQSGNSQESVTEQDSKDSTYS
LSSTLTLSKADYEKHKVYACEVTHQGLSSPVTKSFNRGEC.
In certain embodiments, the antibody comprises a heavy chain having at least 90%
(e.g., 90%, 91%, 92%, 93%, 94%, 95%, 96%, 97%, 98%, 99%, or 100%) identity to the
sequence of SEQ ID NO: 28:
EVQLVESGGGLVQPGGSLRLSCAVSGYIFSSYWVYWVRQAPGKGLEWVGYINPTTG
YTEYADSVKGRFTISRDNSKNTLYLQMNSLRAEDTAVYYCATEGGNWEDYWGQGT
LVTVSSASTKGPSVFPLAPSSKSTSGGTAALGCLVKDYFPEPVTVSWNSGALTSGVH
TFPAVLQSSGLYSLSSVVTVPSSSLGTQTYICNVNHKPSNTKVDKRVEPKSCDKTHTC
PPCPAPEAAGGPSVFLFPPKPKDTLMISRTPEVTCVVVDVSHEDPEVKFNWYVDGVE
VHNAKTKPREEQYNSTYRVVSVLTVLHQDWLNGKEYKCKVSNKALPAPIEKTISKA
KGQPREPQVYTLPPSREEMTKNQVSLTCLVKGFYPSDIAVEWESNGQPENNYKTTPP
VLDSDGSFFLYSKLTVDKSRWQQGNVFSCSVMHEALHNHYTQKSLSLSPGK,
and
a light chain having at least 90% (e.g., 90%, 91%, 92%, 93%, 94%, 95%, 96%, 97%,
98%, 99%, or 100%) identity to the sequence of SEQ ID NO: 30:
DIQMTQSPSSLSASVGDRVTITCRASKDISNYLNWYQQKPGKAPKLLIYYTSRLYSGV
PSRFSGSGSGTDFTLTISSLQPEDFATYYCQQFSELPRTFGQGTKVEIKRTVAAPSVFIF
PPSDEQLKSGTASVVCLLNNFYPREAKVQWKVDNALQSGNSQESVTEQDSKDSTYS
LSSTLTLSKADYEKHKVYACEVTHQGLSSPVTKSFNRGEC.
In certain embodiments, the antibody comprises a heavy chain having at least 90%
(e.g., 90%, 91%, 92%, 93%, 94%, 95%, 96%, 97%, 98%, 99%, or 100%) identity to the
sequence of SEQ ID NO: 29:
EVQLVESGGGLVQPGGSLRLSCAVSGYIFSSYWVYWVRQAPGKGLEWVGYINPTTG
YTEYADSVKGRFTISRDNSKNTLYLQMNSLRAEDTAVYYCATEGGNWEDYWGQGT
LVTVSSASTKGPSVFPLAPSSKSTSGGTAALGCLVKDYFPEPVTVSWNSGALTSGVH
TFPAVLQSSGLYSLSSVVTVPSSSLGTQTYICNVNHKPSNTKVDKRVEPKSCDKTHTC
PPCPAPELLGGPSVFLFPPKPKDTLMISRTPEVTCVVVDVSHEDPEVKFNWYVDGVE
VHNAKTKPREEQYASTYRVVSVLTVLHQDWLNGKEYKCKVSNKALPAPIEKTISKA
KGQPREPQVYTLPPSREEMTKNQVSLTCLVKGFYPSDIAVEWESNGQPENNYKTTPP
VLDSDGSFFLYSKLTVDKSRWQQGNVFSCSVMHEALHNHYTQKSLSLSPGK,
and
a light chain having at least 90% (e.g., 90%, 91%, 92%, 93%, 94%, 95%, 96%, 97%,
98%, 99%, or 100%) identity to the sequence of SEQ ID NO: 30:
DIQMTQSPSSLSASVGDRVTITCRASKDISNYLNWYQQKPGKAPKLLIYYTSRLYSGV
PSRFSGSGSGTDFTLTISSLQPEDFATYYCQQFSELPRTFGQGTKVEIKRTVAAPSVFIF
PPSDEQLKSGTASVVCLLNNFYPREAKVQWKVDNALQSGNSQESVTEQDSKDSTYS
LSSTLTLSKADYEKHKVYACEVTHQGLSSPVTKSFNRGEC.

### ADWA16-3

In particular embodiments, an antibody of the disclosure can comprise: (1) an HCDR1 having the sequence of SEQ ID NO:1; (2) an HCDR2 having the sequence of SEQ ID NO:2; (3) an HCDR3 having the sequence of SEQ ID NO:3; (4) a LCDR1 having the sequence of SEQ ID NO: 8; (5) a LCDR2 having the sequence of SEQ ID NO: 12; and (6) a LCDR3 having the sequence of SEQ ID NO: 10. In some embodiments, the antibody can comprise (1) a heavy chain variable region having an HCDR1, an HCDR2, and an HCDR3 of SEQ ID NOS:1-3, respectively, and at least 90% (e.g., 90%, 91%, 92%, 93%, 94%, 95%, 96%, 97%, 98%, 99%, or 100%) identity to the sequence of SEQ ID NO: 15, and (2) a light chain variable region having a LCDR1, a LCDR2, and a LCDR3 of SEQ ID NOS:8, 12, and 10, respectively, and at least 90% (e.g., 90%, 91%, 92%, 93%, 94%, 95%, 96%, 97%, 98%, 99%, or 100%) identity to the sequence of SEQ ID NO: 22. Such an antibody can be an IgG1, IgG2, IgG3, or IgG4.

In certain embodiments, the antibody comprises a heavy chain having at least 90%
(e.g., 90%, 91%, 92%, 93%, 94%, 95%, 96%, 97%, 98%, 99%, or 100%) identity to the
sequence of SEQ ID NO: 26:
EVQLVESGGGLVQPGGSLRLSCAVSGYIFSSYWVYWVRQAPGKGLEWVGYINPTTG
YTEYADSVKGRFTISRDNSKNTLYLQMNSLRAEDTAVYYCATEGGNWEDYWGQGT
LVTVSSASTKGPSVFPLAPSSKSTSGGTAALGCLVKDYFPEPVTVSWNSGALTSGVH
TFPAVLQSSGLYSLSSVVTVPSSSLGTQTYICNVNHKPSNTKVDKRVEPKSCDKTHTC
PPCPAPELLGGPSVFLFPPKPKDTLMISRTPEVTCVVVDVSHEDPEVKFNWYVDGVE
VHNAKTKPREEQYNSTYRVVSVLTVLHQDWLNGKEYKCKVSNKALPAPIEKTISKA
KGQPREPQVYTLPPSREEMTKNQVSLTCLVKGFYPSDIAVEWESNGQPENNYKTTPP
VLDSDGSFFLYSKLTVDKSRWQQGNVFSCSVMHEALHNHYTQKSLSLSPGK,
and
a light chain having at least 90% (e.g., 90%, 91%, 92%, 93%, 94%, 95%, 96%, 97%,
98%, 99%, or 100%) identity to the sequence of SEQ ID NO: 31:
DIQMTQSPSSLSASVGDRVTITCRASQDISNYINWYQQKPGKAPKLLIYYTSRLYGGV
PSRFSGSGSGTDFTLTISSLQPEDFATYYCQQFSELPRTFGQGTKVEIKRTVAAPSVFIF
PPSDEQLKSGTASVVCLLNNFYPREAKVQWKVDNALQSGNSQESVTEQDSKDSTYS
LSSTLTLSKADYEKHKVYACEVTHQGLSSPVTKSFNRGEC.
In certain embodiments, the antibody comprises a heavy chain having at least 90%
(e.g., 90%, 91%, 92%, 93%, 94%, 95%, 96%, 97%, 98%, 99%, or 100%) identity to the
sequence of SEQ ID NO: 28:
EVQLVESGGGLVQPGGSLRLSCAVSGYIFSSYWVYWVRQAPGKGLEWVGYINPTTG
YTEYADSVKGRFTISRDNSKNTLYLQMNSLRAEDTAVYYCATEGGNWEDYWGQGT
LVTVSSASTKGPSVFPLAPSSKSTSGGTAALGCLVKDYFPEPVTVSWNSGALTSGVH
TFPAVLQSSGLYSLSSVVTVPSSSLGTQTYICNVNHKPSNTKVDKRVEPKSCDKTHTC
PPCPAPEAAGGPSVFLFPPKPKDTLMISRTPEVTCVVVDVSHEDPEVKFNWYVDGVE
VHNAKTKPREEQYNSTYRVVSVLTVLHQDWLNGKEYKCKVSNKALPAPIEKTISKA
KGQPREPQVYTLPPSREEMTKNQVSLTCLVKGFYPSDIAVEWESNGQPENNYKTTPP
VLDSDGSFFLYSKLTVDKSRWQQGNVFSCSVMHEALHNHYTQKSLSLSPGK,
and
a light chain having at least 90% (e.g., 90%, 91%, 92%, 93%, 94%, 95%, 96%, 97%,
98%, 99%, or 100%) identity to the sequence of SEQ ID NO: 31:
DIQMTQSPSSLSASVGDRVTITCRASQDISNYINWYQQKPGKAPKLLIYYTSRLYGGV
PSRFSGSGSGTDFTLTISSLQPEDFATYYCQQFSELPRTFGQGTKVEIKRTVAAPSVFIF
PPSDEQLKSGTASVVCLLNNFYPREAKVQWKVDNALQSGNSQESVTEQDSKDSTYS
LSSTLTLSKADYEKHKVYACEVTHQGLSSPVTKSFNRGEC.
In certain embodiments, the antibody comprises a heavy chain having at least 90%
(e.g., 90%, 91%, 92%, 93%, 94%, 95%, 96%, 97%, 98%, 99%, or 100%) identity to the
sequence of SEQ ID NO: 29:
EVQLVESGGGLVQPGGSLRLSCAVSGYIFSSYWVYWVRQAPGKGLEWVGYINPTTG
YTEYADSVKGRFTISRDNSKNTLYLQMNSLRAEDTAVYYCATEGGNWEDYWGQGT
LVTVSSASTKGPSVFPLAPSSKSTSGGTAALGCLVKDYFPEPVTVSWNSGALTSGVH
TFPAVLQSSGLYSLSSVVTVPSSSLGTQTYICNVNHKPSNTKVDKRVEPKSCDKTHTC
PPCPAPELLGGPSVFLFPPKPKDTLMISRTPEVTCVVVDVSHEDPEVKFNWYVDGVE
VHNAKTKPREEQYASTYRVVSVLTVLHQDWLNGKEYKCKVSNKALPAPIEKTISKA
KGQPREPQVYTLPPSREEMTKNQVSLTCLVKGFYPSDIAVEWESNGQPENNYKTTPP
VLDSDGSFFLYSKLTVDKSRWQQGNVFSCSVMHEALHNHYTQKSLSLSPGK,
and
a light chain having at least 90% (e.g., 90%, 91%, 92%, 93%, 94%, 95%, 96%, 97%,
98%, 99%, or 100%) identity to the sequence of SEQ ID NO: 31:
DIQMTQSPSSLSASVGDRVTITCRASQDISNYINWYQQKPGKAPKLLIYYTSRLYGGV
PSRFSGSGSGTDFTLTISSLQPEDFATYYCQQFSELPRTFGQGTKVEIKRTVAAPSVFIF
PPSDEQLKSGTASVVCLLNNFYPREAKVQWKVDNALQSGNSQESVTEQDSKDSTYS
LSSTLTLSKADYEKHKVYACEVTHQGLSSPVTKSFNRGEC.

### ADWA16-4

In particular embodiments, an antibody of the disclosure can comprise: (1) an HCDR1 having the sequence of SEQ ID NO:1; (2) an HCDR2 having the sequence of SEQ ID NO:2; (3) an HCDR3 having the sequence of SEQ ID NO:3; (4) a LCDR1 having the sequence of SEQ ID NO: 13; (5) a LCDR2 having the sequence of SEQ ID NO: 12; and (6) a LCDR3 having the sequence of SEQ ID NO: 10. In some embodiments, the antibody can comprise (1) a heavy chain variable region having an HCDR1, an HCDR2, and an HCDR3 of SEQ ID NOS:1-3, respectively, and at least 90% (e.g., 90%, 91%, 92%, 93%, 94%, 95%, 96%, 97%, 98%, 99%, or 100%) identity to the sequence of SEQ ID NO: 15, and (2) a light chain variable region having a LCDR1, a LCDR2, and a LCDR3 of SEQ ID NOS:13, 12, and 10, respectively, and at least 90% (e.g., 90%, 91%, 92%, 93%, 94%, 95%, 96%, 97%, 98%, 99%, or 100%) identity to the sequence of SEQ ID NO: 23. Such an antibody can be an IgG1, IgG2, IgG3, or IgG4.

In certain embodiments, the antibody comprises a heavy chain having at least 90%
(e.g., 90%, 91%, 92%, 93%, 94%, 95%, 96%, 97%, 98%, 99%, or 100%) identity to the
sequence of SEQ ID NO: 26:
EVQLVESGGGLVQPGGSLRLSCAVSGYIFSSYWVYWVRQAPGKGLEWVGYINPTTG
YTEYADSVKGRFTISRDNSKNTLYLQMNSLRAEDTAVYYCATEGGNWEDYWGQGT
LVTVSSASTKGPSVFPLAPSSKSTSGGTAALGCLVKDYFPEPVTVSWNSGALTSGVH
TFPAVLQSSGLYSLSSVVTVPSSSLGTQTYICNVNHKPSNTKVDKRVEPKSCDKTHTC
PPCPAPELLGGPSVFLFPPKPKDTLMISRTPEVTCVVVDVSHEDPEVKFNWYVDGVE
VHNAKTKPREEQYNSTYRVVSVLTVLHQDWLNGKEYKCKVSNKALPAPIEKTISKA
KGQPREPQVYTLPPSREEMTKNQVSLTCLVKGFYPSDIAVEWESNGQPENNYKTTPP
VLDSDGSFFLYSKLTVDKSRWQQGNVFSCSVMHEALHNHYTQKSLSLSPGK,
and
a light chain having at least 90% (e.g., 90%, 91%, 92%, 93%, 94%, 95%, 96%, 97%,
98%, 99%, or 100%) identity to the sequence of SEQ ID NO: 32:
DIQMTQSPSSLSASVGDRVTITCRASKDISNYINWYQQKPGKAPKLLIYYTSRLYGGV
PSRFSGSGSGTDFTLTISSLQPEDFATYYCQQFSELPRTFGQGTKVEIKRTVAAPSVFIF
PPSDEQLKSGTASVVCLLNNFYPREAKVQWKVDNALQSGNSQESVTEQDSKDSTYS
LSSTLTLSKADYEKHKVYACEVTHQGLSSPVTKSFNRGEC.
In certain embodiments, the antibody comprises a heavy chain having at least 90%
(e.g., 90%, 91%, 92%, 93%, 94%, 95%, 96%, 97%, 98%, 99%, or 100%) identity to the
sequence of SEQ ID NO: 28:
EVQLVESGGGLVQPGGSLRLSCAVSGYIFSSYWVYWVRQAPGKGLEWVGYINPTTG
YTEYADSVKGRFTISRDNSKNTLYLQMNSLRAEDTAVYYCATEGGNWEDYWGQGT
LVTVSSASTKGPSVFPLAPSSKSTSGGTAALGCLVKDYFPEPVTVSWNSGALTSGVH
TFPAVLQSSGLYSLSSVVTVPSSSLGTQTYICNVNHKPSNTKVDKRVEPKSCDKTHTC
PPCPAPEAAGGPSVFLFPPKPKDTLMISRTPEVTCVVVDVSHEDPEVKFNWYVDGVE
VHNAKTKPREEQYNSTYRVVSVLTVLHQDWLNGKEYKCKVSNKALPAPIEKTISKA
KGQPREPQVYTLPPSREEMTKNQVSLTCLVKGFYPSDIAVEWESNGQPENNYKTTPP
VLDSDGSFFLYSKLTVDKSRWQQGNVFSCSVMHEALHNHYTQKSLSLSPGK,
and
a light chain having at least 90% (e.g., 90%, 91%, 92%, 93%, 94%, 95%, 96%, 97%,
98%, 99%, or 100%) identity to the sequence of SEQ ID NO: 32:
DIQMTQSPSSLSASVGDRVTITCRASKDISNYINWYQQKPGKAPKLLIYYTSRLYGGV
PSRFSGSGSGTDFTLTISSLQPEDFATYYCQQFSELPRTFGQGTKVEIKRTVAAPSVFIF
PPSDEQLKSGTASVVCLLNNFYPREAKVQWKVDNALQSGNSQESVTEQDSKDSTYS
LSSTLTLSKADYEKHKVYACEVTHQGLSSPVTKSFNRGEC.
In certain embodiments, the antibody comprises a heavy chain having at least 90%
(e.g., 90%, 91%, 92%, 93%, 94%, 95%, 96%, 97%, 98%, 99%, or 100%) identity to the
sequence of SEQ ID NO: 29:
EVQLVESGGGLVQPGGSLRLSCAVSGYIFSSYWVYWVRQAPGKGLEWVGYINPTTG
YTEYADSVKGRFTISRDNSKNTLYLQMNSLRAEDTAVYYCATEGGNWEDYWGQGT
LVTVSSASTKGPSVFPLAPSSKSTSGGTAALGCLVKDYFPEPVTVSWNSGALTSGVH
TFPAVLQSSGLYSLSSVVTVPSSSLGTQTYICNVNHKPSNTKVDKRVEPKSCDKTHTC
PPCPAPELLGGPSVFLFPPKPKDTLMISRTPEVTCVVVDVSHEDPEVKFNWYVDGVE
VHNAKTKPREEQYASTYRVVSVLTVLHQDWLNGKEYKCKVSNKALPAPIEKTISKA
KGQPREPQVYTLPPSREEMTKNQVSLTCLVKGFYPSDIAVEWESNGQPENNYKTTPP
VLDSDGSFFLYSKLTVDKSRWQQGNVFSCSVMHEALHNHYTQKSLSLSPGK,
and
a light chain having at least 90% (e.g., 90%, 91%, 92%, 93%, 94%, 95%, 96%, 97%,
98%, 99%, or 100%) identity to the sequence of SEQ ID NO: 32:
DIQMTQSPSSLSASVGDRVTITCRASKDISNYINWYQQKPGKAPKLLIYYTSRLYGGV
PSRFSGSGSGTDFTLTISSLQPEDFATYYCQQFSELPRTFGQGTKVEIKRTVAAPSVFIF
PPSDEQLKSGTASVVCLLNNFYPREAKVQWKVDNALQSGNSQESVTEQDSKDSTYS
LSSTLTLSKADYEKHKVYACEVTHQGLSSPVTKSFNRGEC.

### ADWA16-3.2

In particular embodiments, an antibody of the disclosure can comprise: (1) an HCDR1 having the sequence of SEQ ID NO:1; (2) an HCDR2 having the sequence of SEQ ID NO: 4; (3) an HCDR3 having the sequence of SEQ ID NO: 3; (4) a LCDR1 having the sequence of SEQ ID NO: 8; (5) a LCDR2 having the sequence of SEQ ID NO: 12; and (6) a LCDR3 having the sequence of SEQ ID NO: 10. In some embodiments, the antibody can comprise (1) a heavy chain variable region having an HCDR1, an HCDR2, and an HCDR3 of SEQ ID NOS:1, 4, and 3, respectively, and at least 90% (e.g., 90%, 91%, 92%, 93%, 94%, 95%, 96%, 97%, 98%, 99%, or 100%) identity to the sequence of SEQ ID NO: 16, and (2) a light chain variable region having a LCDR1, a LCDR2, and a LCDR3 of SEQ ID NOS:8, 12, and 10, respectively, and at least 90% (e.g., 90%, 91%, 92%, 93%, 94%, 95%, 96%, 97%, 98%, 99%, or 100%) identity to the sequence of SEQ ID NO: 22. Such an antibody can be an IgG1, IgG2, IgG3, or IgG4.

In certain embodiments, the antibody comprises a heavy chain having at least 90%
(e.g., 90%, 91%, 92%, 93%, 94%, 95%, 96%, 97%, 98%, 99%, or 100%) identity to the
sequence of SEQ ID NO: 33:
EVQLVESGGGLVQPGGSLRLSCAVSGYIFSSYWVYWVRQAPGKGLEWVGYISPTTG
YTVYADSVKGRFTISRDNSKNTLYLQMNSLRAEDTAVYYCATEGGNWEDYWGQGT
LVTVSSASTKGPSVFPLAPSSKSTSGGTAALGCLVKDYFPEPVTVSWNSGALTSGVH
TFPAVLQSSGLYSLSSVVTVPSSSLGTQTYICNVNHKPSNTKVDKRVEPKSCDKTHTC
PPCPAPELLGGPSVFLFPPKPKDTLMISRTPEVTCVVVDVSHEDPEVKFNWYVDGVE
VHNAKTKPREEQYNSTYRVVSVLTVLHQDWLNGKEYKCKVSNKALPAPIEKTISKA
KGQPREPQVYTLPPSREEMTKNQVSLTCLVKGFYPSDIAVEWESNGQPENNYKTTPP
VLDSDGSFFLYSKLTVDKSRWQQGNVFSCSVMHEALHNHYTQKSLSLSPGK,
and
a light chain having at least 90% (e.g., 90%, 91%, 92%, 93%, 94%, 95%, 96%, 97%,
98%, 99%, or 100%) identity to the sequence of SEQ ID NO: 31:
DIQMTQSPSSLSASVGDRVTITCRASQDISNYINWYQQKPGKAPKLLIYYTSRLYGGV
PSRFSGSGSGTDFTLTISSLQPEDFATYYCQQFSELPRTFGQGTKVEIKRTVAAPSVFIF
PPSDEQLKSGTASVVCLLNNFYPREAKVQWKVDNALQSGNSQESVTEQDSKDSTYS
LSSTLTLSKADYEKHKVYACEVTHQGLSSPVTKSFNRGEC.
In certain embodiments, the antibody comprises a heavy chain having at least 90%
(e.g., 90%, 91%, 92%, 93%, 94%, 95%, 96%, 97%, 98%, 99%, or 100%) identity to the
sequence of SEQ ID NO: 34:
EVQLVESGGGLVQPGGSLRLSCAVSGYIFSSYWVYWVRQAPGKGLEWVGYISPTTG
YTVYADSVKGRFTISRDNSKNTLYLQMNSLRAEDTAVYYCATEGGNWEDYWGQGT
LVTVSSASTKGPSVFPLAPSSKSTSGGTAALGCLVKDYFPEPVTVSWNSGALTSGVH
TFPAVLQSSGLYSLSSVVTVPSSSLGTQTYICNVNHKPSNTKVDKRVEPKSCDKTHTC
PPCPAPEAAGGPSVFLFPPKPKDTLMISRTPEVTCVVVDVSHEDPEVKFNWYVDGVE
VHNAKTKPREEQYNSTYRVVSVLTVLHQDWLNGKEYKCKVSNKALPAPIEKTISKA
KGQPREPQVYTLPPSREEMTKNQVSLTCLVKGFYPSDIAVEWESNGQPENNYKTTPP
VLDSDGSFFLYSKLTVDKSRWQQGNVFSCSVMHEALHNHYTQKSLSLSPGK,
and
a light chain having at least 90% (e.g., 90%, 91%, 92%, 93%, 94%, 95%, 96%, 97%,
98%, 99%, or 100%) identity to the sequence of SEQ ID NO: 31:
DIQMTQSPSSLSASVGDRVTITCRASQDISNYINWYQQKPGKAPKLLIYYTSRLYGGV
PSRFSGSGSGTDFTLTISSLQPEDFATYYCQQFSELPRTFGQGTKVEIKRTVAAPSVFIF
PPSDEQLKSGTASVVCLLNNFYPREAKVQWKVDNALQSGNSQESVTEQDSKDSTYS
LSSTLTLSKADYEKHKVYACEVTHQGLSSPVTKSFNRGEC.
In certain embodiments, the antibody comprises a heavy chain having at least 90%
(e.g., 90%, 91%, 92%, 93%, 94%, 95%, 96%, 97%, 98%, 99%, or 100%) identity to the
sequence of SEQ ID NO: 35:
EVQLVESGGGLVQPGGSLRLSCAVSGYIFSSYWVYWVRQAPGKGLEWVGYISPTTG
YTVYADSVKGRFTISRDNSKNTLYLQMNSLRAEDTAVYYCATEGGNWEDYWGQGT
LVTVSSASTKGPSVFPLAPSSKSTSGGTAALGCLVKDYFPEPVTVSWNSGALTSGVH
TFPAVLQSSGLYSLSSVVTVPSSSLGTQTYICNVNHKPSNTKVDKRVEPKSCDKTHTC
PPCPAPELLGGPSVFLFPPKPKDTLMISRTPEVTCVVVDVSHEDPEVKFNWYVDGVE
VHNAKTKPREEQYASTYRVVSVLTVLHQDWLNGKEYKCKVSNKALPAPIEKTISKA
KGQPREPQVYTLPPSREEMTKNQVSLTCLVKGFYPSDIAVEWESNGQPENNYKTTPP
VLDSDGSFFLYSKLTVDKSRWQQGNVFSCSVMHEALHNHYTQKSLSLSPGK,
and
a light chain having at least 90% (e.g., 90%, 91%, 92%, 93%, 94%, 95%, 96%, 97%,
98%, 99%, or 100%) identity to the sequence of SEQ ID NO: 31:
DIQMTQSPSSLSASVGDRVTITCRASQDISNYINWYQQKPGKAPKLLIYYTSRLYGGV
PSRFSGSGSGTDFTLTISSLQPEDFATYYCQQFSELPRTFGQGTKVEIKRTVAAPSVFIF
PPSDEQLKSGTASVVCLLNNFYPREAKVQWKVDNALQSGNSQESVTEQDSKDSTYS
LSSTLTLSKADYEKHKVYACEVTHQGLSSPVTKSFNRGEC.

### ADWA16hugraft

In particular embodiments, an antibody of the disclosure can comprise: (1) an HCDR1 having the sequence of SEQ ID NO: 5; (2) an HCDR2 having the sequence of SEQ ID NO: 2; (3) an HCDR3 having the sequence of SEQ ID NO: 3; (4) a LCDR1 having the sequence of SEQ ID NO: 14; (5) a LCDR2 having the sequence of SEQ ID NO: 9; and (6) a LCDR3 having the sequence of SEQ ID NO: 10. In some embodiments, the antibody can comprise (1) a heavy chain variable region having an HCDR1, an HCDR2, and an HCDR3 of SEQ ID NOS:5, 2, and 3, respectively, and at least 90% (e.g., 90%, 91%, 92%, 93%, 94%, 95%, 96%, 97%, 98%, 99%, or 100%) identity to the sequence of SEQ ID NO: 17, and (2) a light chain variable region having a LCDR1, a LCDR2, and a LCDR3 of SEQ ID NOS:14, 9, and 10, respectively, and at least 90% (e.g., 90%, 91%, 92%, 93%, 94%, 95%, 96%, 97%, 98%, 99%, or 100%) identity to the sequence of SEQ ID NO: 24. Such an antibody can be an IgG1, IgG2, IgG3, or IgG4.

In certain embodiments, the antibody comprises a heavy chain having at least 90%
(e.g., 90%, 91%, 92%, 93%, 94%, 95%, 96%, 97%, 98%, 99%, or 100%) identity to the
sequence of SEQ ID NO: 36:
EVQLVESGGGLVQPGGSLRLSCAASGYTFSSYWIYWVRQAPGKGLEWVSYINPTTG
YTEYADSVKGRFTISRDNSKNTLYLQMNSLRAEDTAVYYCATEGGNWEDYWGQGT
LVTVSSASTKGPSVFPLAPSSKSTSGGTAALGCLVKDYFPEPVTVSWNSGALTSGVH
TFPAVLQSSGLYSLSSVVTVPSSSLGTQTYICNVNHKPSNTKVDKRVEPKSCDKTHTC
PPCPAPELLGGPSVFLFPPKPKDTLMISRTPEVTCVVVDVSHEDPEVKFNWYVDGVE
VHNAKTKPREEQYNSTYRVVSVLTVLHQDWLNGKEYKCKVSNKALPAPIEKTISKA
KGQPREPQVYTLPPSREEMTKNQVSLTCLVKGFYPSDIAVEWESNGQPENNYKTTPP
VLDSDGSFFLYSKLTVDKSRWQQGNVFSCSVMHEALHNHYTQKSLSLSPGK,
and
a light chain having at least 90% (e.g., 90%, 91%, 92%, 93%, 94%, 95%, 96%, 97%,
98%, 99%, or 100%) identity to the sequence of SEQ ID NO: 37:
DIQMTQSPSSLSASVGDRVTITCRASQDISNYLNWYQQKPGKAPKLLIYYTSRLYSGV
PSRFSGSGSGTDFTLTISSLQPEDFATYYCQQFSELPRTFGQGTKVEIKRTVAAPSVFIF
PPSDEQLKSGTASVVCLLNNFYPREAKVQWKVDNALQSGNSQESVTEQDSKDSTYS
LSSTLTLSKADYEKHKVYACEVTHQGLSSPVTKSFNRGEC.
In certain embodiments, the antibody comprises a heavy chain having at least 90%
(e.g., 90%, 91%, 92%, 93%, 94%, 95%, 96%, 97%, 98%, 99%, or 100%) identity to the
sequence of SEQ ID NO: 38:
EVQLVESGGGLVQPGGSLRLSCAASGYTFSSYWIYWVRQAPGKGLEWVSYINPTTG
YTEYADSVKGRFTISRDNSKNTLYLQMNSLRAEDTAVYYCATEGGNWEDYWGQGT
LVTVSSASTKGPSVFPLAPSSKSTSGGTAALGCLVKDYFPEPVTVSWNSGALTSGVH
TFPAVLQSSGLYSLSSVVTVPSSSLGTQTYICNVNHKPSNTKVDKRVEPKSCDKTHTC
PPCPAPEAAGGPSVFLFPPKPKDTLMISRTPEVTCVVVDVSHEDPEVKFNWYVDGVE
VHNAKTKPREEQYNSTYRVVSVLTVLHQDWLNGKEYKCKVSNKALPAPIEKTISKA
KGQPREPQVYTLPPSREEMTKNQVSLTCLVKGFYPSDIAVEWESNGQPENNYKTTPP
VLDSDGSFFLYSKLTVDKSRWQQGNVFSCSVMHEALHNHYTQKSLSLSPGK,
and
a light chain having at least 90% (e.g., 90%, 91%, 92%, 93%, 94%, 95%, 96%, 97%,
98%, 99%, or 100%) identity to the sequence of SEQ ID NO: 37:
DIQMTQSPSSLSASVGDRVTITCRASQDISNYLNWYQQKPGKAPKLLIYYTSRLYSGV
PSRFSGSGSGTDFTLTISSLQPEDFATYYCQQFSELPRTFGQGTKVEIKRTVAAPSVFIF
PPSDEQLKSGTASVVCLLNNFYPREAKVQWKVDNALQSGNSQESVTEQDSKDSTYS
LSSTLTLSKADYEKHKVYACEVTHQGLSSPVTKSFNRGEC.
In certain embodiments, the antibody comprises a heavy chain having at least 90%
(e.g., 90%, 91%, 92%, 93%, 94%, 95%, 96%, 97%, 98%, 99%, or 100%) identity to the
sequence of SEQ ID NO: 39:
EVQLVESGGGLVQPGGSLRLSCAASGYTFSSYWIYWVRQAPGKGLEWVSYINPTTG
YTEYADSVKGRFTISRDNSKNTLYLQMNSLRAEDTAVYYCATEGGNWEDYWGQGT
LVTVSSASTKGPSVFPLAPSSKSTSGGTAALGCLVKDYFPEPVTVSWNSGALTSGVH
TFPAVLQSSGLYSLSSVVTVPSSSLGTQTYICNVNHKPSNTKVDKRVEPKSCDKTHTC
PPCPAPELLGGPSVFLFPPKPKDTLMISRTPEVTCVVVDVSHEDPEVKFNWYVDGVE
VHNAKTKPREEQYASTYRVVSVLTVLHQDWLNGKEYKCKVSNKALPAPIEKTISKA
KGQPREPQVYTLPPSREEMTKNQVSLTCLVKGFYPSDIAVEWESNGQPENNYKTTPP
VLDSDGSFFLYSKLTVDKSRWQQGNVFSCSVMHEALHNHYTQKSLSLSPGK,
and
a light chain having at least 90% (e.g., 90%, 91%, 92%, 93%, 94%, 95%, 96%, 97%,
98%, 99%, or 100%) identity to the sequence of SEQ ID NO: 37:
DIQMTQSPSSLSASVGDRVTITCRASQDISNYLNWYQQKPGKAPKLLIYYTSRLYSGV
PSRFSGSGSGTDFTLTISSLQPEDFATYYCQQFSELPRTFGQGTKVEIKRTVAAPSVFIF
PPSDEQLKSGTASVVCLLNNFYPREAKVQWKVDNALQSGNSQESVTEQDSKDSTYS
LSSTLTLSKADYEKHKVYACEVTHQGLSSPVTKSFNRGEC.

### ADWA16

In particular embodiments, an antibody of the disclosure can comprise: (1) an HCDR1 having the sequence of SEQ ID NO: 6; (2) an HCDR2 having the sequence of SEQ ID NO: 2; (3) an HCDR3 having the sequence of SEQ ID NO: 3; (4) a LCDR1 having the sequence of SEQ ID NO: 14; (5) a LCDR2 having the sequence of SEQ ID NO: 9; and (6) a LCDR3 having the sequence of SEQ ID NO: 10. In some embodiments, the antibody can comprise (1) a heavy chain variable region having an HCDR1, an HCDR2, and an HCDR3 of SEQ ID NOS:6, 2, and 3, respectively, and at least 90% (e.g., 90%, 91%, 92%, 93%, 94%, 95%, 96%, 97%, 98%, 99%, or 100%) identity to the sequence of SEQ ID NO: 18, and (2) a light chain variable region having a LCDR1, a LCDR2, and a LCDR3 of SEQ ID NOS:14, 9, and 10, respectively, and at least 90% (e.g., 90%, 91%, 92%, 93%, 94%, 95%, 96%, 97%, 98%, 99%, or 100%) identity to the sequence of SEQ ID NO: 25. Such an antibody can be an IgG1, IgG2, IgG3, or IgG4.

In certain embodiments, the antibody comprises a heavy chain having at least 90%
(e.g., 90%, 91%, 92%, 93%, 94%, 95%, 96%, 97%, 98%, 99%, or 100%) identity to the
sequence of SEQ ID NO: 40:
QVQLQQSGAELAKPGASMKMSCKASGYTFSSYWIYWVKQRPGQGLEWIGYINPTT
GYTEYNQKFRDKATLTADKSSNTAYMQLSSLTSEDSAVYYCATEGGNWEDYWGQ
GTTLTVSSASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKRVEPKSCDKTHT
CPPCPAPELLGGPSVFLFPPKPKDTLMISRTPEVTCVVVDVSHEDPEVKFNWYVDGV
EVHNAKTKPREEQYNSTYRVVSVLTVLHQDWLNGKEYKCKVSNKALPAPIEKTISK
AKGQPREPQVYTLPPSREEMTKNQVSLTCLVKGFYPSDIAVEWESNGQPENNYKTTP
PVLDSDGSFFLYSKLTVDKSRWQQGNVFSCSVMHEALHNHYTQKSLSLSPGK,
and
a light chain having at least 90% (e.g., 90%, 91%, 92%, 93%, 94%, 95%, 96%, 97%,
98%, 99%, or 100%) identity to the sequence of SEQ ID NO: 41:
DIQMTQTTSSLSASLGDRVTISCRASQDISNYLNWYQQKPDGTVKLLIYYTSRLYSGV
PSRFSGSGSGTDYSLTISNLEPKDIATYYCQQFSELPRTFGGGTKLEIKRTVAAPSVFIF
PPSDEQLKSGTASVVCLLNNFYPREAKVQWKVDNALQSGNSQESVTEQDSKDSTYS
LSSTLTLSKADYEKHKVYACEVTHQGLSSPVTKSFNRGEC.
In certain embodiments, the antibody comprises a heavy chain having at least 90%
(e.g., 90%, 91%, 92%, 93%, 94%, 95%, 96%, 97%, 98%, 99%, or 100%) identity to the
sequence of SEQ ID NO: 42:
QVQLQQSGAELAKPGASMKMSCKASGYTFSSYWIYWVKQRPGQGLEWIGYINPTT
GYTEYNQKFRDKATLTADKSSNTAYMQLSSLTSEDSAVYYCATEGGNWEDYWGQ
GTTLTVSSASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKRVEPKSCDKTHT
CPPCPAPEAAGGPSVFLFPPKPKDTLMISRTPEVTCVVVDVSHEDPEVKFNWYVDGV
EVHNAKTKPREEQYNSTYRVVSVLTVLHQDWLNGKEYKCKVSNKALPAPIEKTISK
AKGQPREPQVYTLPPSREEMTKNQVSLTCLVKGFYPSDIAVEWESNGQPENNYKTTP
PVLDSDGSFFLYSKLTVDKSRWQQGNVFSCSVMHEALHNHYTQKSLSLSPGK,
and
a light chain having at least 90% (e.g., 90%, 91%, 92%, 93%, 94%, 95%, 96%, 97%,
98%, 99%, or 100%) identity to the sequence of SEQ ID NO: 41:
DIQMTQTTSSLSASLGDRVTISCRASQDISNYLNWYQQKPDGTVKLLIYYTSRLYSGV
PSRFSGSGSGTDYSLTISNLEPKDIATYYCQQFSELPRTFGGGTKLEIKRTVAAPSVFIF
PPSDEQLKSGTASVVCLLNNFYPREAKVQWKVDNALQSGNSQESVTEQDSKDSTYS
LSSTLTLSKADYEKHKVYACEVTHQGLSSPVTKSFNRGEC.
In certain embodiments, the antibody comprises a heavy chain having at least 90%
(e.g., 90%, 91%, 92%, 93%, 94%, 95%, 96%, 97%, 98%, 99%, or 100%) identity to the
sequence of SEQ ID NO: 43:
QVQLQQSGAELAKPGASMKMSCKASGYTFSSYWIYWVKQRPGQGLEWIGYINPTT
GYTEYNQKFRDKATLTADKSSNTAYMQLSSLTSEDSAVYYCATEGGNWEDYWGQ
GTTLTVSSASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKRVEPKSCDKTHT
CPPCPAPELLGGPSVFLFPPKPKDTLMISRTPEVTCVVVDVSHEDPEVKFNWYVDGV
EVHNAKTKPREEQYASTYRVVSVLTVLHQDWLNGKEYKCKVSNKALPAPIEKTISK
AKGQPREPQVYTLPPSREEMTKNQVSLTCLVKGFYPSDIAVEWESNGQPENNYKTTP
PVLDSDGSFFLYSKLTVDKSRWQQGNVFSCSVMHEALHNHYTQKSLSLSPGK,
and
a light chain having at least 90% (e.g., 90%, 91%, 92%, 93%, 94%, 95%, 96%, 97%,
98%, 99%, or 100%) identity to the sequence of SEQ ID NO: 41:
DIQMTQTTSSLSASLGDRVTISCRASQDISNYLNWYQQKPDGTVKLLIYYTSRLYSGV
PSRFSGSGSGTDYSLTISNLEPKDIATYYCQQFSELPRTFGGGTKLEIKRTVAAPSVFIF
PPSDEQLKSGTASVVCLLNNFYPREAKVQWKVDNALQSGNSQESVTEQDSKDSTYS
LSSTLTLSKADYEKHKVYACEVTHQGLSSPVTKSFNRGEC.

### Ab1

In particular embodiments, an antibody of the disclosure can comprise: (1) an HCDR1 having the sequence of SEQ ID NO:1; (2) an HCDR2 having the sequence of SEQ ID NO: 4; (3) an HCDR3 having the sequence of SEQ ID NO: 3; (4) a LCDR1 having the sequence of SEQ ID NO: 8; (5) a LCDR2 having the sequence of SEQ ID NO: 9; and (6) a LCDR3 having the sequence of SEQ ID NO: 10. In some embodiments, the antibody can comprise (1) a heavy chain variable region having an HCDR1, an HCDR2, and an HCDR3 of SEQ ID NOS:1, 4, and 3, respectively, and at least 90% (e.g., 90%, 91%, 92%, 93%, 94%, 95%, 96%, 97%, 98%, 99%, or 100%) identity to the sequence of SEQ ID NO: 16, and (2) a light chain variable region having a LCDR1, a LCDR2, and a LCDR3 of SEQ ID NOS:8-10, respectively, and at least 90% (e.g., 90%, 91%, 92%, 93%, 94%, 95%, 96%, 97%, 98%, 99%, or 100%) identity to the sequence of SEQ ID NO: 20. Such an antibody can be an IgG1, IgG2, IgG3, or IgG4.

In certain embodiments, the antibody comprises a heavy chain having at least 90%
(e.g., 90%, 91%, 92%, 93%, 94%, 95%, 96%, 97%, 98%, 99%, or 100%) identity to the
sequence of SEQ ID NO: 33:
EVQLVESGGGLVQPGGSLRLSCAVSGYIFSSYWVYWVRQAPGKGLEWVGYISPTTG
YTVYADSVKGRFTISRDNSKNTLYLQMNSLRAEDTAVYYCATEGGNWEDYWGQGT
LVTVSSASTKGPSVFPLAPSSKSTSGGTAALGCLVKDYFPEPVTVSWNSGALTSGVH
TFPAVLQSSGLYSLSSVVTVPSSSLGTQTYICNVNHKPSNTKVDKRVEPKSCDKTHTC
PPCPAPELLGGPSVFLFPPKPKDTLMISRTPEVTCVVVDVSHEDPEVKFNWYVDGVE
VHNAKTKPREEQYNSTYRVVSVLTVLHQDWLNGKEYKCKVSNKALPAPIEKTISKA
KGQPREPQVYTLPPSREEMTKNQVSLTCLVKGFYPSDIAVEWESNGQPENNYKTTPP
VLDSDGSFFLYSKLTVDKSRWQQGNVFSCSVMHEALHNHYTQKSLSLSPGK,
and
a light chain having at least 90% (e.g., 90%, 91%, 92%, 93%, 94%, 95%, 96%, 97%,
98%, 99%, or 100%) identity to the sequence of SEQ ID NO: 27:
DIQMTQSPSSLSASVGDRVTITCRASQDISNYINWYQQKPGKAPKLLIYYTSRLYSGV
PSRFSGSGSGTDFTLTISSLQPEDFATYYCQQFSELPRTFGQGTKVEIKRTVAAPSVFIF
PPSDEQLKSGTASVVCLLNNFYPREAKVQWKVDNALQSGNSQESVTEQDSKDSTYS
LSSTLTLSKADYEKHKVYACEVTHQGLSSPVTKSFNRGEC.
In certain embodiments, the antibody comprises a heavy chain having at least 90%
(e.g., 90%, 91%, 92%, 93%, 94%, 95%, 96%, 97%, 98%, 99%, or 100%) identity to the
sequence of SEQ ID NO: 34:
EVQLVESGGGLVQPGGSLRLSCAVSGYIFSSYWVYWVRQAPGKGLEWVGYISPTTG
YTVYADSVKGRFTISRDNSKNTLYLQMNSLRAEDTAVYYCATEGGNWEDYWGQGT
LVTVSSASTKGPSVFPLAPSSKSTSGGTAALGCLVKDYFPEPVTVSWNSGALTSGVH
TFPAVLQSSGLYSLSSVVTVPSSSLGTQTYICNVNHKPSNTKVDKRVEPKSCDKTHTC
PPCPAPEAAGGPSVFLFPPKPKDTLMISRTPEVTCVVVDVSHEDPEVKFNWYVDGVE
VHNAKTKPREEQYNSTYRVVSVLTVLHQDWLNGKEYKCKVSNKALPAPIEKTISKA
KGQPREPQVYTLPPSREEMTKNQVSLTCLVKGFYPSDIAVEWESNGQPENNYKTTPP
VLDSDGSFFLYSKLTVDKSRWQQGNVFSCSVMHEALHNHYTQKSLSLSPGK,
and
a light chain having at least 90% (e.g., 90%, 91%, 92%, 93%, 94%, 95%, 96%, 97%,
98%, 99%, or 100%) identity to the sequence of SEQ ID NO: 27:
DIQMTQSPSSLSASVGDRVTITCRASQDISNYINWYQQKPGKAPKLLIYYTSRLYSGV
PSRFSGSGSGTDFTLTISSLQPEDFATYYCQQFSELPRTFGQGTKVEIKRTVAAPSVFIF
PPSDEQLKSGTASVVCLLNNFYPREAKVQWKVDNALQSGNSQESVTEQDSKDSTYS
LSSTLTLSKADYEKHKVYACEVTHQGLSSPVTKSFNRGEC.
In certain embodiments, the antibody comprises a heavy chain having at least 90%
(e.g., 90%, 91%, 92%, 93%, 94%, 95%, 96%, 97%, 98%, 99%, or 100%) identity to the
sequence of SEQ ID NO: 35:
EVQLVESGGGLVQPGGSLRLSCAVSGYIFSSYWVYWVRQAPGKGLEWVGYISPTTG
YTVYADSVKGRFTISRDNSKNTLYLQMNSLRAEDTAVYYCATEGGNWEDYWGQGT
LVTVSSASTKGPSVFPLAPSSKSTSGGTAALGCLVKDYFPEPVTVSWNSGALTSGVH
TFPAVLQSSGLYSLSSVVTVPSSSLGTQTYICNVNHKPSNTKVDKRVEPKSCDKTHTC
PPCPAPELLGGPSVFLFPPKPKDTLMISRTPEVTCVVVDVSHEDPEVKFNWYVDGVE
VHNAKTKPREEQYASTYRVVSVLTVLHQDWLNGKEYKCKVSNKALPAPIEKTISKA
KGQPREPQVYTLPPSREEMTKNQVSLTCLVKGFYPSDIAVEWESNGQPENNYKTTPP
VLDSDGSFFLYSKLTVDKSRWQQGNVFSCSVMHEALHNHYTQKSLSLSPGK,
and
a light chain having at least 90% (e.g., 90%, 91%, 92%, 93%, 94%, 95%, 96%, 97%,
98%, 99%, or 100%) identity to the sequence of SEQ ID NO: 27:
DIQMTQSPSSLSASVGDRVTITCRASQDISNYINWYQQKPGKAPKLLIYYTSRLYSGV
PSRFSGSGSGTDFTLTISSLQPEDFATYYCQQFSELPRTFGQGTKVEIKRTVAAPSVFIF
PPSDEQLKSGTASVVCLLNNFYPREAKVQWKVDNALQSGNSQESVTEQDSKDSTYS
LSSTLTLSKADYEKHKVYACEVTHQGLSSPVTKSFNRGEC.

### Ab2

In particular embodiments, an antibody of the disclosure can comprise: (1) an HCDR1 having the sequence of SEQ ID NO:1; (2) an HCDR2 having the sequence of SEQ ID NO: 7; (3) an HCDR3 having the sequence of SEQ ID NO: 3; (4) a LCDR1 having the sequence of SEQ ID NO: 8; (5) a LCDR2 having the sequence of SEQ ID NO: 12; and (6) a LCDR3 having the sequence of SEQ ID NO: 10. In some embodiments, the antibody can comprise (1) a heavy chain variable region having an HCDR1, an HCDR2, and an HCDR3 of SEQ ID NOS:1, 7, and 3, respectively, and at least 90% (e.g., 90%, 91%, 92%, 93%, 94%, 95%, 96%, 97%, 98%, 99%, or 100%) identity to the sequence of SEQ ID NO: 19, and (2) a light chain variable region having a LCDR1, a LCDR2, and a LCDR3 of SEQ ID NOS:8, 12, and 10, respectively, and at least 90% (e.g., 90%, 91%, 92%, 93%, 94%, 95%, 96%, 97%, 98%, 99%, or 100%) identity to the sequence of SEQ ID NO: 22. Such an antibody can be an IgG1, IgG2, IgG3, or IgG4.

In certain embodiments, the antibody comprises a heavy chain having at least 90%
(e.g., 90%, 91%, 92%, 93%, 94%, 95%, 96%, 97%, 98%, 99%, or 100%) identity to the
sequence of SEQ ID NO: 44:
EVQLVESGGGLVQPGGSLRLSCAVSGYIFSSYWVYWVRQAPGKGLEWVGYISPTTG
YTEYADSVKGRFTISRDNSKNTLYLQMNSLRAEDTAVYYCATEGGNWEDYWGQGT
LVTVSSASTKGPSVFPLAPSSKSTSGGTAALGCLVKDYFPEPVTVSWNSGALTSGVH
TFPAVLQSSGLYSLSSVVTVPSSSLGTQTYICNVNHKPSNTKVDKRVEPKSCDKTHTC
PPCPAPELLGGPSVFLFPPKPKDTLMISRTPEVTCVVVDVSHEDPEVKFNWYVDGVE
VHNAKTKPREEQYNSTYRVVSVLTVLHQDWLNGKEYKCKVSNKALPAPIEKTISKA
KGQPREPQVYTLPPSREEMTKNQVSLTCLVKGFYPSDIAVEWESNGQPENNYKTTPP
VLDSDGSFFLYSKLTVDKSRWQQGNVFSCSVMHEALHNHYTQKSLSLSPGK,
and
a light chain having at least 90% (e.g., 90%, 91%, 92%, 93%, 94%, 95%, 96%, 97%,
98%, 99%, or 100%) identity to the sequence of SEQ ID NO: 31:
DIQMTQSPSSLSASVGDRVTITCRASQDISNYINWYQQKPGKAPKLLIYYTSRLYGGV
PSRFSGSGSGTDFTLTISSLQPEDFATYYCQQFSELPRTFGQGTKVEIKRTVAAPSVFIF
PPSDEQLKSGTASVVCLLNNFYPREAKVQWKVDNALQSGNSQESVTEQDSKDSTYS
LSSTLTLSKADYEKHKVYACEVTHQGLSSPVTKSFNRGEC.
In certain embodiments, the antibody comprises a heavy chain having at least 90%
(e.g., 90%, 91%, 92%, 93%, 94%, 95%, 96%, 97%, 98%, 99%, or 100%) identity to the
sequence of SEQ ID NO: 45:
EVQLVESGGGLVQPGGSLRLSCAVSGYIFSSYWVYWVRQAPGKGLEWVGYISPTTG
YTEYADSVKGRFTISRDNSKNTLYLQMNSLRAEDTAVYYCATEGGNWEDYWGQGT
LVTVSSASTKGPSVFPLAPSSKSTSGGTAALGCLVKDYFPEPVTVSWNSGALTSGVH
TFPAVLQSSGLYSLSSVVTVPSSSLGTQTYICNVNHKPSNTKVDKRVEPKSCDKTHTC
PPCPAPEAAGGPSVFLFPPKPKDTLMISRTPEVTCVVVDVSHEDPEVKFNWYVDGVE
VHNAKTKPREEQYNSTYRVVSVLTVLHQDWLNGKEYKCKVSNKALPAPIEKTISKA
KGQPREPQVYTLPPSREEMTKNQVSLTCLVKGFYPSDIAVEWESNGQPENNYKTTPP
VLDSDGSFFLYSKLTVDKSRWQQGNVFSCSVMHEALHNHYTQKSLSLSPGK,
and
a light chain having at least 90% (e.g., 90%, 91%, 92%, 93%, 94%, 95%, 96%, 97%,
98%, 99%, or 100%) identity to the sequence of SEQ ID NO: 31:
DIQMTQSPSSLSASVGDRVTITCRASQDISNYINWYQQKPGKAPKLLIYYTSRLYGGV
PSRFSGSGSGTDFTLTISSLQPEDFATYYCQQFSELPRTFGQGTKVEIKRTVAAPSVFIF
PPSDEQLKSGTASVVCLLNNFYPREAKVQWKVDNALQSGNSQESVTEQDSKDSTYS
LSSTLTLSKADYEKHKVYACEVTHQGLSSPVTKSFNRGEC
In certain embodiments, the antibody comprises a heavy chain having at least 90%
(e.g., 90%, 91%, 92%, 93%, 94%, 95%, 96%, 97%, 98%, 99%, or 100%) identity to the
sequence of SEQ ID NO: 46:
EVQLVESGGGLVQPGGSLRLSCAVSGYIFSSYWVYWVRQAPGKGLEWVGYISPTTG
YTEYADSVKGRFTISRDNSKNTLYLQMNSLRAEDTAVYYCATEGGNWEDYWGQGT
LVTVSSASTKGPSVFPLAPSSKSTSGGTAALGCLVKDYFPEPVTVSWNSGALTSGVH
TFPAVLQSSGLYSLSSVVTVPSSSLGTQTYICNVNHKPSNTKVDKRVEPKSCDKTHTC
PPCPAPELLGGPSVFLFPPKPKDTLMISRTPEVTCVVVDVSHEDPEVKFNWYVDGVE
VHNAKTKPREEQYASTYRVVSVLTVLHQDWLNGKEYKCKVSNKALPAPIEKTISKA
KGQPREPQVYTLPPSREEMTKNQVSLTCLVKGFYPSDIAVEWESNGQPENNYKTTPP
VLDSDGSFFLYSKLTVDKSRWQQGNVFSCSVMHEALHNHYTQKSLSLSPGK,
and
a light chain having at least 90% (e.g., 90%, 91%, 92%, 93%, 94%, 95%, 96%, 97%,
98%, 99%, or 100%) identity to the sequence of SEQ ID NO: 31:
DIQMTQSPSSLSASVGDRVTITCRASQDISNYINWYQQKPGKAPKLLIYYTSRLYGGV
PSRFSGSGSGTDFTLTISSLQPEDFATYYCQQFSELPRTFGQGTKVEIKRTVAAPSVFIF
PPSDEQLKSGTASVVCLLNNFYPREAKVQWKVDNALQSGNSQESVTEQDSKDSTYS
LSSTLTLSKADYEKHKVYACEVTHQGLSSPVTKSFNRGEC.

### Ab3

In particular embodiments, an antibody of the disclosure can comprise: (1) an HCDR1 having the sequence of SEQ ID NO:1; (2) an HCDR2 having the sequence of SEQ ID NO: 7; (3) an HCDR3 having the sequence of SEQ ID NO: 3; (4) a LCDR1 having the sequence of SEQ ID NO: 8; (5) a LCDR2 having the sequence of SEQ ID NO: 9; and (6) a LCDR3 having the sequence of SEQ ID NO: 10. In some embodiments, the antibody can comprise (1) a heavy chain variable region having an HCDR1, an HCDR2, and an HCDR3 of SEQ ID NOS:1, 7, and 3, respectively, and at least 90% (e.g., 90%, 91%, 92%, 93%, 94%, 95%, 96%, 97%, 98%, 99%, or 100%) identity to the sequence of SEQ ID NO: 19, and (2) a light chain variable region having a LCDR1, a LCDR2, and a LCDR3 of SEQ ID NOS:8-10, respectively, and at least 90% (e.g., 90%, 91%, 92%, 93%, 94%, 95%, 96%, 97%, 98%, 99%, or 100%) identity to the sequence of SEQ ID NO: 20. Such an antibody can be an IgG1, IgG2, IgG3, or IgG4.

In certain embodiments, the antibody comprises a heavy chain having at least 90%
(e.g., 90%, 91%, 92%, 93%, 94%, 95%, 96%, 97%, 98%, 99%, or 100%) identity to the
sequence of SEQ ID NO: 44:
EVQLVESGGGLVQPGGSLRLSCAVSGYIFSSYWVYWVRQAPGKGLEWVGYISPTTG
YTEYADSVKGRFTISRDNSKNTLYLQMNSLRAEDTAVYYCATEGGNWEDYWGQGT
LVTVSSASTKGPSVFPLAPSSKSTSGGTAALGCLVKDYFPEPVTVSWNSGALTSGVH
TFPAVLQSSGLYSLSSVVTVPSSSLGTQTYICNVNHKPSNTKVDKRVEPKSCDKTHTC
PPCPAPELLGGPSVFLFPPKPKDTLMISRTPEVTCVVVDVSHEDPEVKFNWYVDGVE
VHNAKTKPREEQYNSTYRVVSVLTVLHQDWLNGKEYKCKVSNKALPAPIEKTISKA
KGQPREPQVYTLPPSREEMTKNQVSLTCLVKGFYPSDIAVEWESNGQPENNYKTTPP
VLDSDGSFFLYSKLTVDKSRWQQGNVFSCSVMHEALHNHYTQKSLSLSPGK,
and
a light chain having at least 90% (e.g., 90%, 91%, 92%, 93%, 94%, 95%, 96%, 97%,
98%, 99%, or 100%) identity to the sequence of SEQ ID NO: 27:
DIQMTQSPSSLSASVGDRVTITCRASQDISNYINWYQQKPGKAPKLLIYYTSRLYSGV
PSRFSGSGSGTDFTLTISSLQPEDFATYYCQQFSELPRTFGQGTKVEIKRTVAAPSVFIF
PPSDEQLKSGTASVVCLLNNFYPREAKVQWKVDNALQSGNSQESVTEQDSKDSTYS
LSSTLTLSKADYEKHKVYACEVTHQGLSSPVTKSFNRGEC.
In certain embodiments, the antibody comprises a heavy chain having at least 90%
(e.g., 90%, 91%, 92%, 93%, 94%, 95%, 96%, 97%, 98%, 99%, or 100%) identity to the
sequence of SEQ ID NO: 45:
EVQLVESGGGLVQPGGSLRLSCAVSGYIFSSYWVYWVRQAPGKGLEWVGYISPTTG
YTEYADSVKGRFTISRDNSKNTLYLQMNSLRAEDTAVYYCATEGGNWEDYWGQGT
LVTVSSASTKGPSVFPLAPSSKSTSGGTAALGCLVKDYFPEPVTVSWNSGALTSGVH
TFPAVLQSSGLYSLSSVVTVPSSSLGTQTYICNVNHKPSNTKVDKRVEPKSCDKTHTC
PPCPAPEAAGGPSVFLFPPKPKDTLMISRTPEVTCVVVDVSHEDPEVKFNWYVDGVE
VHNAKTKPREEQYNSTYRVVSVLTVLHQDWLNGKEYKCKVSNKALPAPIEKTISKA
KGQPREPQVYTLPPSREEMTKNQVSLTCLVKGFYPSDIAVEWESNGQPENNYKTTPP
VLDSDGSFFLYSKLTVDKSRWQQGNVFSCSVMHEALHNHYTQKSLSLSPGK,
and
a light chain having at least 90% (e.g., 90%, 91%, 92%, 93%, 94%, 95%, 96%, 97%,
98%, 99%, or 100%) identity to the sequence of SEQ ID NO: 27:
DIQMTQSPSSLSASVGDRVTITCRASQDISNYINWYQQKPGKAPKLLIYYTSRLYSGV
PSRFSGSGSGTDFTLTISSLQPEDFATYYCQQFSELPRTFGQGTKVEIKRTVAAPSVFIF
PPSDEQLKSGTASVVCLLNNFYPREAKVQWKVDNALQSGNSQESVTEQDSKDSTYS
LSSTLTLSKADYEKHKVYACEVTHQGLSSPVTKSFNRGEC.
In certain embodiments, the antibody comprises a heavy chain having at least 90%
(e.g., 90%, 91%, 92%, 93%, 94%, 95%, 96%, 97%, 98%, 99%, or 100%) identity to the
sequence of SEQ ID NO: 46:
EVQLVESGGGLVQPGGSLRLSCAVSGYIFSSYWVYWVRQAPGKGLEWVGYISPTTG
YTEYADSVKGRFTISRDNSKNTLYLQMNSLRAEDTAVYYCATEGGNWEDYWGQGT
LVTVSSASTKGPSVFPLAPSSKSTSGGTAALGCLVKDYFPEPVTVSWNSGALTSGVH
TFPAVLQSSGLYSLSSVVTVPSSSLGTQTYICNVNHKPSNTKVDKRVEPKSCDKTHTC
PPCPAPELLGGPSVFLFPPKPKDTLMISRTPEVTCVVVDVSHEDPEVKFNWYVDGVE
VHNAKTKPREEQYASTYRVVSVLTVLHQDWLNGKEYKCKVSNKALPAPIEKTISKA
KGQPREPQVYTLPPSREEMTKNQVSLTCLVKGFYPSDIAVEWESNGQPENNYKTTPP
VLDSDGSFFLYSKLTVDKSRWQQGNVFSCSVMHEALHNHYTQKSLSLSPGK,
and
a light chain having at least 90% (e.g., 90%, 91%, 92%, 93%, 94%, 95%, 96%, 97%,
98%, 99%, or 100%) identity to the sequence of SEQ ID NO: 27:
DIQMTQSPSSLSASVGDRVTITCRASQDISNYINWYQQKPGKAPKLLIYYTSRLYSGV
PSRFSGSGSGTDFTLTISSLQPEDFATYYCQQFSELPRTFGQGTKVEIKRTVAAPSVFIF
PPSDEQLKSGTASVVCLLNNFYPREAKVQWKVDNALQSGNSQESVTEQDSKDSTYS
LSSTLTLSKADYEKHKVYACEVTHQGLSSPVTKSFNRGEC.

In some embodiments, the CDR1, CDR2, and CDR3 of the heavy chain variable region and the CDR1, CDR2, and CDR3 of the light chain variable region are determined by the North method (see, e.g., North et al., J Mol Biol. 406(2):228-256, 2011). In some embodiments, the antibody comprises the CDR1, CDR2, and CDR3, as determined by the North method (see, e.g., North et al., J Mol Biol. 406(2):228-256, 2011), of the heavy and light chain variable regions of an antibody selected from ADWA16-1, ADWA16-2, ADWA16-3, ADWA16-4, ADWA16-3.2, ADWA16hugraft, ADWA16, Ab1, Ab2, and Ab3.

In other embodiments, the CDRs of an antibody can be determined by Kabat numbering scheme from the heavy chain variable regions and light chain variable regions provided herein.

Any of the antibodies described herein can include one or more human framework region (e.g., 1, 2, 3, or 4 FRs). In some embodiments, the one or more human framework region includes at least one back mutation.

In further embodiments, an antibody described herein can cross-react with mouse integrin β8. In certain embodiments, the antibody can block TGFβ activation. Moreover, the antibody can antagonize binding of LAP to αvβ8 with an IC₅₀ below 5 nM (e.g., below 4.5 nM, 4 nM, 3.5 nM, 3 nM, 2.5 nM, 2 nM, 1.5 nM, 1 nM, or 0.5 nM).

In some embodiments, a modification can optionally be introduced into the antibodies (e.g., within the polypeptide chain or at either the N- or C-terminal), e.g., to extend in vivo half-life, such as PEGylation or incorporation of long-chain polyethylene glycol polymers (PEG). Introduction of PEG or long chain polymers of PEG increases the effective molecular weight of the polypeptides, for example, to prevent rapid filtration into the urine. In some embodiments, a Lysine residue in the sequence is conjugated to PEG directly or through a linker. Such linker can be, for example, a Glu residue or an acyl residue containing a thiol functional group for linkage to the appropriately modified PEG chain. An alternative method for introducing a PEG chain is to first introduce a Cys residue at the C-terminus or at solvent exposed residues such as replacements for Arg or Lys residues. This Cys residue is then site-specifically attached to a PEG chain containing, for example, a maleimide function. Methods for incorporating PEG or long chain polymers of PEG are known in the art (described, for example, in Veronese, F. M., et al., Drug Disc. Today 10: 1451-8 (2005); Greenwald, R. B., et al., Adv. Drug Deliv. Rev. 55: 217-50 (2003); Roberts, M. J., et al., Adv. Drug Deliv. Rev., 54: 459-76 (2002)), the contents of which are incorporated herein by reference.

In certain embodiments, specific mutations of antibodies can be made to alter the glycosylation of the polypeptide. Such mutations may be selected to introduce or eliminate one or more glycosylation sites, including but not limited to, O-linked or N-linked glycosylation sites. In certain embodiments, the proteins have glycosylation sites and patterns unaltered relative to the naturally-occurring proteins. In certain embodiments, a variant of proteins includes a glycosylation variant wherein the number and/or type of glycosylation sites have been altered relative to the naturally-occurring proteins. In certain embodiments, a variant of a polypeptide comprises a greater or a lesser number of N-linked glycosylation sites relative to a native polypeptide. An N-linked glycosylation site is characterized by the sequence: Asn-X-Ser or Asn-X-Thr, wherein the amino acid residue designated as X may be any amino acid residue. The substitution of amino acid residues to create this sequence provides a potential new site for the addition of an N-linked carbohydrate chain. Alternatively, substitutions which eliminate this sequence will remove an existing N-linked carbohydrate chain. In certain embodiments, a rearrangement of N-linked carbohydrate chains is provided, wherein one or more N-linked glycosylation sites (typically those that are naturally occurring) are eliminated and one or more new N-linked sites are created. In some embodiments, antibodies described herein have an amino acid substitution introduced in the HCDR2 sequence to eliminate an N-linked glycosylation site. In some embodiments, the N-glycosylation site, as shown in bold in the sequence of YINPTTGYTE (SEQ ID NO:2), can undergo amino acid substitution from N to S, N to I, or N to V.

Monoclonal antibodies, and chimeric, and especially humanized antibodies, are of particular use for human therapeutic uses of the antibodies described herein. Monoclonal antibodies can be obtained by various techniques familiar to those skilled in the art. Briefly, spleen cells from an animal immunized with a desired antigen are immortalized, commonly by fusion with a myeloma cell (see, for example, Kohler & Milstein, Eur. J. Immunol. 6: 511-519 (1976)). Alternative methods of immortalization include transformation with Epstein Barr Virus, oncogenes, or retroviruses, or other methods well known in the art. Colonies arising from single immortalized cells are screened for production of antibodies of the desired specificity and affinity for the antigen, and yield of the monoclonal antibodies produced by such cells may be enhanced by various techniques, including injection into the peritoneal cavity of a vertebrate host. Alternatively, one may isolate DNA sequences which encode a monoclonal antibody or a binding fragment thereof by screening a DNA library from human B cells according to the general protocol outlined by Huse et al., Science 246: 1275-1281 (1989).

Further, monoclonal antibodies can be collected and titered against a β8 ligand (e.g., LAP) in an immunoassay, for example, a solid phase immunoassay with the ligand immobilized on a solid support. In some embodiments, monoclonal antibodies can bind with a K_{d} of at least about 0.1 mM, e.g., at least about 1 μM, e.g., at least about 0.1 μM or better, e.g., 0.01 μM or lower.

In an exemplary embodiment, an animal, such as a rabbit or mouse can be immunized with a β8 polypeptide, or an nucleic acid construct encoding such a polypeptide. The antibodies produced as a result of the immunization can be isolated using standard methods. In some embodiments, the animal is a knockout of integrin β8 and is immunized with a human β8 integrin polypeptide or a fragment thereof.

The immunoglobulins, including binding fragments and other derivatives thereof, of the present disclosure may be produced readily by a variety of recombinant DNA techniques, including by expression in transfected cells (e.g., immortalized eukaryotic cells, such as myeloma or hybridoma cells) or in mice, rats, rabbits, or other vertebrate capable of producing antibodies by well-known methods. Suitable source cells for the DNA sequences and host cells for immunoglobulin expression and secretion can be obtained from a number of sources, such as the American Type Culture Collection (Catalogue of Cell Lines and Hybridomas, Fifth edition (1985) Rockville, Md).

In some embodiments, the antibody is a humanized antibody, i.e., an antibody that retains the reactivity of a non-human antibody while being less immunogenic in humans. This can be achieved, for instance, by retaining the non-human CDR regions and replacing the remaining parts of the antibody with their human counterparts. See, e.g., Morrison et al., PNAS USA, 81:6851-6855 (1984); Morrison and Oi, Adv. Immunol., 44:65-92 (1988); Verhoeyen et al., Science, 239:1534-1536 (1988); Padlan, Molec. Immun., 28:489-498 (1991); Padlan, Molec. Immun., 31(3):169-217 (1994). Techniques for humanizing antibodies are well known in the art and are described in e.g., U.S. Pat. Nos. 4,816,567; 5,530,101; 5,859,205; 5,585,089; 5,693,761; 5,693,762; 5,777,085; 6,180,370; 6,210,671; and 6,329,511; WO 87/02671; EP Patent Application 0173494; Jones et al. (1986) Nature 321:522; and Verhoyen et al. (1988) Science 239:1534. Humanized antibodies are further described in, e.g., Winter and Milstein (1991) Nature 349:293. For example, polynucleotides comprising a first sequence coding for humanized immunoglobulin framework regions and a second sequence set coding for the desired immunoglobulin complementarity determining regions can be produced synthetically or by combining appropriate cDNA and genomic DNA segments. Human constant region DNA sequences can be isolated in accordance with well-known procedures from a variety of human cells. The CDRs for producing the immunoglobulins of the present disclosure can be similarly derived from monoclonal antibodies capable of specifically binding to αvβ8 integrin.

In some embodiments, the antibodies are antibody fragments such as Fab, F(ab′)₂, Fv or scFv. The antibody fragments can be generated using any means known in the art including, chemical digestion (e.g., papain or pepsin) and recombinant methods. Methods for isolating and preparing recombinant nucleic acids are known to those skilled in the art (see, Sambrook et al., Molecular Cloning. A Laboratory Manual (2d ed. 1989); Ausubel et al., Current Protocols in Molecular Biology (1995)). The antibodies can be expressed in a variety of host cells, including E. coli, other bacterial hosts, yeast, and various higher eukaryotic cells such as the COS, CHO, and HeLa cells lines and myeloma cell lines.

Competitive binding assays can be used to identify antibodies that compete with an antibody described herein for specific binding to αvβ8 integrin. Any of a number of competitive binding assays known in the art can be used to measure competition between two antibodies to the same antigen. Briefly, the ability of different antibodies to inhibit the binding of another antibody can be tested. For example, antibodies can be differentiated by the epitope to which they bind using a sandwich ELISA assay. This can be carried out by using a capture antibody to coat the surface of a well. A subsaturating concentration of tagged-antigen can then be added to the capture surface. This protein can be bound to the antibody through a specific antibody:epitope interaction. After washing, a second antibody, which has been covalently linked to a detectable moiety (e.g., HRP, with the labeled antibody being defined as the detection antibody) can be added to the ELISA. If this antibody recognizes the same epitope as the capture antibody it would be unable to bind to the target protein as that particular epitope would no longer be available for binding. If however this second antibody recognizes a different epitope on the target protein it would be able to bind and this binding can be detected by quantifying the level of activity (and hence antibody bound) using a relevant substrate. The background can be defined by using a single antibody as both capture and detection antibody, whereas the maximal signal can be established by capturing with an antigen specific antibody and detecting with an antibody to the tag on the antigen. By using the background and maximal signals as references, antibodies can be assessed in a pair-wise manner to determine epitope specificity. In some embodiments, a first antibody is considered to competitively inhibit binding of a second antibody, if binding of the second antibody to the antigen is reduced by at least 30%, usually at least about 40%, 50%, 60% or 75%, and often by at least about 90%, in the presence of the first antibody using any of the assays described above.

### IV. Fc Polypeptide

An antibody described herein can comprise an Fc polypeptide. The Fc polypeptide can be a wild-type Fc polypeptide, e.g., a human IgG1 Fc polypeptide. In certain embodiments, an antibody described herein can comprise a wild-type Fc polypeptide having the sequence of SEQ ID NO:47:

APELLGGPSVFLFPPKPKDTLMISRTPEVTCVVVDVSHEDPEVKFNWYVDGVEVHNA KTKPREEQYNSTYRVVSVLTVLHQDWLNGKEYKCKVSNKALPAPIEKTISKAKGQP REPQVYTLPPSREEMTKNQVSLTCLVKGFYPSDIAVEWESNGQPENNYKTTPPVLDS DGSFFLYSKLTVDKSRWQQGNVFSCSVMHEALHNHYTQKSLSLSPGK. In other embodiments, an antibody described herein can comprise a variant of the wild-type Fc polypeptide that has at least 90% (e.g., 90%, 91%, 92%, 93%, 94%, 95%, 96%, 97%, 98%, 99%, or 99.5%) identity to the sequence of a wild-type Fc polypeptide (e.g., SEQ ID NO:47) and at least one amino acid substitution relative to the sequence of a wild-type Fc polypeptide (e.g., SEQ ID NO:47).

In some embodiments, an Fc polypeptide in an antibody described herein can include amino acid substitutions that modulate effector function. In certain embodiments, an Fc polypeptide in an antibody described herein can include amino acid substitutions that reduce or eliminate effector function. Illustrative Fc polypeptide amino acid substitutions that reduce effector function include, but are not limited to, substitutions in a CH2 domain, e.g., at positions 234 and 235 (position numbering relative to the sequence of SEQ ID NO:26) or at positions 4 and 5 (position numbering relative to the sequence of SEQ ID NO:47) (see, e.g., Lund et al., J Immunol. 147(8):2657-62, 1991). For example, in some embodiments, one or both Fc polypeptides in an antibody described herein can comprise L234A and L235A substitutions. In particular embodiments, one or both Fc polypeptides in an antibody described herein can have the sequence of SEQ ID NO:48:

APEAAGGPSVFLFPPKPKDTLMISRTPEVTCVVVDVSHEDPEVKFNWYV
DGVEVHNAKTKPREEQYNSTYRVVSVLTVLHQDWLNGKEYKCKVSNKAL
PAPIEKTISKAKGQPREPQVYTLPPSREEMTKNQVSLTCLVKGFYPSDI
AVEWESNGQPENNYKTTPPVLDSDGSFFLYSKLTVDKSRWQQGNVFSCS
VMHEALHNHYTQKSLSLSPGK.

Additional Fc polypeptide amino acid substitutions that modulate an effector function include, e.g., substitution at position 329 and substitution at position 297 (position numbering relative to the sequence of SEQ ID NO:26). For example, in some embodiments, one or both Fc polypeptides in an antibody described herein can comprise P329G substitution. In certain embodiments, one or both Fc polypeptides in an antibody described herein can have L234A, L235A, and P329G substitutions. In particular embodiments, one or both Fc polypeptides in an antibody described herein can have the sequence of SEQ ID NO:49:

APEAAGGPSVFLFPPKPKDTLMISRTPEVTCVVVDVSHEDPEVKFNWYV
DGVEVHNAKTKPREEQYNSTYRVVSVLTVLHQDWLNGKEYKCKVSNKAL
GAPIEKTISKAKGQPREPQVYTLPPSREEMTKNQVSLTCLVKGFYPSDI
AVEWESNGQPENNYKTTPPVLDSDGSFFLYSKLTVDKSRWQQGNVFSCS
VMHEALHNHYTQKSLSLSPGK.

Furthermore, one or both Fc polypeptides in an antibody described herein can comprise N297A substitution (position numbering relative to the sequence of SEQ ID NO:26) or N67A substitution (position numbering relative to the sequence of SEQ ID NO:47) (see, e.g., Tao and Morrison, J Immunol. 143(8):2595-601, 1989). In particular embodiments, one or both Fc polypeptides in an antibody described herein can have the sequence of SEQ ID NO:50.

APELLGGPSVFLFPPKPKDTLMISRTPEVTCVVVDVSHEDPEVKFNWYV
DGVEVHNAKTKPREEQYASTYRVVSVLTVLHQDWLNGKEYKCKVSNKAL
PAPIEKTISKAKGQPREPQVYTLPPSREEMTKNQVSLTCLVKGFYPSDI
AVEWESNGQPENNYKTTPPVLDSDGSFFLYSKLTVDKSRWQQGNVFSCS
VMHEALHNHYTQKSLSLSPGK

### V. Therapeutic Treatment

The antibodies (including antibody fragments) described herein can be used to reduce TGFβ activation in a cell or an animal. Accordingly, the antibodies can be administered to an animal (e.g., a human or non-human animal) in need thereof, thereby reducing TGFβ activation in the animal. Diseases for which reduction of TGFβ is at least ameliorative include, but are not limited to, asthma, multiple sclerosis, acute lung injury, rheumatoid arthritis, psoriasis and chronic obstructive pulmonary disease. For example, the inventors have found that β8 knockout mice have ameliorated symptoms in asthma, multiple sclerosis, and acute lung injury mouse models compared to those mouse models expressing native integrin β8.

### Cancer

Further, the antibodies described herein can be used to treat or prevent cancer. In some embodiments, the cancer includes cells that express αvβ8 on the cell surface or tissues composed of cells that express αvβ8. In certain embodiments, the cancer includes tumor cells that express αvβ8 on the cell surface. In some embodiments, the antibodies described herein bind to αvβ8 expressed on cancer cells and block ligand binding to αvβ8 to treat or prevent cancer. In certain embodiments, antibodies described herein can reduce tumor size, number of cancer cells, growth rate of cancer cells, metastatic activity of cancer cells, and/or cell death of non-cancer cells. In some embodiments, antibodies described herein can be used to reduce or prevent cancer metastasis.

Moreover, the antibodies described herein can enhance the effectiveness of immunomodulators, such as checkpoint antagonists and checkpoint agonists, and other non-drug based immunotherapies, such as radiotherapy. Examples of checkpoint antagonists include, but are not limited to, PD1 antagonists (e.g., RMP1-14, pembrolizumab, nivolumab, cemiplimab, JTX-4014, spartalizumab (PDR001), camrelizumab (SHR1210), sintilimab (1BI308), tislelizumab (BGB-A317), toripalimab (JS 001), dostarlimab (TSR-042, WBP-285), INCMGA00012 (MGA012), AMP-224, AMP-514), PDL1 antagonists (e.g., atezolizumab, avelumab, durvalumab, KN035, CK-301, AUNP12, CA-170, BMS-986189), and CTLA4 antagonists (e.g., 9D9, ipilimumab, tremelimumab).

Examples of checkpoint agonists include, but are not limited to, 41BB agonists (e.g., MAB9371, utomilumab, urelumab).

In some embodiments, antibodies described herein can be used to treat or prevent cancer in combination with a checkpoint antagonist (e.g., a PD1 antagonist, a PDL1 antagonist, or a CTLA4 antagonist as described above). In some embodiments, antibodies described herein can be used to treat or prevent cancer in combination with a checkpoint agonist (e.g., a 41BB agonist as described above). Further, antibodies described herein can also be used in combination with a non-drug based immunotherapy, such as radiotherapy, to treat or prevent cancer.

In some embodiments, the antibodies described herein can also bind to αvβ8 expressed on immune cells, e.g., regulatory T cells. In some embodiments, αvβ8 blocks the function and/or development of immune cells (e.g., regulatory T cells) and the antibodies described herein can stimulate immunity to cancer cells.

Cancers that can be treated by antibodies described herein include precancerous, neoplastic, transformed, and cancerous cells, and can refer to a solid tumor, or a non-solid cancer (see, e.g., Edge et al. AJCC Cancer Staging Manual (7^{th} ed. 2009); Cibas and Ducatman Cytology: Diagnostic principles and clinical correlates (3^{rd} ed. 2009)). Cancers can include both benign and malignant neoplasms (abnormal growth). Moreover, cancers can include carcinomas, sarcomas, adenocarcinomas, lymphomas, leukemias, solid and lymphoid cancers, etc. Examples of different types of cancer include, but are not limited to, lung cancer (e.g., non-small cell lung cancer or NSCLC), ovarian cancer, prostate cancer, colorectal cancer, liver cancer (i.e., hepatocarcinoma), renal cancer (i.e., renal cell carcinoma), bladder cancer, breast cancer, thyroid cancer, pleural cancer, pancreatic cancer, uterine cancer, cervical cancer, testicular cancer, anal cancer, pancreatic cancer, bile duct cancer, gastrointestinal carcinoid tumors, esophageal cancer, gall bladder cancer, appendix cancer, small intestine cancer, stomach (gastric) cancer, cancer of the central nervous system, skin cancer, choriocarcinoma; head and neck cancer, blood cancer, osteogenic sarcoma, fibrosarcoma, neuroblastoma, glioma, melanoma, B-cell lymphoma, non-Hodgkin's lymphoma, Burkitt's lymphoma, Small Cell lymphoma, Large Cell lymphoma, monocytic leukemia, myelogenous leukemia, acute lymphocytic leukemia, acute myelocytic leukemia (AML), chronic myeloid leukemia (CML), and multiple myeloma.

### VI. Pharmaceutical Composition and Administration

The antibodies can be provided in a pharmaceutical composition. The pharmaceutical compositions of the disclosure may comprise a pharmaceutically acceptable carrier. Pharmaceutically acceptable carriers are determined in part by the particular composition being administered, as well as by the particular method used to administer the composition. Accordingly, there are a wide variety of suitable formulations of pharmaceutical compositions of the present disclosure (see, e.g., Remington's Pharmaceutical Sciences, 17th ed., 1989).

Formulations suitable for administration include aqueous and non-aqueous solutions, isotonic sterile solutions, which can contain antioxidants, buffers, bacteriostats, and solutes that render the formulation isotonic, and aqueous and non-aqueous sterile suspensions that can include suspending agents, solubilizers, thickening agents, stabilizers, and preservatives. In the practice of this disclosure, compositions can be administered, for example, orally, nasally, topically, intravenously, intraperitoneally, or intrathecally. The formulations of compounds can be presented in unit-dose or multi-dose sealed containers, such as ampoules and vials. Solutions and suspensions can be prepared from sterile powders, granules, and tablets of the kind previously described. The modulators can also be administered as a part of prepared food or drug.

The dose administered to a patient should be sufficient to effect a beneficial response in the subject over time. The optimal dose level for any patient will depend on a variety of factors including the efficacy of the antibody employed, the age, body weight, physical activity, and diet of the patient, and on a possible combination with other drugs. The dose also will be determined by the existence, nature, and extent of any adverse side-effects that accompany the administration of a particular compound or vector in a particular subject.

In determining the effective amount of the antibody antagonists of αvβ8 integrin to be administered a physician may evaluate circulating plasma levels of the antagonist and antagonist toxicity. In general, the dose equivalent of an antagonist is from about 1 ng/kg to 10 mg/kg for a typical subject. In some embodiments, the dose range for sub-cutaneous or iv administration is 0.1-20, e.g., 0.3-10 mg/kg.

For administration, the antagonists of αvβ8 integrin can be administered at a rate determined by the LD₅₀ of the antagonist, and the side-effects of the antagonist at various concentrations, as applied to the mass and overall health of the subject. Administration can be accomplished via single or divided doses.

The compositions may be administered on a regular basis (e.g., daily) for a period of time (e.g., 2, 3, 4, 5, 6, days or 1-3 weeks or more). Formulations comprising the antibodies described herein can depend on how the formulations will be administered.

### Examples

### Example 1—Inhibition of L229 Cell Adhesion

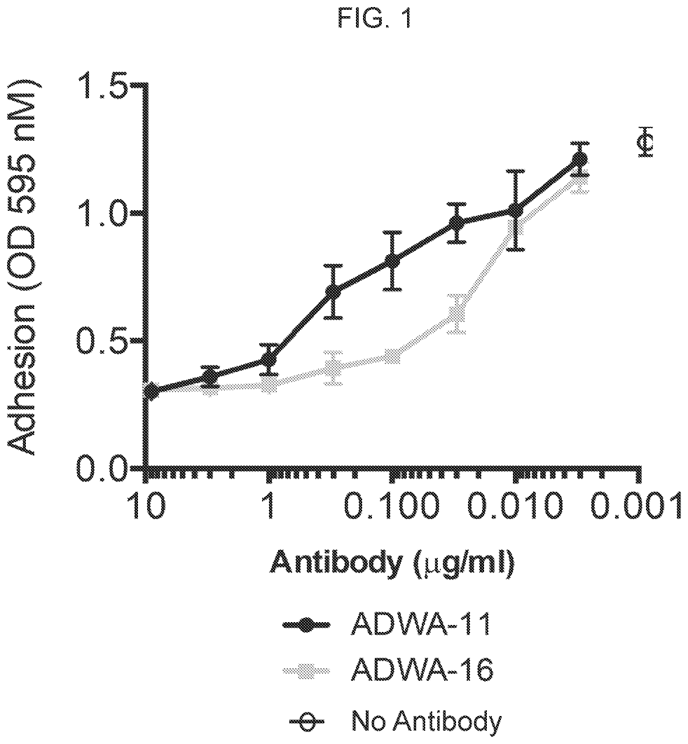

96-well tissue culture plates were coated with 1 μg/ml LAP in PBS, incubated at 37° C. for 1 hr. Wells were blocked with 2% BSA for an additional 1 hr at 37° C. SNB19 cells were plated at 50 k cells/well. For blocking conditions, cells were incubated with the indicated antibody for 10 min at 4° C. before final plating. Non-adherent cells were removed by centrifugation at 500 rpm for 5 min. Remaining adherent cells were stained using 0.5% crystal violet. Relative number of cells was determined after solubilization in 2% Triton X-100. All determinations were carried out in triplicate. As shown in FIG. 1, ADWA16 was more than 10-fold more potent at inhibiting adhesion of L229 cells expressing αvβ8 to its principal ligand, TGFb1 LAP, than was ADWA11.

### Example 2—Binding Competition

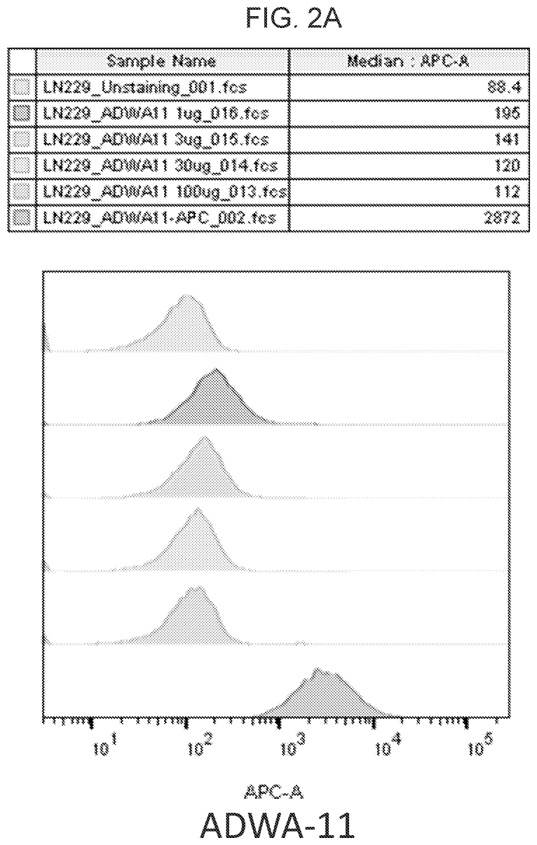

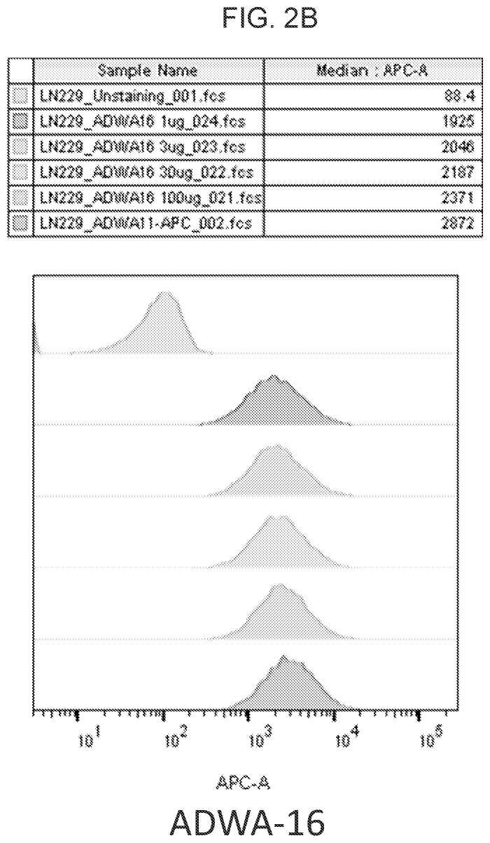

LN-229 cells were collected from 10 cm dishes and re-suspended in PBS. Cells were initially blocked with primary antibodies against β8 (ADWA11, ADWA16) at 1 μg, 3 μg, 30 μg, and 100 μg. Cells were washed with PBS before subsequent incubation with APC-conjugated ADWA11 at a final 1:500 dilution. Cells were analyzed on BD FACSCantoll for APC expression. As shown in FIGS. 2A and 2B, unlabeled ADWA11 effectively competed for binding of labelled ADWA11 to L229 cells expressing αvβ8, but unlabeled ADWA16 did not, demonstrating that the two antibodies recognize different epitopes.

### Example 3—Antibody Binding to αvβ8

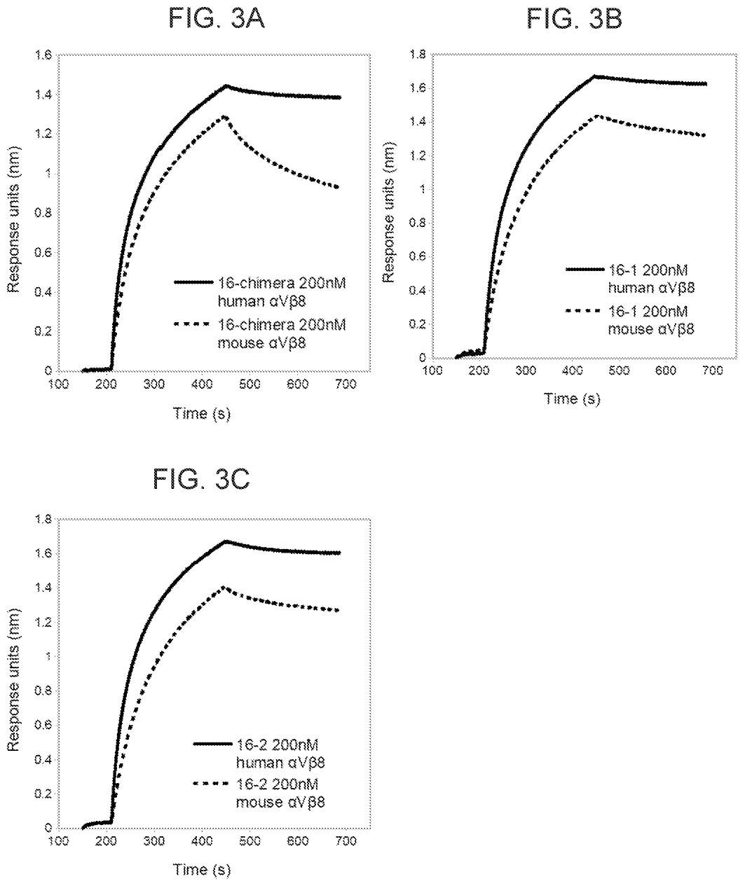

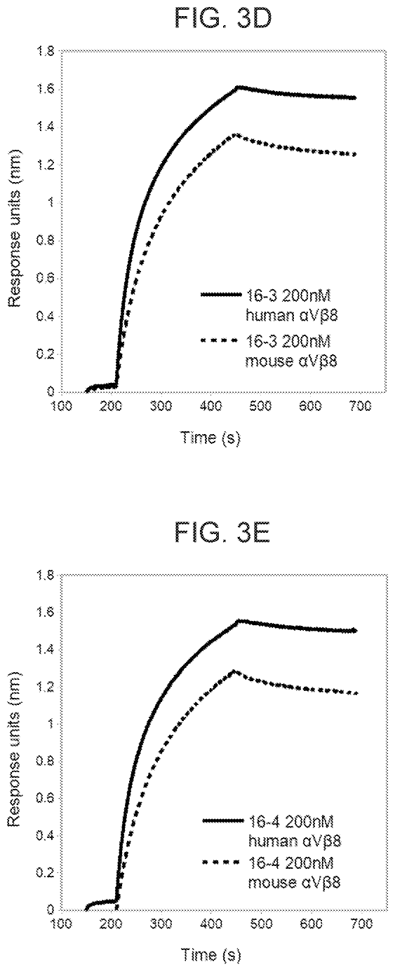

The affinities of humanized, affinity-matured anti-αvβ8 IgGs (ADWA16-1, ADWA16-2, ADWA16-3, and ADWA16-4) and a chimeric version of the parent ADWA-16 murine IgG (ADWA16-chimera) were measured for recombinant human and mouse αvβ8 using bio-layer interferometry. Anti-human Fab-CH1 tips were loaded with humanized anti-αvβ8 IgGs (ADWA16-1, ADWA16-2, ADWA16-3, and ADWA16-4) or chimeric ADWA-16 followed by an association step with either human or mouse αvβ8 (200 nM) and a subsequent dissociation step. All steps were carried out in binding buffer (25 mM Tris, 0.15 M NaCl, 0.05% Tween-20, pH 7.5). Binding affinities (shown in Table 2) were calculated using curve-fitting software. FIGS. 3A-3E show association/dissociation curves for each antibody. As can be seen in FIGS. 3A-3E, the humanized, affinity-matured IgGs (ADWA16-1, ADWA16-2, ADWA16-3, and ADWA16-4) showed affinity for recombinant human αvβ8 that was equivalent to the parent murine IgG (ADWA16-chimera) and improved affinity for recombinant mouse αvβ8.

TABLE 2

| IgG | Human αvβ8 KD (nM) | Mouse αvβ8 KD (nM) |
|---|---|---|
| ADWA16-chimera | 0.95 | 21 |
| ADWA16-1 | 0.73 | 4.8 |
| ADWA16-2 | 2.0 | 8.5 |
| ADWA16-3 | 0.83 | 5.0 |
| ADWA16-4 | 0.86 | 8.0 |

### Example 4—Antibody Binding to Human SNB19 Astrocytoma Cells Expressing αvβ8

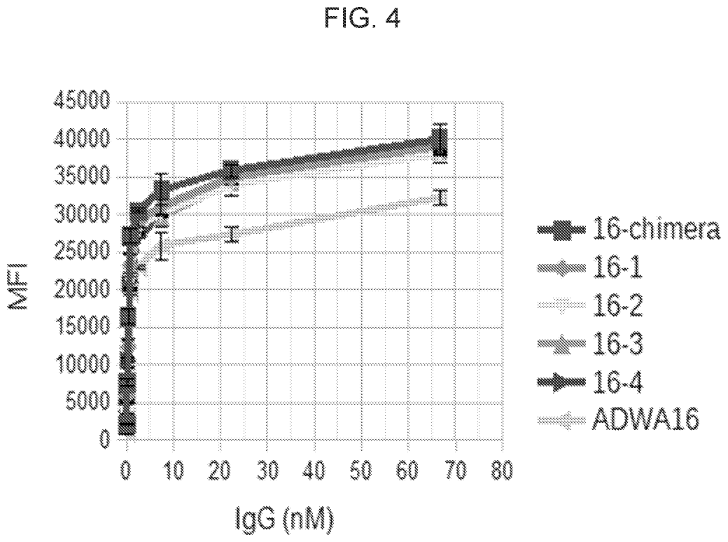

The affinities of humanized, affinity-matured anti-αvβ8 IgGs (ADWA16-1, ADWA16-2, ADWA16-3, and ADWA16-4), a chimeric version of the parent ADWA16 murine IgG (ADWA16-chimera), and ADWA16 were measured on SNB19 human astrocytoma cells by FACS. IgGs were incubated at various concentrations with SNB19 cells for 1 hr at RT in PBS. Cells were washed twice with PBS and binding was detected by incubation with fluorescently-labeled anti-human (ADWA16-1, ADWA16-2, ADWA16-3, ADWA16-4, and ADWA16-chimera) or anti-mouse (ADWA16) secondary antibodies followed by two PBS wash steps and analysis by FACS. Binding affinities (shown in Table 3) were calculated using curve-fitting software. As can be seen in FIG. 4, humanized, affinity-matured IgGs (ADWA16-1, ADWA16-2, ADWA16-3, and ADWA16-4) showed affinity for cell surface-expressed human αvβ8 that was equivalent to the parent murine IgG (ADWA16-chimera and ADWA16).

TABLE 3

| | ADWA16 | 16-chimera | 16-1 | 16-2 | 16-3 | 16-4 |
|---|---|---|---|---|---|---|
| KD | 417 | 302 | 459 | 498 | 389 | 614 |
| (pM) | | | | | | |

### Example 5—Removal of N-Glycosylation Site

In silico analysis of the ADWA16 heavy chain CDR2 revealed a potential N-glycosylation site (bold in the sequence of YINPTTGYTE (SEQ ID NO:2)). Yeast surface display of the ADWA16-3 scFv was used to screen amino acid substitutions at the predicted potential N-glycosylation site in the heavy chain CDR2. It was found that N to S substitution of the N-glycosylation site resulted in similar binding to human αvβ8 and improved binding (relative to 16-3) to mouse αvβ8.

### Example 6—ADWA16-3, ADWA16-3.2, and ADWA16-Chimera Binding to αvβ8

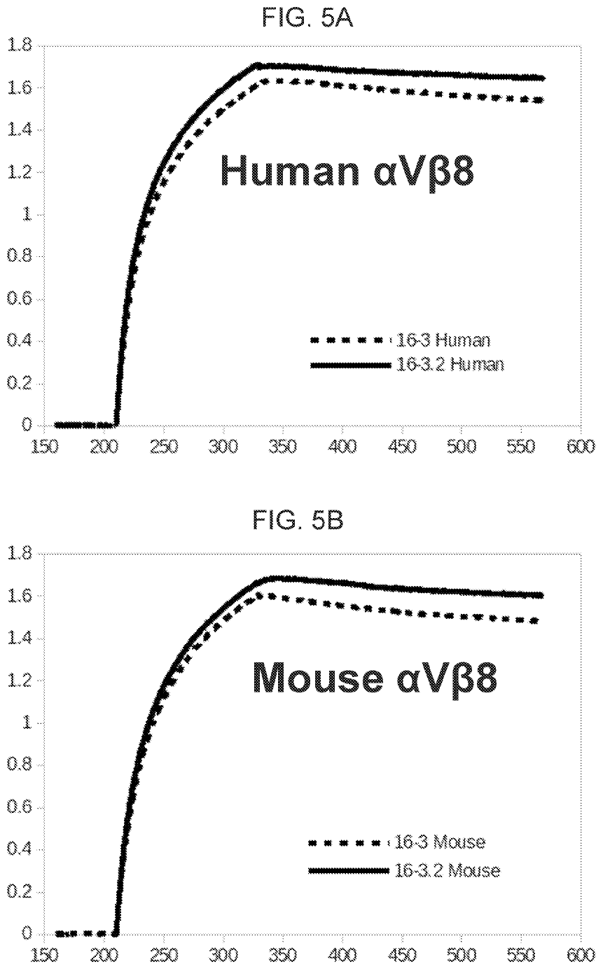

The affinities of humanized, affinity-matured anti-αvβ8 IgGs (ADWA16-3 and ADWA16-3.2) and a chimeric version of the parent ADWA16 murine IgG (ADWA16-chimera) were measured for recombinant human and mouse αvβ8 using bio-layer interferometry. Anti-human Fab-CH1 tips were loaded with humanized anti-αvβ8 IgGs or chimeric ADWA16 followed by an association step with either human or mouse αvβ8 (200 nM) and a subsequent dissociation step. All steps were carried out in binding buffer (25 mM Tris, 0.15 M NaCl, 0.05% Tween-20, pH 7.5). Binding affinities (shown in Table 4) were calculated using curve-fitting software. As shown in FIGS. 5A and 5B, the humanized, affinity-matured, and N-glycosylation site removed IgG (ADWA16-3.2) showed affinity for recombinant human αvβ8 that was equivalent to the parent murine IgG (ADWA16-chimera) and significantly improved affinity for recombinant mouse αvβ8. The affinity of ADWA16-3.2 was also improved relative to ADWA16-3.

TABLE 4

| | Human αVβ8 | Mouse αVβ8 |
|---|---|---|
| IgG | KD (pM) | KD (pM) |
| 16-3 | 1161 | 3154 |
| 16-3.2 | 688 | 1063 |
| 16-chimera | 950 | 21000 |

### Example 7—ADWA16-3 and ADWA16-3.2 Human SNB19 Astrocytoma Cells Expressing αvβ8

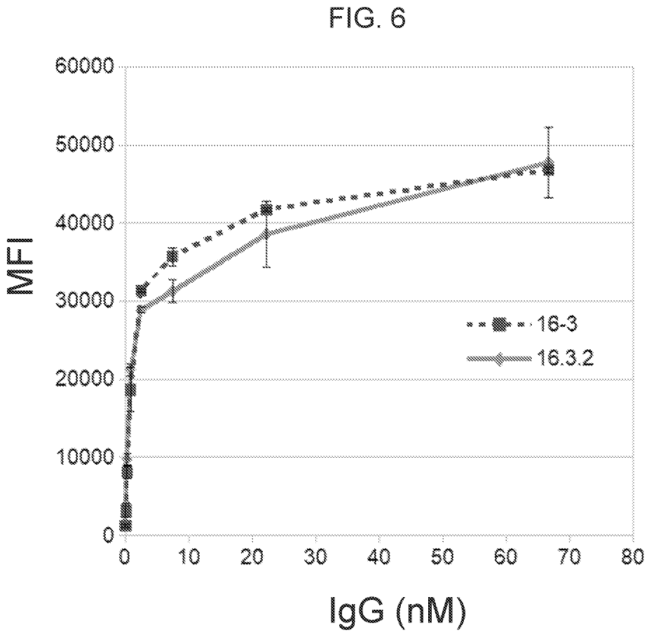

The affinities of humanized, affinity-matured, and N-glycosylation site removed anti-αvβ8 IgGs (ADWA16-3 and ADWA16-3.2) were measured on SNB19 human astrocytoma cells by FACS. IgGs were incubated at various concentrations with SNB19 cells for 1 hr at RT in PBS. Cells were washed twice with PBS and binding was detected by incubation with fluorescently-labeled anti-human secondary antibodies followed by two PBS wash steps and analysis by FACS. ADWA16-3 showed a binding affinity of 830 μM and ADWA16-3.2 showed a binding affinity of 512 pm. As can be seen in FIG. 6, the humanized, affinity-matured and N-glycosylation site removed anti-αvβ8 IgGs (ADWA16-3 and ADWA16-3.2) displayed affinity for cell surface-expressed human αvβ8 that was equivalent to the parent murine IgG (ADWA16-chimera and ADWA16).

### Example 8—Antibody Binding to Binding to Murine Astrocytes

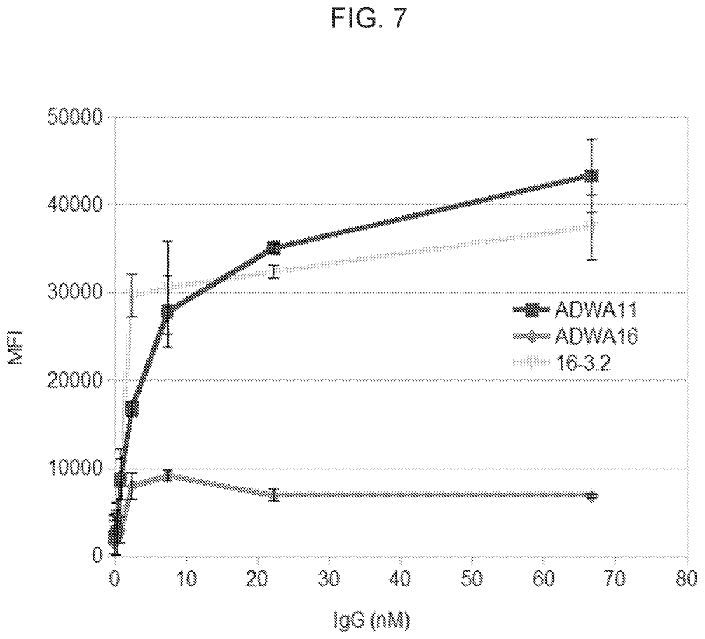

The affinities of ADWA16, ADWA11, and ADWA16-3.2 were measured on murine astrocytes by FACS. IgGs were incubated at various concentrations with the cells for 1 hr at RT in PBS. Cells were washed twice with PBS and binding was detected by incubation with fluorescently-labeled anti-human or anti-mouse secondary antibodies followed by two PBS wash steps and analysis by FACS. ADWA16-3.2 showed a binding affinity of 1.1 nM and ADWA11 showed a binding affinity of 5.6 nM. As shown in FIG. 7, ADWA16-3.2 IgG displayed significantly improved binding to surface-expressed mouse αvβ8 compared to the parent murine IgG (ADWA16).

### Example 9—Binding Specificity

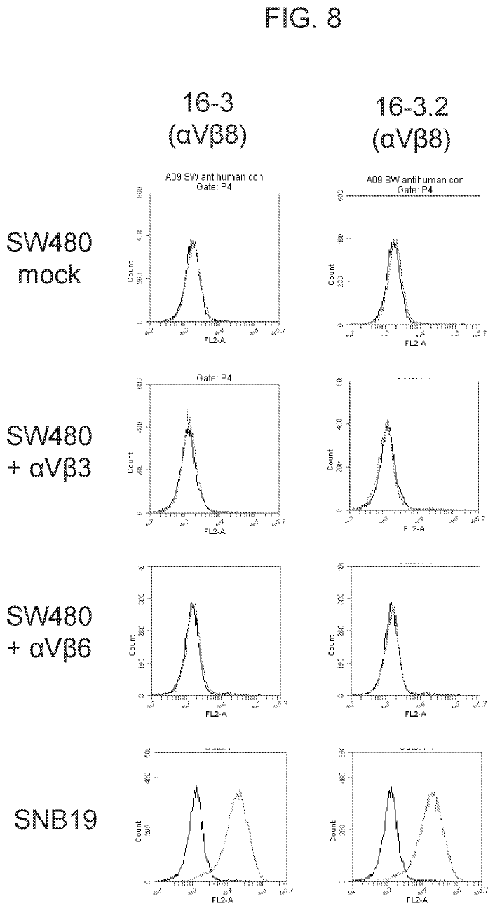

The binding of ADWA16 and ADWA16-3.2 to cell surface expressed human αvβ3, αvβ5, αvβ6, and αvβ8 was tested by FACS. Flow cytometry was performed with either ADWA16-3 or ADWA16-3.2 with mock transfected SW480 cells (that express only αvβ5 integrin). SW480 cells were also transfected to express αvβ3 or αvβ6. SNB19 cells expressed αvβ3, αvβ5, and αvβ8. As shown in FIG. 8, ADWA16 and ADWA16-3.2 did not bind to αvβ3, αvβ5, or αvβ6-expressing SW480 cells, but only displayed binding to SNB19 cells expressing αvβ3, αvβ5, and αvβ8.

### Example 10—Inhibition of TGFβ Activation

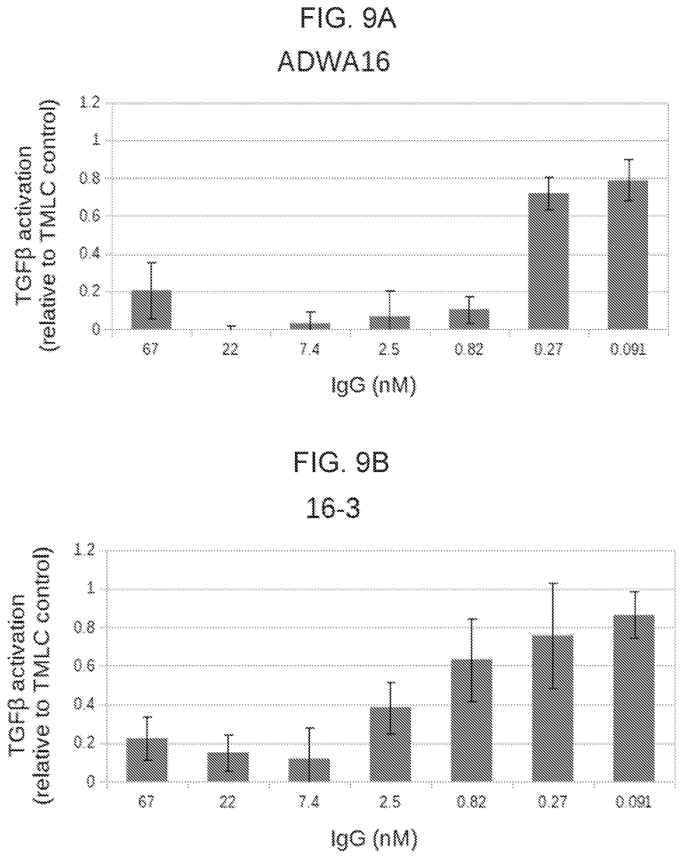

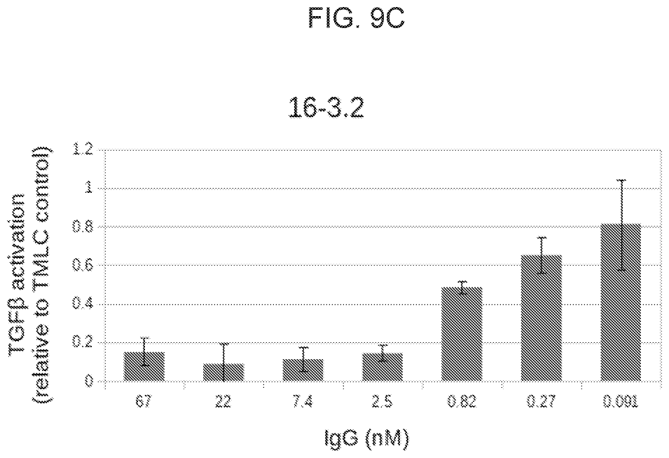

SNB19 cells, that express αvβ8 as their only TGFβ activating integrin, were co-cultured overnight in 96-well tissue culture plates with mink lung reporter cells transfected to express a TGFβ responsive portion of the PAI-1 promoter driving expression of firefly luciferase (TMLC cells). A range of dilutions of either ADWA16, ADWA16-3, or ADWA16-3.2 was added at the start of each experiment. Cells were lysed and luciferase activity was measured and plotted as a fraction of luciferase activity from co-culture of TMLCs and SNB19 cells without antibody. All determinations were carried out in triplicate. Error bars show +/−SEM. ADWA16 displayed an IC₅₀ of 364 μM; ADWA16-3 displayed an IC₅₀ of 1360 μM; and ADWA16-3.2 displayed an IC₅₀ of 580 μM. As shown in FIGS. 9A-9C, ADWA16-3 and ADWA16-3.2 retained the high potency of the murine parent ADWA16 to inhibit activation of TGFβ.

### Example 11—Inhibition of SNB19 Cell Adhesion to TGFβ1-LAP

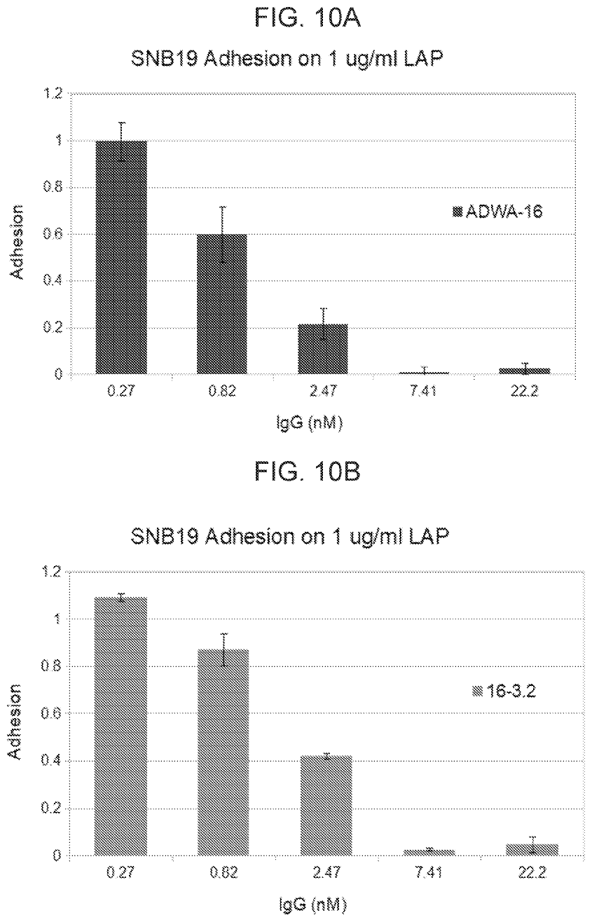

96-well tissue culture plates were coated with 1 μg/ml LAP in PBS, incubated at 37° C. for 1 hr. Wells were blocked with 2% BSA for an additional 1 hr at 37° C. SNB19 cells were plated at 50 k cells/well. For blocking conditions, cells were incubated with indicated antibody for 10 min at 4° C. before final plating. Non-adherent cells were removed by centrifugation at 500 rpm for 5 min. Remaining adherent cells were stained using 0.5% crystal violet. Cell adhesion was quantified by absorbance after solubilization in 2% Triton X-100. All determinations were carried out in triplicate. Error bars show +/−SEM. ADWA16 displayed an IC₅₀ of 1.1 nM; and ADWA16-3.2 displayed an IC₅₀ of 2.1 nM. As shown in FIGS. 10A and 10B, ADWA16-3.2 retained low nanomolar potency in inhibiting adhesion of SNB19 cells to TGFβ1-LAP.

### Example 12—Thermal Stability

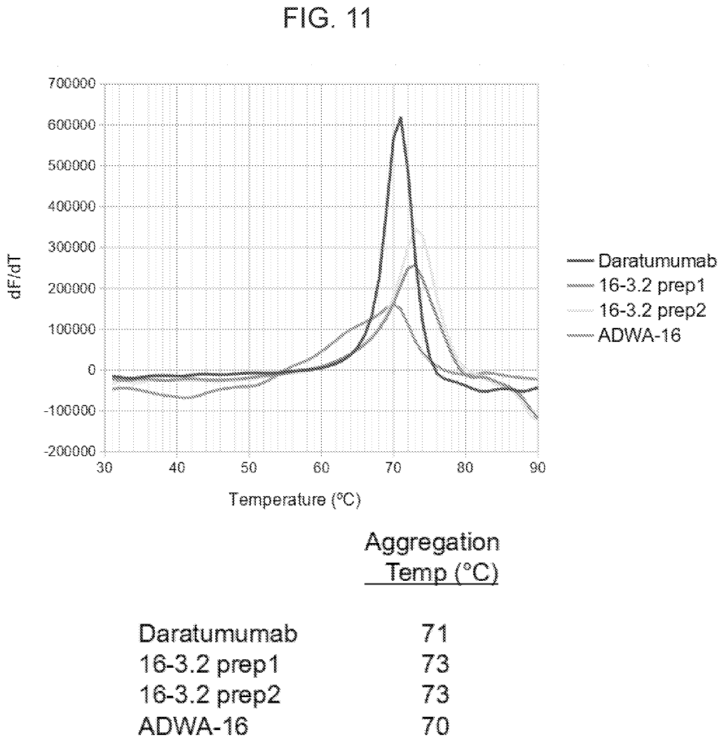

A molecular rotor dye that binds to the surface of protein aggregates was mixed with Daratumumab, ADWA16-3.2, and ADWA16 antibodies and a real-time PCR instrument was programmed to ramp the temperature from 30° C. to 90° C. at a 3° C./minute rate while reading the fluorescence continuously. A first derivative plot was used to calculate the aggregation temperature of the antibodies. FIG. 11 shows that ADWA16-3.2 displayed improved thermal stability compared to the parent murine antibody ADWA-16.

### Example 13—Antibody Binding to Human Astrocytoma Line SNB19

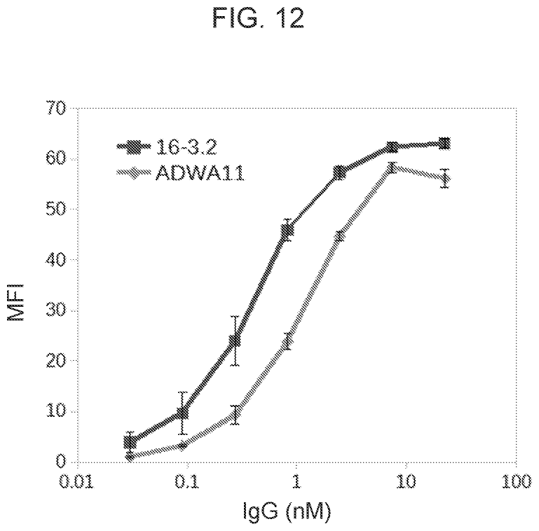

The affinities of ADWA16-3.2 and ADWA11 were measured on SNB19 human astrocytoma cells by FACS. IgGs were incubated at various concentrations with SNB19 cells for 1 hour at RT in PBS. Cells were washed twice with PBS and binding was detected by incubation with fluorescently-labeled anti-human secondary antibodies followed by two PBS wash steps and analysis by FACS. Binding affinities were calculated using curve-fitting software. ADWA16-3.2 and ADWA11 displayed K_{D} values of 392 μM and 1040 μM, respectively. Binding curves are shown in FIG. 12. As demonstrated, ADWA16-3.2 displayed greater affinity for cell surface-expressed human αvβ8 than ADWA11.

### Example 14—Inhibition of SNB19 Cell Adhesion to TGFb1 LAP

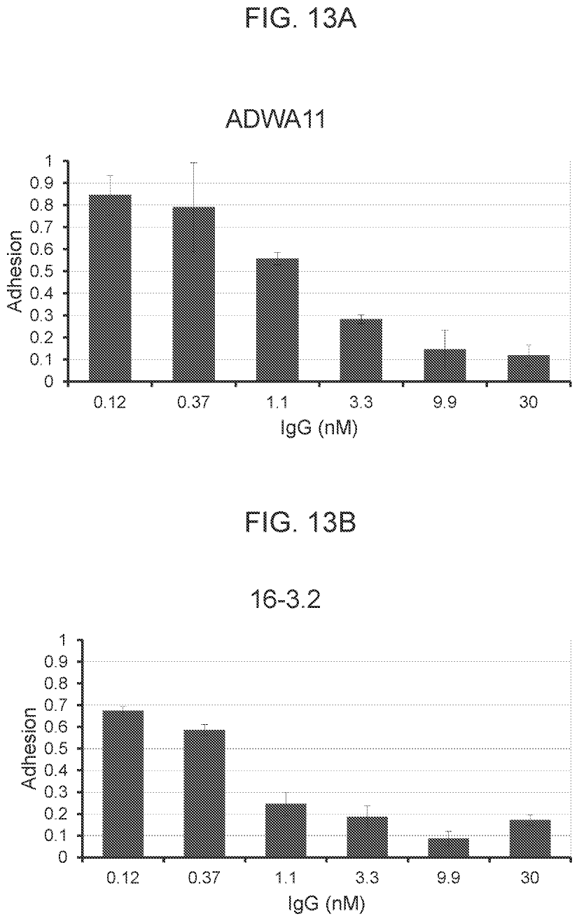

96-well tissue culture plates were coated with 1 μg/ml LAP in PBS, incubated at 37° C. for 1 hr. Wells were blocked with 2% BSA for an additional 1 hr at 37° C. SNB19 cells were plated at 50 k cells/well. For blocking conditions, cells were incubated with indicated antibody for 10 min at 4° C. before final plating. Non-adherent cells were removed by centrifugation at 500 rpm for 5 min. Remaining adherent cells were stained using 0.5% crystal violet. Cell adhesion was quantified by absorbance after solubilization in 2% Triton X-100. All determinations were carried out in triplicate. Error bars show +/−SEM. As shown in FIGS. 13A and 13B, ADWA16-3.2, the humanized derivative of ADWA16, inhibited adhesion of SNB19 cells to TGFb1 LAP with greater potency than ADWA11. ADWA11 displayed an IC₅₀ of 1435 μM; ADWA16-3.2 displayed an IC₅₀ of 650 μM.

The above examples are provided to illustrate the disclosure but not to limit its scope. Other variants of the disclosure will be readily apparent to one of ordinary skill in the art and are encompassed by the appended claims. All publications, databases, internet sources, patents, patent applications, and accession numbers cited herein are hereby incorporated by reference in their entireties for all purposes.

What is claimed is:

1. An isolated antibody that specifically binds to human integrin β8 and inhibits adhesion of latency associated peptide (LAP) to αvβ8, wherein the isolated antibody comprises:
(1) a heavy chain complementary determining region 1 (HCDR1) having a sequence of any one of SEQ ID NOS:1, 5, and 6;
(2) an HCDR2 having a sequence of any one of SEQ ID NOS:2, 4, and 7;
(3) an HCDR3 having the sequence of SEQ ID NO:3;
(4) a light chain complementary determining region 1 (LCDR1) having a sequence of any one of SEQ ID NOS:8, 11, 13, and 14;
(5) a LCDR2 having a sequence of any one of SEQ ID NOS:9 and 12; and
(6) a LCDR3 having the sequence of SEQ ID NO:10.

2. The isolated antibody of claim 1, wherein the antibody comprises an HCDR1 having the sequence of SEQ ID NO:1, an HCDR2 having the sequence of SEQ ID NO: 2, and an HCDR3 having the sequence of SEQ ID NO: 3.

3. The isolated antibody of claim 1, wherein the antibody comprises an HCDR1 having the sequence of SEQ ID NO:1, an HCDR2 having the sequence of SEQ ID NO: 4, and an HCDR3 having the sequence of SEQ ID NO: 3.

4. The isolated antibody of claim 1, wherein the antibody comprises an HCDR1 having the sequence of SEQ ID NO: 5, an HCDR2 having the sequence of SEQ ID NO: 2, and an HCDR3 having the sequence of SEQ ID NO: 3.

5. The isolated antibody of claim 1, wherein the antibody comprises an HCDR1 having the sequence of SEQ ID NO: 6, an HCDR2 having the sequence of SEQ ID NO: 2, and an HCDR3 having the sequence of SEQ ID NO: 3.

6. The isolated antibody of claim 1, wherein the antibody comprises an HCDR1 having the sequence of SEQ ID NO:1, an HCDR2 having the sequence of SEQ ID NO: 7, and an HCDR3 having the sequence of SEQ ID NO: 3.

7. The isolated antibody of any one of claims 1 to 6, wherein the antibody comprises a LCDR1 having the sequence of SEQ ID NO: 8, a LCDR2 having the sequence of SEQ ID NO: 9, and a LCDR3 having the sequence of SEQ ID NO: 10.

8. The isolated antibody of any one of claims 1 to 6, wherein the antibody comprises a LCDR1 having the sequence of SEQ ID NO: 11, a LCDR2 having the sequence of SEQ ID NO: 9, and a LCDR3 having the sequence of SEQ ID NO: 10.

9. The isolated antibody of any one of claims 1 to 6, wherein the antibody comprises a LCDR1 having the sequence of SEQ ID NO: 8, a LCDR2 having the sequence of SEQ ID NO: 12, and a LCDR3 having the sequence of SEQ ID NO: 10.

10. The isolated antibody of any one of claims 1 to 6, wherein the antibody comprises a LCDR1 having the sequence of SEQ ID NO: 13, a LCDR2 having the sequence of SEQ ID NO: 12, and a LCDR3 having the sequence of SEQ ID NO: 10.

11. The isolated antibody of any one of claims 1 to 6, wherein the antibody comprises a LCDR1 having the sequence of SEQ ID NO: 14, a LCDR2 having the sequence of SEQ ID NO: 9, and a LCDR3 having the sequence of SEQ ID NO: 10.

12. The isolated antibody of any one of claims 1 to 11, wherein the antibody comprises a heavy chain variable region having at least 90% identity to a sequence of any one of SEQ ID NOS:15-19.

13. The isolated antibody of any one of claims 1 to 12, wherein the antibody comprises a light chain variable region having at least 90% identity to a sequence of any one of SEQ ID NOS:20-25.

14. The isolated antibody of any one of claims 1 to 13, wherein the antibody comprises an Fc polypeptide having at least 90% identity to a sequence of any one of SEQ ID NOS:47-50.

15. The isolated antibody of claim 14, wherein the Fc polypeptide comprises amino acid substitutions L234A and L235A.

16. The isolated antibody of claim 14 or 15, wherein the Fc polypeptide comprises the amino acid substitution N297A.

17. The isolated antibody of claim 1, wherein the antibody comprises:
(1) an HCDR1 having the sequence of SEQ ID NO: 1;
(2) an HCDR2 having the sequence of SEQ ID NO: 4;
(3) an HCDR3 having the sequence of SEQ ID NO: 3;
(4) a LCDR1 having the sequence of SEQ ID NO: 8;
(5) a LCDR2 having the sequence of SEQ ID NO: 12; and
(6) a LCDR3 having the sequence of SEQ ID NO: 10.

18. The isolated antibody of claim 17, wherein the antibody comprises a heavy chain variable region having at least 90% identity to the sequence of SEQ ID NO: 16.

19. The isolated antibody of claim 17 or 18, wherein the antibody comprises a light chain variable region having at least 90% identity to the sequence of SEQ ID NO: 22.

20. The isolated antibody of any one of claims 17 to 19, wherein the antibody comprises an Fc polypeptide having at least 90% identity to a sequence of any one of SEQ ID NOS:47-50.

21. The isolated antibody of claim 20, wherein the Fc polypeptide comprises amino acid substitutions L234A and L235A.

22. The isolated antibody of claim 19 or 20, wherein the Fc polypeptide comprises the amino acid substitution N297A.

23. The isolated antibody of any one of claims 1 to 22, wherein the antibody is a monoclonal antibody.

24. The isolated antibody of any one of claims 1 to 23, wherein the antibody is a humanized antibody.

25. The isolated antibody of any one of claims 1 to 24, wherein the antibody cross-reacts with mouse integrin β8.

26. The isolated antibody of any one of claims 1 to 25, wherein the antibody blocks TGFβ activation.

27. The isolated antibody of any one of claims 1 to 26, wherein the antibody antagonizes binding of LAP to αvβ8 with an IC₅₀ below 5 nM.

28. The isolated antibody of any one of claims 1 to 27, wherein the antibody comprises one or more human framework regions.

29. An isolated nucleic acid encoding the isolated antibody of any one of claims 1 to 28.

30. An expression vector comprising the nucleic acid of claim 29.

31. An isolated host cell comprising the vector of claim 30.

32. A pharmaceutical composition comprising the isolated antibody of any one of claim 1 to 28 and a pharmaceutically acceptable carrier.

33. A method of reducing TGFβ activation in a human in need thereof, the method comprising administering a therapeutically effective amount of the isolated antibody of any of claims 1 to 28 or the pharmaceutical composition of claim 32 to the human, thereby reducing TGFβ activation in the human.

34. The method of claim 33, wherein the human has a disease selected from the group consisting of asthma, multiple sclerosis or acute lung injury and at least one symptom of the disease is ameliorated by the reduced TGFβ activation.

35. The method of claim 33, wherein the human has a disease selected from the group consisting of rheumatoid arthritis, psoriasis and chronic obstructive pulmonary disease and at least one symptom of the disease is ameliorated by the reduced TGFβ activation.

36. A method of treating a cancer in a human, the method comprising administering to the human a therapeutically effective amount of the isolated antibody of any of claims 1 to 28 or the pharmaceutical composition of claim 32 to the human, thereby treating the cancer.

37. The method of claim 36, wherein the cancer is a metastatic cancer.

38. The method of claim 36 or 37, wherein the cancer is a solid tumor cancer.

39. The method of any one of claims 36 to 38, wherein the method enhances an immune response to the cancer in the human.

40. The method of any one of claims 36 to 39, wherein the method further comprises administering an immunomodulator to the human.

41. The method of claim 40, wherein the immunomodulator is a PD1 antagonist, a PDL1 antagonist, a CTLA4 antagonist, or a 41BB agonist.

42. The method of any one of claims 36 to 41, wherein the method further comprises administering radiotherapy to the human.


SEQUENCE LISTING (58 sequences)

SEQ ID NO: 1 - protein, 13 aa, Artificial Sequence
  Description of Artificial Sequence Synthetic peptide
AVSGYIFSSYWVY

SEQ ID NO: 2 - protein, 10 aa, Artificial Sequence
  Description of Artificial Sequence Synthetic peptide
  MOD_RES (3)..(3): N, S, I or V
  See specification as filed for detailed description of substitutions and preferred embodiments
YIXPTTGYTE

SEQ ID NO: 3 - protein, 10 aa, Artificial Sequence
  Description of Artificial Sequence Synthetic peptide
ATEGGNWEDY

SEQ ID NO: 4 - protein, 10 aa, Artificial Sequence
  Description of Artificial Sequence Synthetic peptide
YISPTTGYTV

SEQ ID NO: 5 - protein, 13 aa, Artificial Sequence
  Description of Artificial Sequence Synthetic peptide
AASGYTFSSYWIY

SEQ ID NO: 6 - protein, 13 aa, Artificial Sequence
  Description of Artificial Sequence Synthetic peptide
KASGYTFSSYWIY

SEQ ID NO: 7 - protein, 10 aa, Artificial Sequence
  Description of Artificial Sequence Synthetic peptide
YISPTTGYTE

SEQ ID NO: 8 - protein, 11 aa, Artificial Sequence
  Description of Artificial Sequence Synthetic peptide
RASQDISNYIN

SEQ ID NO: 9 - protein, 8 aa, Artificial Sequence
  Description of Artificial Sequence Synthetic peptide
YYTSRLYS

SEQ ID NO: 10 - protein, 9 aa, Artificial Sequence
  Description of Artificial Sequence Synthetic peptide
QQFSELPRT

SEQ ID NO: 11 - protein, 11 aa, Artificial Sequence
  Description of Artificial Sequence Synthetic peptide
RASKDISNYLN

SEQ ID NO: 12 - protein, 8 aa, Artificial Sequence
  Description of Artificial Sequence Synthetic peptide
YYTSRLYG

SEQ ID NO: 13 - protein, 11 aa, Artificial Sequence
  Description of Artificial Sequence Synthetic peptide
RASKDISNYIN

SEQ ID NO: 14 - protein, 11 aa, Artificial Sequence
  Description of Artificial Sequence Synthetic peptide
RASQDISNYLN

SEQ ID NO: 15 - protein, 117 aa, Artificial Sequence
  Description of Artificial Sequence Synthetic polypeptide
EVQLVESGGGLVQPGGSLRLSCAVSGYIFSSYWVYWVRQAPGKGLEWVGYINPTTGYTEYADSVKGRFTISRDNSKNTLYLQMNSLRAEDTAVYYCATEGGNWEDYWGQGTLVTVSS

SEQ ID NO: 16 - protein, 117 aa, Artificial Sequence
  Description of Artificial Sequence Synthetic polypeptide
EVQLVESGGGLVQPGGSLRLSCAVSGYIFSSYWVYWVRQAPGKGLEWVGYISPTTGYTVYADSVKGRFTISRDNSKNTLYLQMNSLRAEDTAVYYCATEGGNWEDYWGQGTLVTVSS

SEQ ID NO: 17 - protein, 117 aa, Artificial Sequence
  Description of Artificial Sequence Synthetic polypeptide
EVQLVESGGGLVQPGGSLRLSCAASGYTFSSYWIYWVRQAPGKGLEWVSYINPTTGYTEYADSVKGRFTISRDNSKNTLYLQMNSLRAEDTAVYYCATEGGNWEDYWGQGTLVTVSS

SEQ ID NO: 18 - protein, 117 aa, Artificial Sequence
  Description of Artificial Sequence Synthetic polypeptide
QVQLQQSGAELAKPGASMKMSCKASGYTFSSYWIYWVKQRPGQGLEWIGYINPTTGYTEYNQKFRDKATLTADKSSNTAYMQLSSLTSEDSAVYYCATEGGNWEDYWGQGTTLTVSS

SEQ ID NO: 19 - protein, 117 aa, Artificial Sequence
  Description of Artificial Sequence Synthetic polypeptide
EVQLVESGGGLVQPGGSLRLSCAVSGYIFSSYWVYWVRQAPGKGLEWVGYISPTTGYTEYADSVKGRFTISRDNSKNTLYLQMNSLRAEDTAVYYCATEGGNWEDYWGQGTLVTVSS

SEQ ID NO: 20 - protein, 107 aa, Artificial Sequence
  Description of Artificial Sequence Synthetic polypeptide
DIQMTQSPSSLSASVGDRVTITCRASQDISNYINWYQQKPGKAPKLLIYYTSRLYSGVPSRFSGSGSGTDFTLTISSLQPEDFATYYCQQFSELPRTFGQGTKVEIK

SEQ ID NO: 21 - protein, 107 aa, Artificial Sequence
  Description of Artificial Sequence Synthetic polypeptide
DIQMTQSPSSLSASVGDRVTITCRASKDISNYLNWYQQKPGKAPKLLIYYTSRLYSGVPSRFSGSGSGTDFTLTISSLQPEDFATYYCQQFSELPRTFGQGTKVEIK

SEQ ID NO: 22 - protein, 107 aa, Artificial Sequence
  Description of Artificial Sequence Synthetic polypeptide
DIQMTQSPSSLSASVGDRVTITCRASQDISNYINWYQQKPGKAPKLLIYYTSRLYGGVPSRFSGSGSGTDFTLTISSLQPEDFATYYCQQFSELPRTFGQGTKVEIK

SEQ ID NO: 23 - protein, 107 aa, Artificial Sequence
  Description of Artificial Sequence Synthetic polypeptide
DIQMTQSPSSLSASVGDRVTITCRASKDISNYINWYQQKPGKAPKLLIYYTSRLYGGVPSRFSGSGSGTDFTLTISSLQPEDFATYYCQQFSELPRTFGQGTKVEIK

SEQ ID NO: 24 - protein, 107 aa, Artificial Sequence
  Description of Artificial Sequence Synthetic polypeptide
DIQMTQSPSSLSASVGDRVTITCRASQDISNYLNWYQQKPGKAPKLLIYYTSRLYSGVPSRFSGSGSGTDFTLTISSLQPEDFATYYCQQFSELPRTFGQGTKVEIK

SEQ ID NO: 25 - protein, 107 aa, Artificial Sequence
  Description of Artificial Sequence Synthetic polypeptide
DIQMTQTTSSLSASLGDRVTISCRASQDISNYLNWYQQKPDGTVKLLIYYTSRLYSGVPSRFSGSGSGTDYSLTISNLEPKDIATYYCQQFSELPRTFGGGTKLEIK

SEQ ID NO: 26 - protein, 447 aa, Artificial Sequence
  Description of Artificial Sequence Synthetic polypeptide
EVQLVESGGGLVQPGGSLRLSCAVSGYIFSSYWVYWVRQAPGKGLEWVGYINPTTGYTEYADSVKGRFTISRDNSKNTLYLQMNSLRAEDTAVYYCATEGGNWEDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 27 - protein, 214 aa, Artificial Sequence
  Description of Artificial Sequence Synthetic polypeptide
DIQMTQSPSSLSASVGDRVTITCRASQDISNYINWYQQKPGKAPKLLIYYTSRLYSGVPSRFSGSGSGTDFTLTISSLQPEDFATYYCQQFSELPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 28 - protein, 447 aa, Artificial Sequence
  Description of Artificial Sequence Synthetic polypeptide
EVQLVESGGGLVQPGGSLRLSCAVSGYIFSSYWVYWVRQAPGKGLEWVGYINPTTGYTEYADSVKGRFTISRDNSKNTLYLQMNSLRAEDTAVYYCATEGGNWEDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 29 - protein, 447 aa, Artificial Sequence
  Description of Artificial Sequence Synthetic polypeptide
EVQLVESGGGLVQPGGSLRLSCAVSGYIFSSYWVYWVRQAPGKGLEWVGYINPTTGYTEYADSVKGRFTISRDNSKNTLYLQMNSLRAEDTAVYYCATEGGNWEDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 30 - protein, 214 aa, Artificial Sequence
  Description of Artificial Sequence Synthetic polypeptide
DIQMTQSPSSLSASVGDRVTITCRASKDISNYLNWYQQKPGKAPKLLIYYTSRLYSGVPSRFSGSGSGTDFTLTISSLQPEDFATYYCQQFSELPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 31 - protein, 214 aa, Artificial Sequence
  Description of Artificial Sequence Synthetic polypeptide
DIQMTQSPSSLSASVGDRVTITCRASQDISNYINWYQQKPGKAPKLLIYYTSRLYGGVPSRFSGSGSGTDFTLTISSLQPEDFATYYCQQFSELPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 32 - protein, 214 aa, Artificial Sequence
  Description of Artificial Sequence Synthetic polypeptide
DIQMTQSPSSLSASVGDRVTITCRASKDISNYINWYQQKPGKAPKLLIYYTSRLYGGVPSRFSGSGSGTDFTLTISSLQPEDFATYYCQQFSELPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 33 - protein, 447 aa, Artificial Sequence
  Description of Artificial Sequence Synthetic polypeptide
EVQLVESGGGLVQPGGSLRLSCAVSGYIFSSYWVYWVRQAPGKGLEWVGYISPTTGYTVYADSVKGRFTISRDNSKNTLYLQMNSLRAEDTAVYYCATEGGNWEDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 34 - protein, 447 aa, Artificial Sequence
  Description of Artificial Sequence Synthetic polypeptide
EVQLVESGGGLVQPGGSLRLSCAVSGYIFSSYWVYWVRQAPGKGLEWVGYISPTTGYTVYADSVKGRFTISRDNSKNTLYLQMNSLRAEDTAVYYCATEGGNWEDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 35 - protein, 447 aa, Artificial Sequence
  Description of Artificial Sequence Synthetic polypeptide
EVQLVESGGGLVQPGGSLRLSCAVSGYIFSSYWVYWVRQAPGKGLEWVGYISPTTGYTVYADSVKGRFTISRDNSKNTLYLQMNSLRAEDTAVYYCATEGGNWEDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 36 - protein, 447 aa, Artificial Sequence
  Description of Artificial Sequence Synthetic polypeptide
EVQLVESGGGLVQPGGSLRLSCAASGYTFSSYWIYWVRQAPGKGLEWVSYINPTTGYTEYADSVKGRFTISRDNSKNTLYLQMNSLRAEDTAVYYCATEGGNWEDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 37 - protein, 214 aa, Artificial Sequence
  Description of Artificial Sequence Synthetic polypeptide
DIQMTQSPSSLSASVGDRVTITCRASQDISNYLNWYQQKPGKAPKLLIYYTSRLYSGVPSRFSGSGSGTDFTLTISSLQPEDFATYYCQQFSELPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 38 - protein, 447 aa, Artificial Sequence
  Description of Artificial Sequence Synthetic polypeptide
EVQLVESGGGLVQPGGSLRLSCAASGYTFSSYWIYWVRQAPGKGLEWVSYINPTTGYTEYADSVKGRFTISRDNSKNTLYLQMNSLRAEDTAVYYCATEGGNWEDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 39 - protein, 447 aa, Artificial Sequence
  Description of Artificial Sequence Synthetic polypeptide
EVQLVESGGGLVQPGGSLRLSCAASGYTFSSYWIYWVRQAPGKGLEWVSYINPTTGYTEYADSVKGRFTISRDNSKNTLYLQMNSLRAEDTAVYYCATEGGNWEDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 40 - protein, 447 aa, Artificial Sequence
  Description of Artificial Sequence Synthetic polypeptide
QVQLQQSGAELAKPGASMKMSCKASGYTFSSYWIYWVKQRPGQGLEWIGYINPTTGYTEYNQKFRDKATLTADKSSNTAYMQLSSLTSEDSAVYYCATEGGNWEDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 41 - protein, 214 aa, Artificial Sequence
  Description of Artificial Sequence Synthetic polypeptide
DIQMTQTTSSLSASLGDRVTISCRASQDISNYLNWYQQKPDGTVKLLIYYTSRLYSGVPSRFSGSGSGTDYSLTISNLEPKDIATYYCQQFSELPRTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 42 - protein, 447 aa, Artificial Sequence
  Description of Artificial Sequence Synthetic polypeptide
QVQLQQSGAELAKPGASMKMSCKASGYTFSSYWIYWVKQRPGQGLEWIGYINPTTGYTEYNQKFRDKATLTADKSSNTAYMQLSSLTSEDSAVYYCATEGGNWEDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 43 - protein, 447 aa, Artificial Sequence
  Description of Artificial Sequence Synthetic polypeptide
QVQLQQSGAELAKPGASMKMSCKASGYTFSSYWIYWVKQRPGQGLEWIGYINPTTGYTEYNQKFRDKATLTADKSSNTAYMQLSSLTSEDSAVYYCATEGGNWEDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 44 - protein, 447 aa, Artificial Sequence
  Description of Artificial Sequence Synthetic polypeptide
EVQLVESGGGLVQPGGSLRLSCAVSGYIFSSYWVYWVRQAPGKGLEWVGYISPTTGYTEYADSVKGRFTISRDNSKNTLYLQMNSLRAEDTAVYYCATEGGNWEDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 45 - protein, 447 aa, Artificial Sequence
  Description of Artificial Sequence Synthetic polypeptide
EVQLVESGGGLVQPGGSLRLSCAVSGYIFSSYWVYWVRQAPGKGLEWVGYISPTTGYTEYADSVKGRFTISRDNSKNTLYLQMNSLRAEDTAVYYCATEGGNWEDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 46 - protein, 447 aa, Artificial Sequence
  Description of Artificial Sequence Synthetic polypeptide
EVQLVESGGGLVQPGGSLRLSCAVSGYIFSSYWVYWVRQAPGKGLEWVGYISPTTGYTEYADSVKGRFTISRDNSKNTLYLQMNSLRAEDTAVYYCATEGGNWEDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 47 - protein, 217 aa, Homo sapiens
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 48 - protein, 217 aa, Artificial Sequence
  Description of Artificial Sequence Synthetic polypeptide
APEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 49 - protein, 217 aa, Artificial Sequence
  Description of Artificial Sequence Synthetic polypeptide
APEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 50 - protein, 217 aa, Artificial Sequence
  Description of Artificial Sequence Synthetic polypeptide
APELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 51 - protein, 10 aa, Artificial Sequence
  Description of Artificial Sequence Synthetic peptide
GFNIKDYYMN

SEQ ID NO: 52 - protein, 17 aa, Artificial Sequence
  Description of Artificial Sequence Synthetic peptide
WIDPDNGNTIYDPKFQG

SEQ ID NO: 53 - protein, 6 aa, Artificial Sequence
  Description of Artificial Sequence Synthetic peptide
RLLMDY

SEQ ID NO: 54 - protein, 16 aa, Artificial Sequence
  Description of Artificial Sequence Synthetic peptide
RSTKSLLHFNGNTYLF

SEQ ID NO: 55 - protein, 7 aa, Artificial Sequence
  Description of Artificial Sequence Synthetic peptide
YMSNLAS

SEQ ID NO: 56 - protein, 9 aa, Artificial Sequence
  Description of Artificial Sequence Synthetic peptide
MQSLEYPFT

SEQ ID NO: 57 - protein, 115 aa, Artificial Sequence
  Description of Artificial Sequence Synthetic polypeptide
EVQLQQSGAELVRPGAFVKLSCKASGFNIKDYYMNWVLQRPEQGLEWIGWIDPDNGNTIYDPKFQGKASITADTSSNTAYLQLSSLTSEDTAVYYCARRLLMDYWGQGTSVTVSS

SEQ ID NO: 58 - protein, 112 aa, Artificial Sequence
  Description of Artificial Sequence Synthetic polypeptide
DIVMTQAAPSVPVTPGESVSISCRSTKSLLHFNGNTYLFWFLQRPGQSPQRLIYYMSNLASGVPDRFSGRGSGTDFTLRISRVEAEDVGVYYCMQSLEYPFTFGTGTKLEIK